(19) United States
(12) Patent Application Publication
(10) Pub. No.: US 2025/0011468 A1
(43) Pub. Date: Jan. 9, 2025

(54) Procoagulant Antibodies

(71) Applicant: Novo Nordisk A/S, Bagsvaerd (DK)

(72) Inventors: Karina Thorn, Farum (DK); Bjarne Gram Hansen, Alleroed (DK); Laust Bruun Johnsen, Skodsborg (DK); Mikkel Nors Harndahl, Roskilde (DK); Zhiru Yang, Beijing (CN); Henrik Oestergaard, Oelstykke (DK); Per J. Greisen, Sammamish, WA (US); Eva Johansson, Bagsvaerd (DK); Morten Groenbech Rasch, Smoerum (DK); Jianhe Chen, Beijing (CN); Anders Svensson, Malmoe (SE); Haisun Zhu, Norfolk, MA (US); Rong Zhou, Beijing (CN); Prafull S. Gandhi, Ballerup (DK)

(21) Appl. No.: 18/782,576

(22) Filed: Jul. 24, 2024

Related U.S. Application Data
(63) Continuation of application No. 16/481,108, filed on Jul. 26, 2019, now Pat. No. 12,084,512.

(30) Foreign Application Priority Data
Feb. 1, 2017 (WO) PCT/CN2017/072796
Oct. 10, 2017 (WO) PCT/CN2017/105556
Dec. 8, 2017 (WO) PCT/CN2017/115210
Jan. 31, 2018 (EP) 18154489.1

(51) Int. Cl. C07K 16/36; A61K 39/00; A61P 7/04

(52) U.S. Cl. CPC C07K 16/36; A61K 39/001154; A61P 7/04; A61K 2039/505; C07K 2317/31; C07K 2317/34; C07K 2317/55; C07K 2317/75; C07K 2317/92

(57) ABSTRACT

The present invention relates to improved procoagulant antibodies including bispecific antibodies capable of binding to coagulation Factor IX (FIX) or the activated form thereof Factor IXa (FIXa) and optionally Factor X (FX) and the activated form thereof Factor Xa (FXa) and promoting FX activation by FIXa, antibodies binding their epitopes and methods and composition for treating subjects suffering from a coagulopathy such as haemophilia A.

### CROSS-REFERENCE TO RELATED APPLICATIONS

This application is a continuation of U.S. application Ser. No. 16/481,108, filed Jul. 26, 2019, which is a 35 U.S.C. § 371 National Stage application of International Application PCT/EP2018/052550 (WO 2018/141863), filed Feb. 1, 2018, which claims priority to International Applications PCT/CN2017/072796, filed Feb. 1, 2017, PCT/CN2017/105556, filed Oct. 10, 2017, PCT/CN2017/115210, filed Dec. 8, 2017 and European Patent Application 18154489.1, filed Jan. 31, 2018; the contents of which are incorporated herein by reference.

### INCORPORATION BY REFERENCE OF THE SEQUENCE LISTING

The instant application contains a Sequence Listing which has been submitted in ASCII format via EFS-Web and is hereby incorporated by reference in its entirety. Said ASCII copy, created on Jun. 28, 2024, is named “160163US03”, and is 197 kilobytes in size.

### BACKGROUND

In patients with a coagulopathy, such as in human beings with haemophilia A and B, various steps of the coagulation cascade are rendered dysfunctional due to, for example, the absence or insufficient presence of a functional coagulation factor. Such dysfunction of one part of the coagulation cascade results in insufficient blood coagulation and potentially life-threatening bleeding, or damage to internal organs, such as the joints.

Coagulation Factor VIII (FVIII) deficiency, commonly referred to as haemophilia A, is a congenital bleeding disorder affecting approximately 420,000 people worldwide, of which around 105,000 are currently diagnosed.

Patients with haemophilia A may receive coagulation factor replacement therapy such as exogenous FVIII. Conventional treatment consists of replacement therapy, provided as prophylaxis or on demand treatment of bleeding episodes. Until recently prophylactic treatment for a patient with severe haemophilia A was up to three intravenous injections/week with either plasma derived FVIII or recombinant FVIII or long-acting variants thereof.

However, such patients are at risk of developing neutralizing antibodies, so-called inhibitors, to such exogenous factors, rendering formerly efficient therapy ineffective. Haemophilia A patients with inhibitors is a non-limiting example of a coagulopathy that is partly congenital and partly acquired. Patients that have developed inhibitors to FVIII cannot be treated with conventional replacement therapy. Recently, a new drug, Hemlibra, has been approved for subcutaneous prophylactic treatment of Haemophilia A with inhibitors. Exogenous coagulation factors may only be administered intravenously, which is of considerable inconvenience and discomfort to patients. For example, infants and toddlers may have to have intravenous catheters surgically inserted into a chest vein, in order for venous access to be guaranteed. This leaves them at great risk of developing bacterial infections. Thus, even with the entry of Hemlibra there is a need for alternative subcutaneous treatment in haemophilia with inhibitors.

In a bleeding individual, coagulation is initiated by formation of the Tissue Factor/Factor VIIa (TF/FVIIa) complex when extravascular TF is exposed to activated FVII (FVIIa) in the blood. TF/FVIIa complex formation leads to the activation of coagulation Factor X (FX) to activated coagulation Factor Xa (FXa) which, together with activated coagulation Factor V (FVa), generates a limited amount of thrombin, which in turn activates blood platelets. Activated platelets support the assembly of the tenase complex composed of activated Factor VIII (FVIIIa) and activated coagulation Factor IX (FIXa). The tenase complex is a very efficient catalyst of FX activation and FXa generated in this second step serves as the active protease in the FVa/FXa pro-thrombinase complex which is responsible for the final thrombin burst. Thrombin cleaves fibrinogen to generate fibrin monomers, which polymerise to form a fibrin network which seals the leaking vessel and stops the bleeding. The rapid and extensive thrombin burst is a prerequisite for the formation of a solid and stable fibrin clot.

An inadequate FXa formation and decreased thrombin generation caused by reduced or absent FVIII activity is the reason underlying the bleeding diathesis in haemophilia A patients.

As mentioned, proteolytic conversion of FX into its enzymatically active form FXa can be achieved by the intrinsic FX-activating complex comprising FIXa and its cofactor FVIIIa. Cofactor binding increases the enzymatic activity of FIXa by about five orders of magnitude and is believed to result through multiple mechanisms as outlined by Scheiflinger et al. (2008) J Thromb Haemost, 6:315-322. Notably, FVIIIa has been found to stabilize a conformation of FIXa that has increased proteolytic activity towards FX (Kolkman J A, Mertens K (2000) Biochemistry, 39:7398-7405, Zögg T, Brandstetter H (2009) Biol Chem, 390:391-400). Based on this observation and realizing that antibodies are versatile binding proteins capable of mimicking a variety of protein-protein interactions, Scheiflinger et al. performed a screen for agonistic anti-FIXa antibodies characterized by an ability to enhance FX activation by FIXa in the presence of a phospholipid surface and calcium, but in the absence of the natural cofactor FVIIIa. From a screen of 5280 hybridoma supernatants, 88 were found to produce antibodies exhibiting various degrees of FIXa agonistic activity, cf. EP1220923 B1 and EP1660536 B1. With respect to the kinetics of FX activation and ability to stimulate thrombin generation in FVIII-deficient human plasma, EP1660536 B1 consistently points to 224F3 as the most efficient antibody (cf. e.g. sections 0060 and 0062). ACE910 or Emicizumab (trade name Hemlibra®) is a humanized, bispecific anti-FIX(a)/anti-FX(a) monoclonal antibody developed by Chugai Pharmaceutical for the treatment of haemophilia A. ACE910 is designed to mimic FVIII cofactor function (see Sampei et al.: (2013) PLoS One, 8, e57479 and WO2012067176).

There are still many unmet medical needs in the haemophilia community, in particular, and in subjects with coagulopathies, in general and the present invention relates to improved compounds capable of substituting for FVIII and thus being useful for the treatment of a coagulopathy such as haemophilia A.

### SUMMARY

The present invention relates to compounds, which serve as a substitute for coagulation Factor VIII (FVIII) in patients suffering from a coagulopathy and in particular patients lacking functional FVIII, such as haemophilia A patients including haemophilia A patients with inhibitors.

Hence, one aspect of the present invention relates to compounds capable of enhancing the generation of FXa and thus partially or completely restore coagulation in patients lacking FVIII.

In one aspect, the compound is an antibody. In one such aspect, the compound is a multispecific antibody such as a bispecific antibody.

In one particular aspect, the invention relates to procoagulant antibodies which serve as a substitute for FVIII in patients lacking FVIII, such as haemophilia A patients.

In one such aspect, the antibody binds to and increases the enzymatic activity of FIXa towards FX, optionally also binding FX.

In one aspect, the invention relates to a procoagulant antibody that binds FX, including bispecific procoagulant antibodies which increase the enzymatic activity of FIXa towards FX and binding FX.

In one aspect, the invention relates to a procoagulant bispecific antibody that is capable of binding to coagulation FIX/FIXa and FX/FXa.

In one aspect, the antibody is human or humanised.

A further aspect of the invention relates to the individual antibodies or antigen-binding fragment thereof that are part of a procoagulant antibody, such as a particular anti-FIX or anti-FIXa antibody or antigen-binding fragment thereof. A further aspect of the invention relates to the individual antibodies or antigen-binding fragment thereof that are part of a procoagulant antibody, such as a particular anti-FX or anti-FXa antibody or antigen-binding fragment thereof.

A further aspect of the invention relates to the manufacture of the antibodies—and intermediates thereof—as disclosed herein.

A further aspect of the invention relates to an antibody that competes with a procoagulant antibody or antigen-binding fragment hereof, as disclosed herein, for binding to FIX/FIXa.

A further aspect of the invention relates to a procoagulant antibody that competes with an antibody or antigen-binding fragment hereof, as disclosed herein, for binding to FX/FXa.

A still further aspect of the invention relates to a pharmaceutical composition comprising a procoagulant antibody as disclosed herein formulated for the delivery of said antibody for the prevention and/or treatment of a coagulopathy.

A further aspect of the invention is directed to the procoagulant antibodies disclosed herein for prevention and/or treatment of a coagulopathy, a disease accompanying coagulopathy, or a disease caused by coagulopathy.

The invention may also solve further problems that will be apparent from the disclosure of the exemplary embodiments.

### BRIEF DESCRIPTION OF THE FIGURES

FIGS. 1A-1J show aligned sequences of SEQ ID NOs:3-188 wherein Complementarity Determining Region 1, 2 and 3 (CDR1, CDR2 and CDR3) of the heavy chain and light chain variable domains have been highlighted consecutively in boxes. FIG. 1A shows sequence alignment of anti-FIX VH domain sequences of 0-1998 lineage. FIG. 1B shows sequence alignment of anti-FIX VL domain sequences of 0-1998 lineage. FIG. 1C shows sequence alignment of anti-FIX VH domain sequences of 0-1886 lineage. FIG. 1D shows sequence alignment of anti-FIX VL domain sequences of 0-1886 lineage. FIG. 1E shows sequence alignment of anti-FIX VH domain sequences of 1-1307 lineage. FIG. 1F shows sequence alignment of anti-FIX VL domain sequences of 1-1307 lineage. FIG. 1G shows sequence alignment of anti-FIX VH domain sequences of other lineage. FIG. 1H shows sequence alignment of anti-FIX VL domain sequences of other lineage. FIG. 1I shows sequence alignment of anti-FX VH domain sequences. FIG. 1J shows sequence alignment of anti-FX VL domain sequences.

FIGS. 2A-2E show Thrombin generation test (TGT) data from the bispecific antibodies mAb4-7761 (FIG. 2A), mAb4-7762 (FIG. 2B), mAb4-7789 (FIG. 2C), mAb5-0057 (FIG. 2D), mAb5-1409 (FIG. 2E) as compared with ACE910 in human tissue factor activated haemophilia A platelet-poor plasma (HA-PPP). The experiment was performed as described in Example 17. Dotted and stippled lines indicate the peak thrombin level (nM) observed in the absence of anti-FVIII antibody in HA-PPP and normal PPP, respectively, and with their standard deviation indicated by dotted lines. The profiles of mAb4-7761 (FIG. 2A), mAb4-7762 (FIG. 2A), mAb4-7789 (FIG. 2C), mAb5-0057 (FIG. 2D), and mAb5-1409 (FIG. 2E) are indicated by up-pointing triangles, whereas that of ACE910 is indicated by down-pointing triangles. FIGS. 2A-2E refer to independent experiments; within each of these experiments the peak thrombin level at each antibody concentration represents the mean±standard deviation of at least three independent runs.

FIG. 3A and FIG. 3B show Thrombin generation test (TGT) data from the bispecific antibodies mAb5-0057 and ACE910 (FIG. 3A), mAb5-1409 and ACE910 (FIG. 3B) in human tissue factor activated haemophilia A platelet-rich plasma (HA-PRP). The experiment was performed as described in Example 17. Dotted and stippled lines indicate the peak thrombin level (nM) observed in the absence of anti-FVIII antibody in HA-PRP and normal PRP, respectively, and with their standard deviation indicated by the dotted lines. The profiles of mAb5-0057 (FIG. 3A) and mAb5-1409 (FIG. 3B) are indicated by up-pointing triangles, whereas that of ACE910 is indicated by down-pointing triangles. Results are shown as mean±standard deviation from four independent experiments.

FIG. 4 shows Thrombin generation test (TGT) data from the monovalent one-armed (OA) antibodies of mAb1-9016 and 224F3 in human Factor XIa activated platelet-poor plasma. The experiment was performed as described in Example 18. Stippled lines indicate the average peak thrombin level (nM) observed in the absence of antibody and with its standard deviation (±1 SD) indicated by dotted lines. The profiles of OA versions of mAb1-9016 and 224F3 (mAb1-1582) are indicated by up-pointing and down-pointing triangles, respectively.

### BRIEF DESCRIPTION OF THE SEQUENCES

SEQ ID NO:1 is the amino acid sequence of human coagulation Factor IX.

SEQ ID NO:2 is the amino acid sequence of human coagulation Factor X.

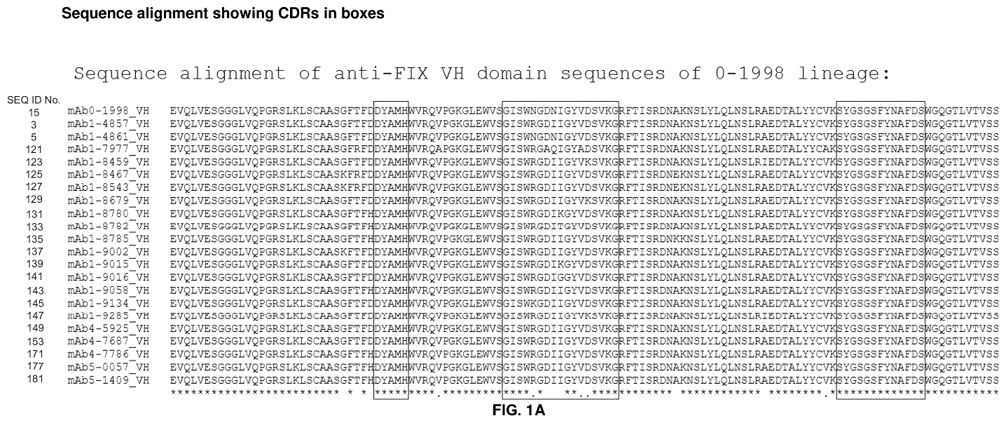

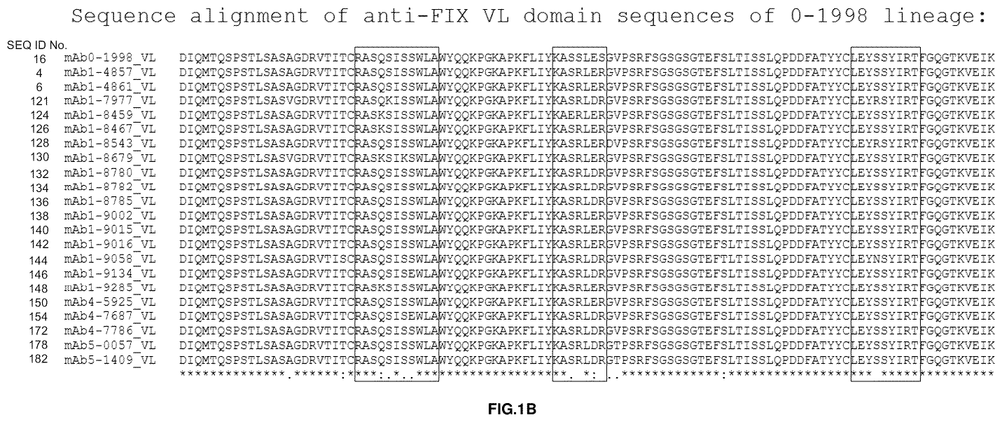

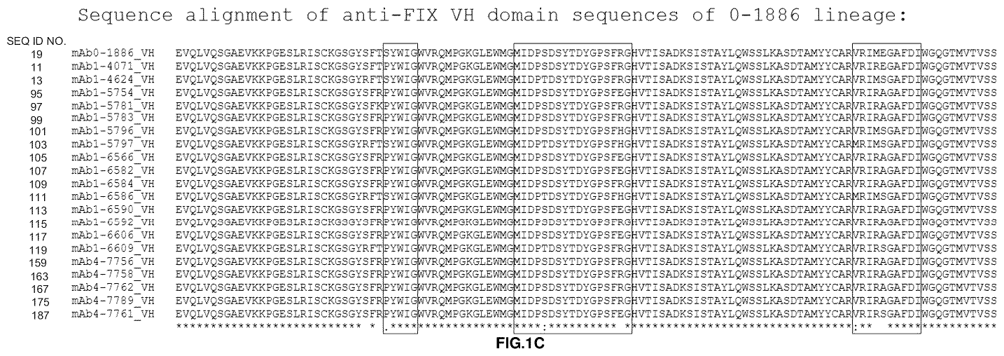

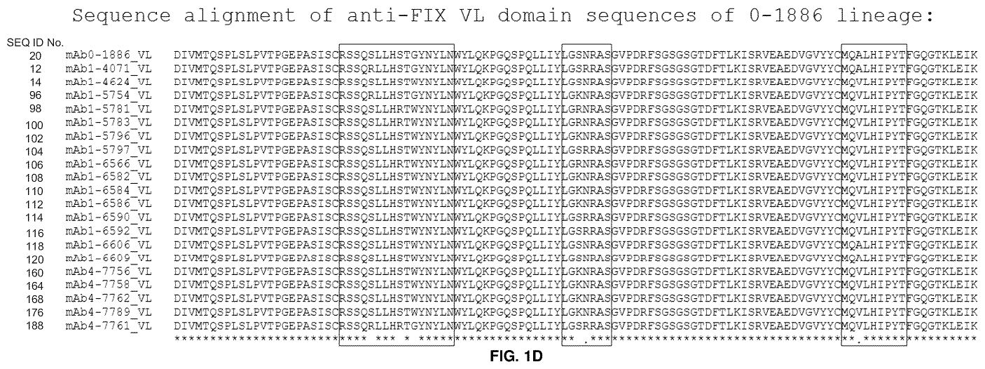

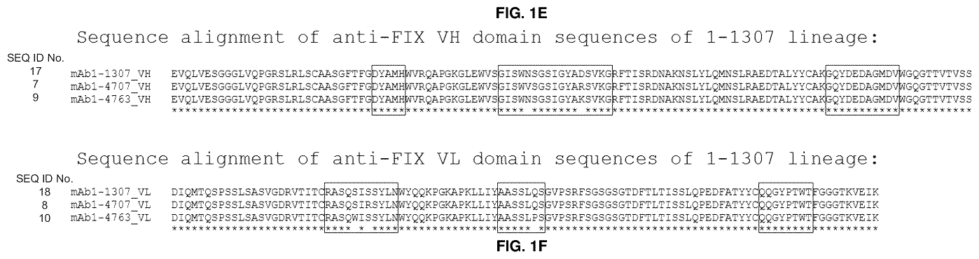

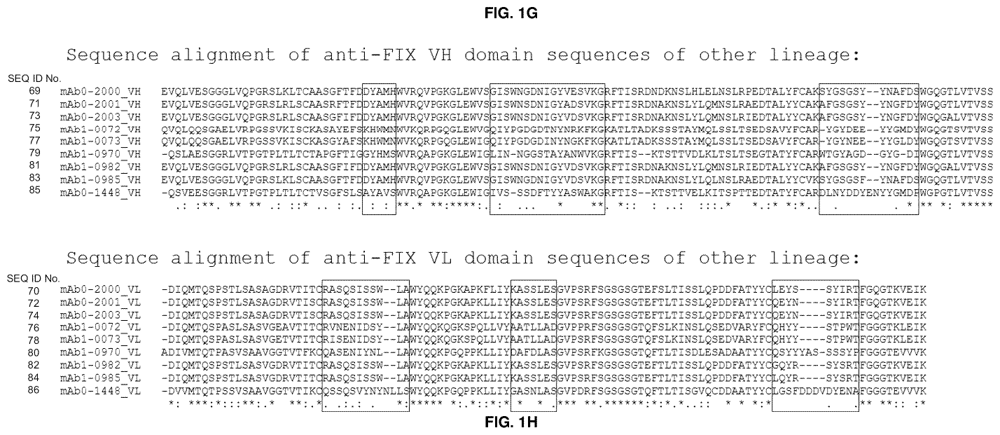

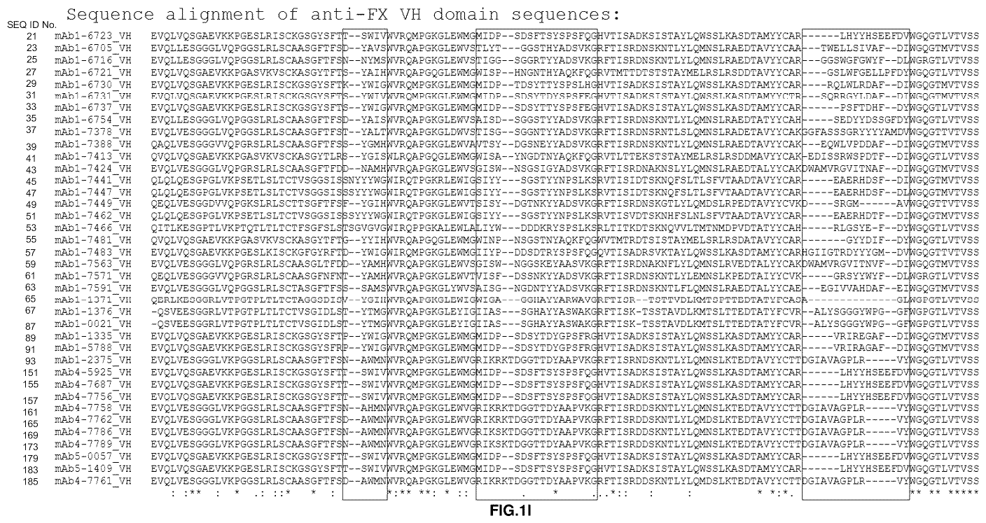

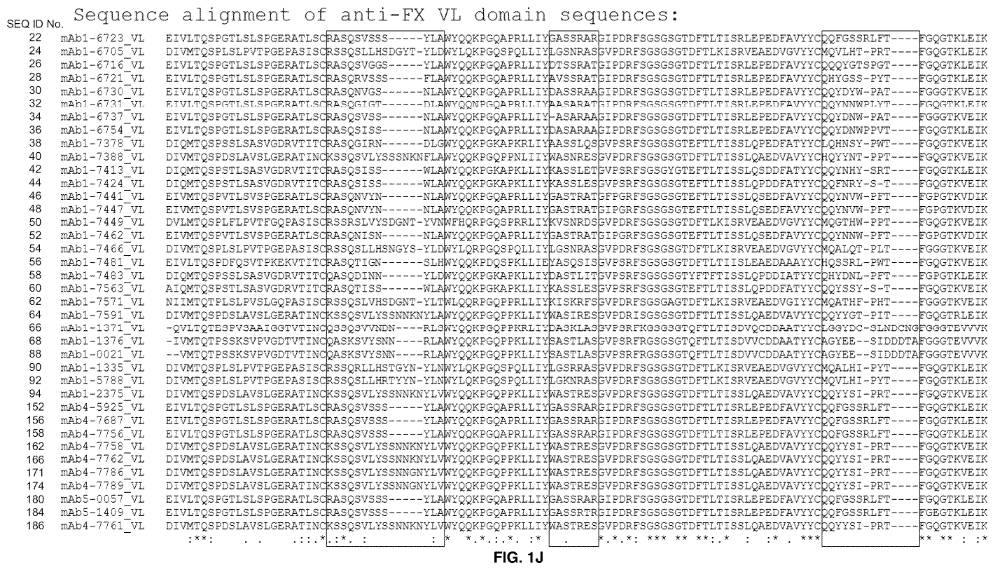

SEQ ID NO:3-188 are the sequences of the heavy chain variable domains (V_{H}) and light chain variable domains (V_{L}) of anti-FIX and anti-FX monoclonal antibodies (mAbs) described herein. IDs for corresponding one-armed (OA) antibodies as well as certain bispecific antibodies are also shown in the table. CDR1-3 sequences are highlighted in boxes in FIG. 1.

Overview of antibody abbreviations, target and SEQ ID NOs for corresponding V_{H} and V_{L} sequences:

| OA or bispecific | | | SEQ ID NO | SEQ ID NO |
|---|---|---|---|---|
| antibody ID | mAb ID | Target | (V_{H}) | (V_{L}) |
| | 1-4857 | FIX | 3 | 4 |
| | 1-4861 | FIX | 5 | 6 |
| | 1-4707 | FIX | 7 | 8 |
| | 1-4763 | FIX | 9 | 10 |
| | 1-4071 | FIX | 11 | 12 |
| | 1-4624 | FIX | 13 | 14 |
| 4-0004 | 0-1998 | FIX | 15 | 16 |
| 3-3279 | 1-1307 | FIX | 17 | 18 |
| 4-0673 | 0-1886 | FIX | 19 | 20 |
| 4-6934 | 1-6723 | FX | 21 | 22 |
| | 1-6705 | FX | 23 | 24 |
| | 1-6716 | FX | 25 | 26 |
| | 1-6721 | FX | 27 | 28 |
| | 1-6730 | FX | 29 | 30 |
| | 1-6731 | FX | 31 | 32 |
| | 1-6737 | FX | 33 | 34 |
| | 1-6754 | FX | 35 | 36 |
| | 1-7378 | FX | 37 | 38 |
| | 1-7388 | FX | 39 | 40 |
| | 1-7413 | FX | 41 | 42 |
| | 1-7424 | FX | 43 | 44 |
| | 1-7441 | FX | 45 | 46 |
| | 1-7447 | FX | 47 | 48 |
| | 1-7449 | FX | 49 | 50 |
| | 1-7462 | FX | 51 | 52 |
| | 1-7466 | FX | 53 | 54 |
| | 1-7481 | FX | 55 | 56 |
| | 1-7483 | FX | 57 | 58 |
| | 1-7563 | FX | 59 | 60 |
| | 1-7571 | FX | 61 | 62 |
| | 1-7591 | FX | 63 | 64 |
| | 1-1371 | FX | 65 | 66 |
| | 1-1376 | FX | 67 | 68 |
| | 0-2000 | FIX | 69 | 70 |
| | 0-2001 | FIX | 71 | 72 |
| | 0-2003 | FIX | 73 | 74 |
| | 1-0072 | FIX | 75 | 76 |
| | 1-0073 | FIX | 77 | 78 |
| | 1-0970 | FIX | 79 | 80 |
| | 1-0982 | FIX | 81 | 82 |
| | 1-0985 | FIX | 83 | 84 |
| | 0-1448 | FIX | 85 | 86 |
| | 1-0021 | FX | 87 | 88 |
| | 1-1335 | FIX | 89 | 90 |
| | 1-5788 | FIX | 91 | 92 |
| | 1-2375 | FX | 93 | 94 |
| 4-3461 | 1-5754 | FIX | 95 | 96 |
| 4-3486 | 1-5781 | FIX | 97 | 98 |
| 4-3490 | 1-5783 | FIX | 99 | 100 |
| 4-3503 | 1-5796 | FIX | 101 | 102 |
| 4-3505 | 1-5797 | FIX | 103 | 104 |
| 4-5337 | 1-6566 | FIX | 105 | 106 |
| 4-5347 | 1-6582 | FIX | 107 | 108 |
| 4-5355 | 1-6584 | FIX | 109 | 110 |
| 4-5342 | 1-6586 | FIX | 111 | 112 |
| 4-5357 | 1-6590 | FIX | 113 | 114 |
| 4-5344 | 1-6592 | FIX | 115 | 116 |
| 4-5368 | 1-6606 | FIX | 117 | 118 |
| 4-5375 | 1-6609 | FIX | 119 | 120 |
| 4-9578 | 1-7977 | FIX | 121 | 122 |
| 5-0900 | 1-8459 | FIX | 123 | 124 |
| 5-0908 | 1-8467 | FIX | 125 | 126 |
| 5-0514 | 1-8543 | FIX | 127 | 128 |
| 5-0658 | 1-8679 | FIX | 129 | 130 |
| 5-0144 | 1-8780 | FIX | 131 | 132 |
| 5-0152 | 1-8782 | FIX | 133 | 134 |
| 5-0161 | 1-8785 | FIX | 135 | 136 |
| 5-1257 | 1-9002 | FIX | 137 | 138 |
| 5-1263 | 1-9015 | FIX | 139 | 140 |
| 5-1270 | 1-9016 | FIX | 141 | 142 |
| 5-1528 | 1-9058 | FIX | 143 | 144 |
| 5-1695 | 1-9134 | FIX | 145 | 146 |
| 5-2035 | 1-9285 | FIX | 147 | 148 |
| 4-5925 | 1-4857/1-6723 | FIX/FX | 149, 151 | 150, 152 |
| 4-7687 | 1-6037/1-6723 | FIX/FX | 153, 155 | 154, 156 |
| 4-7756 | 1-6584/1-6723 | FIX/FX | 159, 157 | 160, 158 |
| 4-7758 | 1-6584/1-6097 | FIX/FX | 163, 161 | 164, 162 |
| 4-7762 | 1-6584/1-6738 | FIX/FX | 167, 165 | 168, 166 |
| 4-7786 | 1-6081/1-6463 | FIX/FX | 171, 169 | 172, 170 |
| 4-7789 | 1-6584/1-6463 | FIX/FX | 175, 173 | 176, 174 |
| 5-0057 | 1-8768/1-6723 | FIX/FX | 177, 179 | 178, 180 |
| 5-1409 | 1-8768/1-7503 | FIX/FX | 181, 183 | 182, 184 |
| 4-7761 | 1-5743/1-6738 | FIX/FX | 187, 185 | 188, 186 |

The first column (“OA or bispecific antibody ID”) contains abbreviations for monovalent one-armed (OA) antibodies and/or bispecific antibodies. The second column (“mAb ID”) represents abbreviations for corresponding component antibodies (for bispecific antibodies, the first mentioned antibody in the second column is an anti-FIX/FIXa antibody and the second is an anti-FX/FXa antibody). Fourth (“SEQ ID NO (V_{H})”) and fifths (“SEQ ID NO (V_{L})”) columns represent SEQ ID NOs for V_{H} and V_{L} sequences, respectively, with the first SEQ ID NO in each column representing the anti-FIX/FIXa antibody and the second the anti-FX/FXa antibody.

### DESCRIPTION

In subjects with a coagulopathy, such as in human beings with haemophilia A, the coagulation cascade is rendered dysfunctional due to the absence or insufficient presence of functional FVIII. Such dysfunction of one part of the coagulation cascade results in insufficient blood coagulation and potentially life-threatening bleeding, or damage to internal organs, such as the joints. The present invention relates to compounds, which serve as a substitute for coagulation Factor VIII (FVIII) in patients suffering from a coagulopathy and in particular patients lacking functional FVIII, such as haemophilia A patients including haemophilia A patients with inhibitors. In one aspect, such compound is an antibody.

In particular the inventors of the present invention have surprisingly identified antibodies which mimic FVIII cofactor activity with high potency and efficacy.

In one particular aspect, the invention relates to procoagulant antibodies which serve as a substitute for FVIII in patients lacking functional FVIII, such as haemophilia A patients.

In one such aspect, the procoagulant antibodies bind to and increase the enzymatic activity of coagulation Factor IXa (FIXa) towards coagulation Factor X (FX), optionally also binding FX. In one such aspect the antibodies of the invention are bispecific antibodies capable of binding to FIX/FIXa and FX.

### Coagulation Factor IX

FIX is a vitamin K-dependent coagulation factor with structural similarities to Factor VII, prothrombin, Factor X, and Protein C. The circulating zymogen form consists of 415 amino acids divided into four distinct domains comprising an N-terminal γ-carboxyglutamic acid-rich (Gla) domain, two EGF domains and a C-terminal trypsin-like serine protease domain. FIX circulates in plasma as a single-chain zymogen (SEQ ID NO:1). Activation of FIX occurs by limited proteolysis at Arg145 and Arg180 to release the activation peptide (residues 146 to 180 of SEQ ID NO:1). Thus, activated FIX (FIXa) is composed of residues 1-145 of SEQ ID NO:1 (light chain) and residues 181-415 of SEQ ID NO:1 (heavy chain).

Circulating FIX molecules thus comprise the FIX zymogen and the activated form of FIX which are herein generally referred to as FIX and FIXa with reference to SEQ ID NO:1. Activated Factor IX is referred to as Factor IXa or FIXa. The term “FIX (SEQ ID NO:1) and/or the activated form thereof (FIXa)” may also be referred to as “FIX/FIXa” or “FIX(a)”.

FIXa is a trypsin-like serine protease that serves a key role in haemostasis by generating, as part of the tenase complex, most of the Factor Xa required to support proper thrombin formation during coagulation.

FIX is herein represented by SEQ ID NO:1 corresponding to the Ala148 allelic form of human FIX (Anson et al. EMBO J. 1984 3:1053-1060; McGraw et al., Proc Natl Acad Sci USA. 1985 82:2847-2851; Graham et al. Am. J. Hum. Genet. 1988 42:573-580). In the present invention FIX is intended to cover all natural variants of FIX, such as the T148 variant (Uniprot ID P00740).

### Coagulation Factor X

FX is a vitamin K-dependent coagulation factor with structural similarities to Factor VII, prothrombin, FIX, and protein C. Human FX zymogen comprises four distinct domains comprising an N-terminal gamma-carboxyglutamic acid rich (Gla) domain, two EGF domains, and a C-terminal trypsin-like serine protease domain. FX circulates in plasma as a two-chain zymogen including residues 1-139 of SEQ ID NO:2 (light chain) and residues 143-448 of SEQ ID NO:2 (heavy chain). Activation of FX occurs by limited proteolysis at Arg194, which results in the release of the activation peptide (Aa143-194). Thus, activated FX (FXa) is composed of residues 1-139 of SEQ ID NO:2 (light chain) and residues 195-448 of SEQ ID NO:2 (activated heavy chain). Circulating FX molecules thus comprises the FX zymogen and the activated form of FX which are herein referred to as FX and FXa, respectively, with reference to SEQ ID NO:2. In the present invention FX is intended to cover all natural variants of FX. The term “FX (SEQ ID NO:2) and/or the activated form thereof (FXa)” may also be referred to as “FX/FXa” or “FX(a)”.

### Antibodies

The term “antibody” herein refers to a protein, derived from an immunoglobulin sequence, which is capable of binding to an antigen or a portion thereof. The term antibody includes, but is not limited to, full length antibodies of any class (or isotype), that is, IgA, IgD, IgE, IgG, IgM and/or IgY. The term antibody includes—but is not limited to—antibodies that are bivalent, such as bispecific antibodies.

Natural full-length antibodies comprise at least four polypeptide chains: two heavy chains (HC) and two light chains (LC) that are connected by disulfide bonds. In some cases, natural antibodies comprise less than four chains, as in the case of the IgNARs found in Chondrichthyes. One class of immunoglobulins of particular pharmaceutical interest is the IgGs. In humans, the IgG class may be divided into four sub-classes IgG1, IgG2, IgG3 and IgG4, based on the sequence of their heavy chain constant regions. The light chains can be divided into two types, kappa and lambda chains, based on differences in their sequence composition. IgG molecules are composed of two heavy chains, interlinked by two or more disulfide bonds, and two light chains, each attached to a heavy chain by a disulfide bond. An IgG heavy chain may comprise a heavy chain variable domain (V_{H}) and up to three heavy chain constant (C_{H}) domains: C_{H}1, C_{H}2 and C_{H}3. A light chain may comprise a light chain variable domain (V_{L}) and a light chain constant domain (C_{L}). V_{H} and V_{L} regions can be further subdivided into regions of hypervariability, termed complementarity determining regions (CDRs) or hypervariable regions (HvRs), interspersed with regions that are more conserved, termed framework regions (FR). V_{H} and V_{L} domains are typically composed of three CDRs and four FRs, arranged from amino-terminus to carboxy-terminus in the following order: FR1, CDR1, FR2, CDR2, FR3, CDR3, FR4. The heavy and light chain variable domains containing the hypervariable regions (CDRs) form a structure that is capable of interacting with an antigen, whilst the constant region of an antibody may mediate binding of the immunoglobulin to host tissues or factors, including, but not limited to various cells of the immune system (effector cells), Fc receptors and the first component, C1q, of the C1 complex of the classical complement system.

Antibodies of the invention may be monoclonal antibodies (mAbs), in the sense that they represent a set of unique heavy and light chain variable domain sequences as expressed from a single B-cell or by a clonal population of B cells. Antibodies of the invention may be produced and purified using various methods that are known to the person skilled in the art. For example, antibodies may be produced from hybridoma cells. Antibodies may be produced by B-cell expansion. Antibodies or fragment thereof may be recombinantly expressed in mammalian or microbial expression systems, or by in vitro translation.

Antibodies or fragment thereof may also be recombinantly expressed as cell surface bound molecules, by means of e.g. phage display, bacterial display, yeast display, mammalian cell display or ribosome or mRNA display.

Antibodies of the current invention may be isolated. The term “isolated antibody” refers to an antibody that has been separated and/or recovered from (an)other component(s) in the environment in which it was produced and/or that has been purified from a mixture of components present in the environment in which it was produced.

Certain antigen-binding fragments of antibodies may be suitable in the context of the current invention, as it has been shown that the antigen-binding function of an antibody can be performed by fragments of a full-length antibody. The term “antigen-binding fragment” of an antibody refers to one or more fragment(s) of an antibody that retain(s) the ability to specifically bind to or recognise an antigen, such as FIX/FIXa, FX/FXa or another target molecule, as described herein. Examples of antigen-binding fragments include (but is not limited to) Fab, Fab′, Fab₂, Fab′₂, Fv (typically the combination of V_{L} and V_{H} domains of a single arm of an antibody), single-chain Fv (scFv); see e.g. Bird et al. Science 1988; 242:423-426; and Huston et al. PNAS 1988; 85:5879-5883), dsFv, Fd (typically the V_{H} and C_{H}1 domain), monovalent molecules comprising both a single V_{H} and a single V_{L} domain; minibodies, diabodies, triabodies, tetrabodies, and kappa bodies (see, e.g. Ill et al (1997) Protein Eng 10: 949-57); as well as one or more isolated CDRs or a functional paratope, where the isolated CDRs or antigen-binding residues or polypeptides can be associated or linked together so as to form a functional antibody fragment. These antibody fragments may be obtained using conventional techniques known to those skilled in the art, and the fragments may be screened for utility in the same manner as intact antibodies.

“Fab fragments” of an antibody, including “Fab” and “Fab′₂” fragments, can be derived from an antibody by cleavage of the heavy chain in the hinge region on the N-terminal or C-terminal side, respectively, of the hinge cysteine residues connecting the heavy chains of the antibody. A “Fab” fragment includes the variable and constant domains of the light chain and the variable domain and C_{H}1 domain of the heavy chain. “Fab′₂” fragments comprise a pair of “Fab′” fragments that are generally covalently linked by their hinge cysteines. A Fab′ is formally derived from a Fab′₂ fragment by cleavage of the hinge disulfide bonds connecting the heavy chains in the Fab′₂. Other chemical couplings than disulfide linkages of antibody fragments are also known in the art. A Fab fragment retains the ability of the parent antibody to bind to its antigen, potentially with a lower affinity. Fab′₂ fragments are capable of bivalent binding, whereas Fab and Fab′ fragments can only bind monovalently. Generally, Fab fragments lack the constant C_{H}2 and C_{H}3 domains, i.e. the Fc part, where interaction with the Fc receptors and C1q would occur. Thus, Fab fragments are in general devoid of effector functions. Fab fragments may be produced by methods known in the art, either by enzymatic cleavage of an antibody, e.g. using papain to obtain the Fab or pepsin to obtain the Fab′₂, Fab fragments including Fab, Fab′, Fab′₂ may be produced recombinantly using techniques that are well known to the person skilled in the art.

An “Fv” (fragment variable) fragment is an antibody fragment that contains a complete antigen recognition and binding site, and generally comprises one heavy and one light chain variable domain in association that can be covalent in nature, for example in a single chain variable domain fragment (scFv). It is in this configuration that the three hypervariable regions of each variable domain interact to define an antigen-binding site on the surface of the V_{H}-V_{L} dimer. Collectively, the six hypervariable regions or a subset thereof confer antigen binding specificity to the antibody.

“Single-chain Fv” or “scFv” antibody comprise the V_{H} and V_{L} domains of antibody, where these domains are present in a single polypeptide chain. Generally, the Fv polypeptide further comprises a polypeptide linker between the V_{H} and V_{L} domains that enables the scFv to form the desired structure for antigen binding. For a review of scFv, see Pluckthun, 1994, In: The Pharmacology of Monoclonal Antibodies, Vol. 113, Rosenburg and Moore eds. Springer-Verlag, New York, pp. 269-315.

“Single-chain Fab” or “scFab” antibody comprise the V_{H}, C_{H}1, V_{L} and C_{L} domains of an antibody, where these domains are present in a single polypeptide chain. Generally, the Fab polypeptide further comprises a polypeptide linker between either V_{H} and C_{L} or V_{L} and C_{H}1 domains that enables the scFab to form the desired structure for antigen binding (Koerber et al. (2015) J Mol Biol. 427:576-86).

The term “diabodies” refers to small antibody fragments with two antigen-binding sites, in which fragments comprise a heavy chain variable domain (V_{H}) connected to a light chain variable domain (V_{L}) in the same polypeptide chain (V_{H} and V_{L}). By using a linker that is too short to allow pairing between the two variable domains on the same chain, the variable domains are forced to pair with complementary domains of another chain, creating two antigen-binding sites.

The expression “linear antibodies” refers to antibodies as described in Zapata et al. (1995) Protein Eng. 8: 1057-1062. Briefly, these antibodies contain a pair of tandem Fd segments (V_{H}-C_{H}1-V_{H}-C_{H}1) that, together with complementary light chain polypeptides, form a pair of antigen binding regions. Linear antibodies can be bispecific or monospecific.

Antibody fragments may be obtained using conventional recombinant or protein engineering techniques and the fragments can be screened for binding to FIX and the activated form thereof, FX or another function, in the same manner as intact antibodies.

Antibody fragments of the invention may be made by truncation, e.g. by removal of one or more amino acids from the N and/or C-terminal ends of a polypeptide. Fragments may also be generated by one or more internal deletions.

An antibody of the invention may be, or may comprise, a fragment of the antibody, or a variant of any one of the antibodies disclosed herein. An antibody of the invention may be, or may comprise, an antigen binding portion of one of these antibodies, or variants thereof. For example, an antibody of the invention may be a Fab fragment of one of these antibodies or variants thereof, or it may be a single chain antibody derived from one of these antibodies, or a variant thereof. Also, an antibody of the invention may be a combination of a full length antibody and fragment thereof.

The term “one-armed” as used herein, refers to a particular type of monovalent antibody constituted by an antibody heavy chain, a truncated heavy chain lacking the Fab region, and a single light chain.

The term “monospecific” antibody as used herein, refers to an antibody which is capable of binding to one particular epitope (including but not limited to bivalent antibodies).

The term “bispecific” antibody as used herein, refers to an antibody which is capable of binding to two different antigens or two different epitopes on the same antigen.

The term “trispecific” antibody as used herein, refers to an antibody which is capable of binding to three different antigens or three different epitopes on the same antigen or three different epitopes present on two different antigens.

The term “multispecific” antibody as used herein, refers to an antibody which is capable of binding to two or more different antigens or two or more different epitopes on the same antigen. Multispecific antibodies thus comprise bi- and trispecific antibodies.

Bispecific antibodies in full length IgG format can be generated by fusion of two individual hybridomas to form a hybrid quadroma which produces a mixture of antibodies including a fraction of bispecific heterodimerising antibodies (Chelius D. et al.; MAbs. 2010 May-June; 2(3): 309-319). Bispecific heterodimerising antibodies may alternatively be produced by using recombinant technologies. Heterodimerisation can also be achieved by engineering the dimerisation interface of the Fc region to promote heterodimerisation. One example hereof is the so-called knob-in-hole mutations where sterically bulky side chains (knobs) are introduced in one Fc matched by sterically small side chains (holes) on the opposite Fc thereby creating steric complementarity promoting heterodimerisation. Other methods for engineered heterodimerisation Fc interfaces are electrostatic complementarity, fusion to non-IgG heterodimerisation domains or utilising the natural Fab-arm exchange phenomenon of human IgG4 to control heterodimerisation. Examples of heterodimerised bispecific antibodies are well described in the literature, e.g. (Klein C, et al.; MAbs. 2012 November-December; 4(6): 653-663).

Special attention has to be paid to the light chains in heterodimeric antibodies. Correct pairing of LCs and HCs can be accomplished by the use of a common light chain. Again engineering of the LC/HC interface can be used to promote heterodimerisation or light chain cross-over engineering as in CrossMabs. In vitro re-assembly under mildly reducing conditions of antibodies from two individual IgGs containing appropriate mutations can also be used to generate bispecific antibodies (e.g. Labrijn et al., PNAS, 110, 5145-5150 (2013)). Also the natural Fab-arm exchange method is reported to ensure correct light chains paring. Multispecific antibody-based molecules may also be expressed recombinantly as fusion proteins combining the natural modules of IgGs to form multispecific and multivalent antibody derivatives as described in the literature. Examples of fusion antibodies are DVD-Igs, IgG-scFV, Diabodies, DARTs etc. Specific detection or purification tags, half-life extension moieties or other components can be incorporated in the fusion proteins. Additional non-IgG modalities may also be incorporated in the fusion proteins. Bispecific full length antibodies based on Fc heterodimerisation are commonly referred to as asymmetric IgGs, irrespective of the LC paring methodology.

Generally, bispecific antibodies may be produced in a variety of molecular formats as reviewed by Brinkmann et al. (Brinkmann et al. The making of bispecific antibodies. Mabs 9, 182-212 (2017)).

Multispecific antibody-based molecules may also be produced by chemical conjugation or coupling of individual full length IgGs or coupling of fragments of IgGs to form multispecific and multivalent antibody derivatives as described in the literature. Examples are chemically coupled Fab fragments, IgG-dimer etc. Specific detection or purification tags, half-life extension molecules or other components can be incorporated in the conjugate proteins. Additional non-IgG polypeptide may also be incorporated in the fusion proteins. Multispecific molecules may also be produced by combining recombinant and chemical methods including those described above.

In one aspect, an antibody of the invention is a chimeric antibody, a human antibody or a humanised antibody. Such antibody can be generated by using, for example, suitable antibody display or immunization platforms or other suitable platforms or methods known in the field. The term “human antibody”, as used herein, is intended to include antibodies having variable domains in which at least a portion of a framework region and/or at least a portion of a CDR region are derived from human germline immunoglobulin sequences. For example, a human antibody may have variable domains in which both the framework and CDR regions are derived from human germline immunoglobulin sequences. Furthermore, if the antibody contains a constant region, the constant region or a portion thereof is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

Such a human antibody may be a human monoclonal antibody. Such a human monoclonal antibody may be produced by a hybridoma which includes a B cell obtained from a transgenic nonhuman animal, e.g., a transgenic mouse, having a genome comprising human immunoglobulin heavy and light chain gene segments repertoires, fused to an immortalised cell.

Human antibodies may be isolated from sequence libraries built on selections of human germline sequences, further diversified with natural and synthetic sequence diversity.

Human antibodies may be prepared by in vitro immunisation of human lymphocytes followed by transformation of the lymphocytes with Epstein-Barr virus.

Human antibodies may be produced by recombinant methods known in the art.

The term “human antibody derivative” refers to any modified form of the human antibody, such as a conjugate of the antibody and another agent or antibody.

The term “humanised antibody”, as used herein, refers to a human/non-human antibody that contains a sequence (CDR regions or parts thereof) derived from a non-human immunoglobulin. A humanised antibody is, thus, a human immunoglobulin (recipient antibody) in which residues from at least a hypervariable region of the recipient are replaced by residues from a hypervariable region of an antibody from a non-human species (donor antibody) such as from a mouse, rat, rabbit or non-human primate, which have the desired specificity, affinity, sequence composition and functionality. In some instances, framework (FR) residues of the human immunoglobulin are replaced by corresponding non-human residues. An example of such a modification is the introduction of one or more so-called back-mutations, which are typically amino acid residues derived from the donor antibody.

Humanisation of an antibody may be carried out using recombinant techniques known to the person skilled in the art (see, e.g., Antibody Engineering, Methods in Molecular Biology, vol. 248, edited by Benny K. Lo). A suitable human recipient framework for both the light and heavy chain variable domain may be identified by, for example, sequence or structural homology. Alternatively, fixed recipient frameworks may be used, e.g., based on knowledge of structure, biophysical and biochemical properties. The recipient frameworks can be germline derived or derived from a mature antibody sequence. CDR regions from the donor antibody can be transferred by CDR grafting. The CDR grafted humanised antibody can be further optimised for e.g. affinity, functionality and biophysical properties by identification of critical framework positions where re-introduction (back-mutation) of the amino acid residue from the donor antibody has beneficial impact on the properties of the humanised antibody. In addition to donor antibody derived back-mutations, the humanised antibody can be engineered by introduction of germline residues in the CDR or framework regions, elimination of immunogenic epitopes, site-directed mutagenesis, affinity maturation, etc. Furthermore, humanised antibodies may comprise residues that are not found in the recipient antibody or in the donor antibody. These modifications are made to further refine antibody performance. In general, a humanised antibody will comprise at least one—typically two—variable domains, in which all or substantially all of the CDR regions correspond to those of a non-human immunoglobulin and in which all or substantially all of the FR residues are those of a human immunoglobulin sequence. The humanised antibody can, optionally, also comprise at least a portion of an immunoglobulin constant region (Fc), typically that of a human immunoglobulin.

The term “humanised antibody derivative” refers to any modified form of the humanised antibody, such as a conjugate of the antibody and a chemical agent or a conjugate of the antibody with another antibody.

The term “chimeric antibody”, as used herein, refers to an antibody comprising portions of antibodies derived from two or more species. For example, the genes encoding such antibody comprise genes encoding variable domains and genes encoding constant domains originated from two different species. For example, the genes encoding variable domains of a mouse monoclonal antibody may be joined to the genes encoding the constant domains of an antibody of human origin.

The fragment crystallisable region (“Fc region”/“Fc domain”) of an antibody is the C-terminal region of an antibody, which comprises the hinge and the constant C_{H}2 and C_{H}3 domains.

The Fc domain may interact with cell surface receptors called Fc receptors, as well as some proteins of the complement system. The Fc region enables antibodies to interact with the immune system. In one aspect of the invention, antibodies may be engineered to include modifications within the Fc region, typically to alter one or more of its functional properties, such as serum half-life, complement fixation, Fc-receptor binding, protein stability and/or antigen-dependent cellular cytotoxicity, or lack thereof, among others. Furthermore, an antibody of the invention may be chemically modified (e.g., one or more chemical moieties can be attached to the antibody) or be modified to alter its glycosylation, again to alter one or more functional properties of the antibody. An IgG1 antibody may carry a modified Fc domain comprising one or more, and perhaps all of the following mutations that will result in decreased affinity to certain Fc-gamma receptors (L234A, L235E, and G237A) and in reduced C1q-mediated complement fixation (A330S and P331 S), respectively (residue numbering according to the EU index). Alternatively, other amino acid substitutions, and combinations thereof and combinations with the above mentioned, known in the art to lead to altered (reduced or increased) Fc-gamma receptor binding may be used.

The isotype of an antibody of the invention may be IgG, such as IgG1, such as IgG2, such as IgG4. If desired, the class of an antibody may be “switched” by known techniques. For example, an antibody that was originally produced as an IgM molecule may be class switched to an IgG antibody. Class switching techniques also may be used to convert one IgG subclass to another, for example: from IgG1 to IgG2 or IgG4; from IgG2 to IgG1 or IgG4; or from IgG4 to IgG1 or IgG2. Engineering of antibodies to generate constant region chimeric molecules, by combination of regions from different IgG subclasses, can also be performed. In one embodiment the hinge region of the antibody is modified such that the number of cysteine residues in the hinge region is altered, e.g., increased or decreased. This approach is described further for instance in U.S. Pat. No. 5,677,425 by Bodmer et al.

The constant region may be modified to stabilise the antibody, e.g., to reduce the risk of a bivalent antibody separating into half antibodies. For example, in an IgG4 constant region, residue S228 (according to the EU numbering index and S241 according to Kabat) may be mutated to a proline (P) residue to stabilise inter heavy chain disulphide bridge formation at the hinge (see, e.g., Angal et al. Mol Immunol. 1993; 30:105-8).

Antibodies or fragment thereof may be defined in terms of their complementarity-determining regions (CDRs). The term “complementarity-determining region” or “hypervariable region”, when used herein, refers to the regions of an antibody in which amino acid residues involved in antigen-binding are situated. The region of hypervariability or CDRs can be identified as the regions with the highest variability in amino acid alignments of antibody variable domains. Databases can be used for CDR identification such as the Kabat database, the CDRs e.g. being defined as comprising amino acid residues 24-34 (L1), 50-56 (L2) and 89-97 (L3) of the light-chain variable domain and 31-35 (H1), 50-65 (H2) and 95-102 (H3) in the heavy-chain variable domain; (Kabat et al. 1991; Sequences of Proteins of Immunological Interest, Fifth Edition, U.S. Department of Health and Human Services, NIH Publication No. 91-3242) Alternatively CDRs can be defined as those residues from a “hypervariable loop” (residues 26-33 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al. supra. Phrases such as “Kabat position”, “Kabat residue”, and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “framework region” or “FR” residues refer to those V_{H} or V_{L} amino acid residues that are not within the CDRs, as defined herein.

An antibody of the invention may comprise a CDR region from one or more of the specific antibodies disclosed herein.

The term “procoagulant antibody” refers to an antibody which potentiates blood coagulation for example by accelerating the process of blood coagulation and/or increasing the enzymatic activity of one or more coagulation factors.

The term “procoagulant activity” refers to the ability of a compound, such as an antibody, to potentiate blood coagulation for example by accelerating the process of blood coagulation and/or increasing the enzymatic activity of one or more coagulation factors.

The term “antigen” (Ag) refers to the molecular entity used for immunisation of an immunocompetent vertebrate to produce the antibody (Ab) that recognizes the Ag. Herein, Ag is termed more broadly and is generally intended to include target molecules that are specifically recognized by the Ab, thus including fragments or mimics of the molecule used in the immunisation process, or other process, e.g. phage display, used for generating the Ab.

The term “epitope”, as used herein, is defined in the context of a molecular interaction between an “antigen binding polypeptide”, such as an antibody (Ab), and its corresponding antigen (Ag). Generally, “epitope” refers to the area or region on an Ag to which an Ab binds, i.e. the area or region in physical contact with the Ab. Physical contact may be defined using various criteria (e.g. a distance cut-off of 2-6 Å, such as 3 Å, such as 3.5 Å such as 4 Å, such as 4.5 Å, such as 5 Å; or solvent accessibility) for atoms in the Ab and Ag molecules. FIX/FIXa and FX/FXa may comprise a number of different epitopes, which may include, without limitation, (1) linear peptide epitopes (2) conformational epitopes which consist of one or more non-contiguous amino acids located near each other in the mature FIX/FIXa or FX/FXa conformation; and (3) epitopes which consist, either in whole or part, of molecular structures covalently attached to FIX/FIXa or FX/FXa, such as carbohydrate groups.

The epitope for a given antibody (Ab)/antigen (Ag) pair can be described and characterized at different levels of detail using a variety of experimental and computational epitope mapping methods. The experimental methods include mutagenesis, X-ray crystallography, Nuclear Magnetic Resonance (NMR) spectroscopy, Hydrogen Deuterium eXchange Mass Spectrometry (HDX-MS) and various competition binding methods; methods that are known in the art. As each method relies on a unique principle, the description of an epitope is intimately linked to the method by which it has been determined. Thus, depending on the epitope mapping method employed, the epitope for a given Ab/Ag pair may be described differently.

In the context of an X-ray derived crystal structure defined by spatial coordinates of a complex between an Ab, e.g. a Fab fragment, and its Ag, the term epitope is herein, unless otherwise specified or contradicted by context, specifically defined as FIX/FIXa or FX residues characterized by having a heavy atom (i.e. a non-hydrogen atom) within a distance of 3.5 Å, from a heavy atom in the Ab.

Epitopes described at the amino acid level, e.g. determined from an X-ray structure, are said to be identical if they contain the same set of amino acid residues. Epitopes are said to overlap if at least one amino acid residue is shared by the epitopes. Epitopes are said to be separate (unique) if no amino acid residue is shared by the epitopes.

The definition of the term “paratope” is derived from the above definition of “epitope” by reversing the perspective. Thus, the term “paratope” refers to the area or region on the Ab to which an Ag binds, i.e. with which it makes physical contact to the Ag.

In the context of an X-ray derived crystal structure, defined by spatial coordinates of a complex between an Ab, such as a Fab fragment, and its Ag, the term paratope is herein, unless otherwise specified or contradicted by context, specifically defined as Ab residues characterized by having a heavy atom (i.e. a non-hydrogen atom) within a distance of 3.5 Å from a heavy atom in FIX/FIXa or FX.

The epitope and paratope for a given antibody (Ab)/antigen (Ag) pair may be identified by routine methods. For example, the general location of an epitope may be determined by assessing the ability of an antibody to bind to different fragments or variants of FIX/FIXa or FX. The specific amino acids within FIX/FIXa or FX that make contact with an antibody (epitope) and the specific amino acids in an antibody that make contact with FIX/FIXa or FX (paratope) may also be determined using routine methods. For example, the antibody and target molecule may be combined and the Ab:Ag complex may be crystallised. The crystal structure of the complex may be determined and used to identify specific sites of interaction between the antibody and its target.

Epitopes on an antigen may comprise one or more hot-spot residues, i.e. residues which are particularly important for the interaction with the cognate antibody, and where interactions mediated by the side chain of said hot-spot residue contribute significantly to the binding energy for the antibody/antigen interaction (Peng et al. (2014) PNAS 111, E2656-E2665). Hot-spot residues can be identified by testing variants of the antigen (here FIX/FIXa and FX), where single epitope residues have been substituted by e.g. alanine, for binding to the cognate antibody. If substitution of an epitope residue with alanine has a strong impact on binding to the antibody, said epitope residue is considered a hot-spot residue, and therefore of particular importance for binding of the antibody to the antigen.

Antibodies that bind to the same antigen can be characterised with respect to their ability to bind to their common antigen simultaneously and may be subjected to “competition binding”/“binning”. In the present context, the term “binning” refers to a method of grouping antibodies that bind to the same antigen. “Binning” of antibodies may be based on competition binding of two antibodies to their common antigen in assays based on standard techniques.

An antibody's “bin” is defined using a reference antibody. If a second antibody is unable to bind to an antigen at the same time as the reference antibody, the second antibody is said to belong to the same “bin” as the reference antibody. In this case, the reference and the second antibody competitively bind the same part of an antigen and are coined “competing antibodies”. If a second antibody is capable of binding to an antigen at the same time as the reference antibody, the second antibody is said to belong to a separate “bin”. In this case, the reference and the second antibody do not competitively bind the same part of an antigen and are coined “non-competing antibodies”.

Antibody “binning” does not provide direct information about the epitope.

Competing antibodies, i.e. antibodies belonging to the same “bin” may have identical epitopes, overlapping epitopes or even separate epitopes. The latter is the case if the reference antibody bound to its epitope on the antigen takes up the space required for the second antibody to contact its epitope on the antigen (“steric hindrance”). Non-competing antibodies generally have separate epitopes. Thus, in some embodiments antibodies of the invention will bind to the same epitope as at least one of the antibodies specifically disclosed herein.

Competition assays for determining whether an antibody competes for binding with, an anti-FIX/FIXa or anti-X antibody disclosed herein are known in the art. Exemplary competition assays include immunoassays (e.g., ELISA assays, RIA assays), surface plasmon resonance analysis (e.g. using a BIAcore™ instrument), biolayer interferometry (ForteBio®) and flow cytometry.

Typically, a competition assay involves the use of an antigen bound to a solid surface or expressed on a cell surface, a test FIX- or FIXa binding antibody and a reference antibody. The reference antibody is labelled and the test antibody is unlabelled. Competitive inhibition is measured by determining the amount of labelled reference antibody bound to the solid surface or cells in the presence of the test antibody. Usually the test antibody is present in excess (e.g., 1, 5, 10, 20, 100, 1000, 10000 or 100000-fold). Antibodies identified as being competitive in the competition assay (i.e., competing antibodies) include antibodies binding to the same epitope, or overlapping epitopes, as the reference antibody, and antibodies binding to an adjacent epitope sufficiently proximal to the epitope bound by the reference antibody for steric hindrance to occur.

In an exemplary competition assay, a reference anti-FIX or anti-FIXa antibody is biotinylated using commercially available reagents. The biotinylated reference antibody is mixed with serial dilutions of the test antibody or unlabelled reference antibody (self-competition control) resulting in a mixture of various molar ratios (e.g., 1, 5, 10, 20, 100, 1000, 10000 or 100000-fold) of test antibody (or unlabelled reference antibody) to labelled reference antibody. The antibody mixture is added to a FIX or FIXa polypeptide coated-ELISA plate. The plate is then washed, and horseradish peroxidase (HRP)-strepavidin is added to the plate as the detection reagent. The amount of labelled reference antibody bound to the target antigen is detected following addition of a chromogenic substrate (e.g., TMB (3,3′,5,5′-tetramethylbenzidine) or ABTS (2,2″-azino-di-(3-ethylbenzthiazoline-6-sulfonate)), which are known in the art. Optical density readings (OD units) are made using a spectrometer (e.g. SpectraMax® M2 spectrometer (Molecular Devices)). The response (OD units) corresponding to zero percent inhibition is determined from wells without any competing antibody. The response (OD units) corresponding to 100% inhibition, i.e. the assay background, is determined from wells without any labelled reference antibody or test antibody. Percent inhibition of labelled reference antibody to FIX or FIXa by the test antibody (or the unlabelled reference antibody) at each concentration is calculated as follows: % inhibition=(1−(OD units−100% inhibition)/(0% inhibition−100% inhibition))*100.

The person skilled in the art will understand that similar assays may be performed to determine if two or more anti-FX/FXa antibodies shares a binding region, a bin and/or competitively binds the antigen. Persons skilled in the art will also appreciate that the competition assay can be performed using various detection systems known in the art.

A test antibody competes with the reference antibody for binding to the antigen if an excess of one antibody (e.g., 1, 5, 10, 20, 100, 1000, 10000 or 100000-fold) inhibits binding of the other antibody, e.g., by at least 50%, 75%, 90%, 95% or 99%, as measured in a competitive binding assay.

Unless otherwise indicated competition is determined using a competitive ELISA assay as described above.

The term “binding affinity” is herein used as a measure of the strength of a non-covalent interaction between two molecules, e.g. an antibody, or fragment thereof, and an antigen.

The term “binding affinity” is used to describe monovalent interactions.

Binding affinity between two molecules, e.g. an antibody, or fragment thereof, and an antigen, through a monovalent interaction may be quantified by determining the equilibrium dissociation constant (K_{D}). K_{D} can be determined by measurement of the kinetics of complex formation and dissociation, e.g. by the Surface Plasmon Resonance (SPR) method or the Isothermal Titration Calorimetry (ITC) method. The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ.

Following the above definition, binding affinities associated with different molecular interactions, such as comparison of the binding affinity of different antibodies for a given antigen, may be compared by comparison of the K_{D} values for the individual antibody/antigen complexes.

The value of the dissociation constant can be determined directly by well-known methods. Standard assays to evaluate the binding ability of ligands such as antibodies towards targets are known in the art and include, for example, ELISAs, Western blots, RIAs, and flow cytometry analysis. The binding kinetics and binding affinity of the antibody also can be assessed by standard assays known in the art, such as SPR. Preferably, however, isothermal titration calorimetry (ITC) may be used to measure affinities for an antibody/target interaction as well as to derive thermodynamic parameters for the interaction.

A competitive binding assay can be conducted in which the binding of the antibody to the target is compared to the binding of the target by another ligand of that target, such as another antibody.

An antibody of the invention may have a K_{D} for its target of 1×10⁻⁴M or less, 1×10⁻⁵M or less, 1×10⁻⁶M or less, 1×10⁻⁷M or less, 1×10⁻⁸M or less, or 1×10⁻⁹M or less, or 1×10⁻¹⁰M or less, 1×10⁻¹¹M or less, 1×10⁻¹²M or less, 1×10⁻¹³M or less or 1×10⁻¹⁴M or less.

The K_{D} of an antibody of the invention may be less than 100 μM such as less than 10 μM, such as less than 1 μM, such as less than 0.9 μM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM.

In one such embodiment the antibody is a bispecific antibody comprising an anti-FX arm with a K_{D} towards FX of less than 100 μM such as less than 1 0 μM, such as less than 1 μM, such as less than 0.9 μM, such as less than 0.8 μM, such as less than 0.7 μM, such as less than 0.6 μM, such as less than 0.5 μM, such as less than 0.4 μM, such as less than 0.3 μM, such as less than 0.2 μM, such as less than 0.1 μM, such as less than 0.09 μM, such as less than 0.08 μM, such as less than 0.07 μM, such as less than 0.06 μM, such as less than 0.05 μM, such as less than 0.04 μM, such as less than 0.03 μM, such as less than 0.02 μM, such as less than 0.01 μM, such as less than 9 nM, such as less than 8 nM, such as less than 7 nM, such as less than 6 nM, such as less than 5 nM, such as less than 4 nM, such as less than 3 nM, such as less than 2 nM, such as less than 1 nM such as less than 0.5 nM.

The antibodies and antibody fragment thereof as described herein may be combined with other antibodies and antibody fragments known in the art creating bispecific, trispecific or multispecific antibody molecules. Compounds mimicking FVIII cofactor function have previously been created using other FIX/IXa and FX/Xa binding domains, which may potentially each substitute for the FIX/IXa and/or FX/Xa binding domains described herein. It is thus clear that the FIX/IXa and FX/Xa binding domains of the present invention are of separate interest as individual molecules, as well as “intermediates” as part of a bi-, tri- or multispecific antibody comprising at least one FIX/IXa and/or FX/Xa binding domain.

The activity of procoagulant antibodies including bi-, tri and multispecific antibodies may be determined by methods known in the art. Standard assays include whole blood-Thrombin-Generation Test (TGT), measuring of clotting time by thrombelastography (TEG) and FXa generation assays.

### Identity

The term “identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al. SIAM J. Applied Math. 1988; 48:1073. Preferred methods for determining identity are designed to give the largest match between the sequences tested. Methods of determining identity are described in publicly available computer programs. Preferred computer program methods for determining identity between two sequences include the GCG program package, including GAP (Devereux et al. Nucl. Acid. Res. 1984; 12:387); Genetics Computer Group, University of Wisconsin, Madison, Wis.), BLASTP, BLASTN, and FASTA (Altschul et al. J. Mol. Biol. 1990; 215:403-410). The BLASTX program is publicly available from the National Center for Biotechnology Information (NCBI) and other sources (BLAST Manual, Altschul et al. NCB/NLM/NIH Bethesda, Md. 20894; Altschul et al. supra). The well-known Smith Waterman algorithm may also be used to determine identity.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10times the gap opening penalty), as well as a comparison matrix such as PAM 250 or BLOSUM 62 are used in conjunction with the algorithm. A standard comparison matrix (see Dayhoff et al. 1978; Atlas of Protein Sequence and Structure, vol. 5, supp.3 for the PAM 250 comparison matrix; Henikoff et al. PNAS 1992; 89:10915-10919 for the BLOSUM 62 comparison matrix) is also used by the algorithm. Preferred parameters for a peptide sequence comparison include the following: Algorithm: Needleman et al. J. Mol. Biol. 1970; 48:443-453; Comparison matrix: BLOSUM 62 from Henikoff et al. PNAS 1992; 89:10915-10919; Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

The term “similarity” is a related concept, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity is 25% and the percent similarity would be 75% ((fraction ( 15/20))). Therefore, in cases where there are conservative substitutions, the degree of similarity between two polypeptides will be higher than the percent identity between those two polypeptides.

### Pharmaceutical Formulations

In another aspect, the present invention provides compositions and formulations comprising compounds of the invention, such as the antibodies described herein. For example, the invention provides a pharmaceutical composition that comprises one or more antibodies of the invention, formulated together with a pharmaceutically acceptable carrier.

Accordingly, one object of the invention is to provide a pharmaceutical formulation comprising such an antibody which is present in a concentration from 0.25 mg/ml to 250 mg/ml, and wherein said formulation has a pH from 2.0 to 10.0. The formulation may further comprise one or more of a buffer system, a preservative, a tonicity agent, a chelating agent, a stabilizer, or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19^{th} edition, 1995.

In one embodiment the pharmaceutical formulation is an aqueous formulation. Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, to which the physician or the patient adds solvents and/or diluents prior to use.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

### Administration

A compound of the invention, such as an antibody, may be administered parenterally, such as intravenously, such as intramuscularly, such as subcutaneously. Alternatively, an antibody of the invention may be administered via a non-parenteral route, such as periorally or topically. An antibody of the invention may be administered prophylactically. An antibody of the invention may be administered therapeutically (on demand).

### Dosages

The dose of the compounds to be delivered may be from about 0.01 mg to 500 mg of the compound per day, preferably from about 0.1 mg to 250 mg per day, and more preferably from about 0.5 mg to about 250 mg per day, per week, per second week or per month as loading and maintenance doses, depending on the severity of the condition. A suitable dose may also be adjusted for a particular compound based on the properties of that compound, including its in vivo half-life or mean residence time and its biological activity. For example, compounds to be delivered could in one embodiment be administered once weekly, or in another embodiment once every second week or in another embodiment one monthly and in either of said embodiments in a dose of for example 0.25, 0.5, 1, 1.5, 2, 2.5, 3, 3.5, 4, 4.5, 5, 5.5, 6, 6.5, 7, 7.5, 8, 8.5, 9, 9.5 or 10 mg per kg body weight.

The compositions containing the compounds as disclosed herein can be administered for prophylactic and/or in some embodiments therapeutic treatments. In therapeutic applications, compositions are administered to a subject already suffering from a disease, such as any bleeding disorder as described above, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease or injury as well as the weight and general state of the subject.

### EMBODIMENTS

In one embodiment an antibody of the invention is capable of binding an epitope comprising residue H256 of FIX (SEQ ID NO:1) or the activated form thereof (FIXa).

In one embodiment an antibody of the invention is capable of binding an epitope comprising residue H257 of FIX (SEQ ID NO:1) or the activated form thereof FIXa.

In one embodiment an antibody of the invention is capable of binding an epitope comprising residue N258 of FIX (SEQ ID NO:1) or the activated form thereof (FIXa).

In one embodiment an antibody of the invention is capable of binding an epitope comprising residue K293 of FIX (SEQ ID NO:1) or the activated form thereof (FIXa).

In one embodiment an antibody of the invention is capable of binding an epitope comprising residue K301 of FIX (SEQ ID NO:1) or the activated form thereof (FIXa).

In one embodiment an antibody of the invention is capable of binding an epitope comprising residue D332 of FIX (SEQ ID NO:1) or the activated form thereof (FIXa).

In one embodiment an antibody of the invention is capable of binding an epitope comprising residue R333 of FIX (SEQ ID NO:1) or the activated form thereof (FIXa).

In one embodiment an antibody of the invention is capable of binding an epitope comprising residue A334 of FIX (SEQ ID NO:1) or the activated form thereof (FIXa).

In one embodiment an antibody of the invention is capable of binding an epitope comprising residue T335 of FIX (SEQ ID NO:1) or the activated form thereof (FIXa).

In one embodiment an antibody of the invention is capable of binding an epitope comprising residue L337 of FIX (SEQ ID NO:1) or the activated form thereof (FIXa).

In one embodiment an antibody of the invention is capable of binding an epitope comprising residue R338 of FIX (SEQ ID NO:1) or the activated form thereof (FIXa).

In one embodiment an antibody of the invention is capable of binding an epitope comprising residue S339 of FIX (SEQ ID NO:1) or the activated form thereof (FIXa).

In one embodiment an antibody of the invention is capable of binding an epitope comprising residue T340 of FIX (SEQ ID NO:1) or the activated form thereof (FIXa).

In one embodiment an antibody of the invention is capable of binding an epitope comprising residue K341 of FIX (SEQ ID NO:1) or the activated form thereof (FIXa).

In one embodiment an antibody of the invention is capable of binding an epitope comprising residue T343 of FIX (SEQ ID NO:1) or the activated form thereof (FIXa).

In one embodiment an antibody of the invention is capable of binding an epitope comprising residue N346 of FIX (SEQ ID NO:1) or the activated form thereof (FIXa).

In one embodiment an antibody of the invention is capable of binding an epitope comprising residue R403 of FIX (SEQ ID NO:1) or the activated form thereof (FIXa).

In one embodiment an antibody of the invention is capable of binding an epitope comprising residue Y404 of FIX (SEQ ID NO:1) or the activated form thereof (FIXa).

In one embodiment an antibody of the invention is capable of binding an epitope comprising residue N406 of FIX (SEQ ID NO:1) or the activated form thereof (FIXa).

In one embodiment an antibody of the invention is capable of binding an epitope comprising residue W407 of FIX (SEQ ID NO:1) or the activated form thereof (FIXa).

In one embodiment an antibody of the invention is capable of binding an epitope comprising residue E410 of FIX (SEQ ID NO:1) or the activated form thereof (FIXa).

In one embodiment an antibody of the invention is capable of binding an epitope comprising residue K411 of FIX (SEQ ID NO:1) or the activated form thereof (FIXa).

In one embodiment an antibody of the invention is capable of binding an epitope comprising residues L337, R338, S339, T340, K341, and T343 of FIX (SEQ ID NO:1) or the activated form thereof (FIXa).

In one embodiment an antibody of the invention is capable of binding an epitope comprising residues K301, D332, R333, A334, T335, R338, and N346 of FIX (SEQ ID NO:1) or the activated form thereof (FIXa).

In one embodiment an antibody of the invention is capable of binding an epitope comprising residues H256, H257, N258, K293, R403, Y404, N406, W407, E410, and K411 of FIX (SEQ ID NO:1) or the activated form thereof (FIXa).

In one embodiment an antibody of the invention is capable of binding FIX (SEQ ID NO:1) or the activated form thereof (FIXa), wherein the antibody competes with Fab7236 for binding to FIX.

In one embodiment an antibody of the invention is capable of binding FIX (SEQ ID NO:1) or the activated form thereof (FIXa), wherein the antibody competes with Fab7237 for binding to FIX.

In one embodiment an antibody of the invention is capable of binding FIX (SEQ ID NO:1) or the activated form thereof (FIXa), wherein the antibody competes with Fab7238 for binding to FIX.

In one embodiment an antibody of the invention binds FIX (SEQ ID NO:1) or the activated form thereof (FIXa), wherein the antibody belongs to the same “bin” as Fab7236.

In one embodiment an antibody of the invention binds FIX (SEQ ID NO:1) or the activated form thereof (FIXa), wherein the antibody belongs to the same “bin” as Fab7237.

In one embodiment an antibody of the invention binds FIX (SEQ ID NO:1) or the activated form thereof (FIXa), wherein the antibody belongs to the same “bin” as Fab7238.

In one embodiment an antibody of the invention is capable of binding FX (SEQ ID NO:2) or the activated form thereof FXa, wherein the antibody competes with an antibody comprising the CDRs of mAb1-6723.

In one embodiment an antibody of the invention is capable of binding FX/FXa, wherein the antibody comprises the variable domains of mAb1-6723 according to SEQ ID NO:21 and SEQ ID NO:22.

In one embodiment an antibody of the invention is capable of specifically binding FX/FXa, wherein the antibody comprises the CDRs of mAb1-6723 according to SEQ ID NO:21 and SEQ ID NO:22.

In one embodiment an antibody of the invention is capable of binding FX/FXa, wherein the antibody belongs to the same “bin” as a Fab of comprising the variable domains of mAb1-6723 according to SEQ ID NO:21 and SEQ ID NO:22.

In one embodiment an antibody of the invention is capable of specifically binding FX/FXa, wherein the antibody belongs to the same “bin” as mAb1-6723 according to SEQ ID NO:21 and SEQ ID NO:22.

In one embodiment an antibody of the invention is capable of specifically binding FX/FXa, wherein the antibody belongs to the same “bin” as an antibody or Fab comprising the antigen binding domain according to SEQ ID NO:21 and SEQ ID NO:22.

In one embodiment an antibody of the invention is capable of binding FX/FXa, wherein the antibody competes with an antibody comprising the CDRs of mAb1-1371.

In one embodiment an antibody of the invention is capable of binding FX/FXa, wherein the antibody comprises the variable domains of mAb1-1371 according to SEQ ID NO:65 and SEQ ID NO:66.

In one embodiment an antibody of the invention is capable of binding FX/FXa, wherein the antibody comprises the CDRs of mAb1-1371 according to SEQ ID NO:65 and SEQ ID NO:66.

In one embodiment an antibody of the invention is capable of binding FX/FXa, wherein the antibody belongs to the same “bin”, herein referred to as “Bin A”, as a Fab of comprising the variable domains of mAb1-1371 according to SEQ ID NO:65 and SEQ ID NO:66.

In one embodiment an antibody of the invention is capable of binding FX/FXa, wherein the antibody belongs to the same “bin” as mAb1-1371 according to SEQ ID NO:65 and SEQ ID NO:66.

In one embodiment an antibody of the invention is capable of binding FX/FXa, wherein the antibody belongs to the same “bin” as an antibody or Fab comprising the antigen binding domain according to SEQ ID NO:65 and SEQ ID NO:66.

In one embodiment an antibody of the invention is capable of specifically binding FX/FXa, wherein the antibody competes with an antibody comprising the CDRs of mAb1-1376, mAb1-6705, mAb1-7388 or mAb1-7563. Such antibodies are herein referred to as belonging to Bin B.

In one embodiment an antibody of the invention is capable of binding FX/FXa, wherein the antibody comprises the variable domains of mAb1-1376, mAb1-6705, mAb1-7388 or mAb1-7563 as identified by SEQ ID NO:67 and 68, SEQ ID NO:23 and 24, SEQ ID NO:39 and 40, and SEQ ID NO:59 and 60, respectively.

In one embodiment an antibody of the invention is capable of binding FX/FXa, wherein the antibody comprises the CDRs of mAb1-1376, mAb1-6705, mAb1-7388 or mAb1-7563 as identified by SEQ ID NO:67 and 68, SEQ ID NO:23 and 24, SEQ ID NO:39 and 40, and SEQ ID NO:59 and 60, respectively.

In one embodiment an antibody of the invention is capable of binding FX/FXa, wherein the antibody belongs to the same “bin” as a Fab of comprising the variable domains of mAb1-1376, mAb1-6705, mAb1-7388 or mAb1-7563 as identified by SEQ ID NO:67 and 68, SEQ ID NO:23 and 24, SEQ ID NO:39 and 40, and SEQ ID NO:59 and 60, respectively.

In one embodiment an antibody of the invention capable of binding FX/FXa, wherein the antibody belongs to the same “bin” as mAb1-1376, mAb1-6705, mAb1-7388 or mAb1-7563 as identified by SEQ ID NO:67 and 68, SEQ ID NO:23 and 24, SEQ ID NO:39 and 40, and SEQ ID NO:59 and 60, respectively.

In one embodiment an antibody of the invention is capable of binding FX/FXa, wherein the antibody belongs to the same “bin” as an antibody or Fab comprising the antigen binding domain according to SEQ ID NO:67 and 68, SEQ ID NO:23 and 24, SEQ ID NO:39 and 40, or SEQ ID NO:59 and 60.

In one embodiment an antibody of the invention is capable of binding FX/FXa, wherein the antibody comprises the CDRs or variable domains of an antibody selected from the group consisting of: mAb1-6723, 1-6716, 1-6721, 1-6730, 1-6731, 1-6737, 1-6754, 1-7378, 1-7413, 1-7424, 1-7466, 1-7481, 1-7483 and mAb1-7591.

In one embodiment an antibody of the invention is capable of binding FX/FXa, wherein the antibody comprises the CDRs or variable domains of an antibody selected from the group consisting of: mAb1-6723, 1-6716, 1-6721, 1-6730, 1-6731, 1-6737, 1-6754, 1-7378, 1-7413, 1-7424, 1-7466, 1-7481, 1-7483, 1-7591, 1-7388, 1-7563, 1-7462 and mAb1-7571.

In one embodiment an antibody of the invention is capable of binding FX/FXa, wherein the antibody belongs to the same “bin” as a mAb selected from the group of mAbs comprising the variables sequences or the CDRs thereof selected from: SEQ ID NO:21 and 22, SEQ ID NO:25 and 26, SEQ ID NO:27 and 28, SEQ ID NO:29 and 30, SEQ ID NO:31 and 32, SEQ ID NO:33 and 34, SEQ ID NO:35 and 36, SEQ ID NO:37 and 38, SEQ ID NO:39 and 40, SEQ ID NO:41 and 42, SEQ ID NO:43 and 44, SEQ ID NO:45 and 46, SEQ ID NO:51 and 52, SEQ ID NO:53 and 54, SEQ ID NO:55 and 56, SEQ ID NO:57 and 58, SEQ ID NO:59 and 60, SEQ ID NO:61 and 62, and SEQ ID NO:63 and 64. This “bin” of antibodies have herein been referred to as Bin C and exemplified with a large number of individual antibodies, such as mAb1-6723, 1-6716, 1-6721, 1-6730, 1-6731, 1-6737, 1-6754, 1-7378, 1-7413, 1-7424, 1-7466, 1-7481, 1-7483, 1-7591, 1-7388, 1-7563, 1-7462 and mAb1-7571.

In one embodiment an antibody of the invention is capable of binding FX/FXa, wherein the antibody competes for binding to FX, FX zymogen or FXa with a reference antibody selected from the group of antibodies consisting of mAbs comprising the variables sequences or the CDRs thereof selected from: SEQ ID NO:21 and 22, SEQ ID NO:25 and 26, SEQ ID NO:27 and 28, SEQ ID NO:29 and 30, SEQ ID NO:31 and 32, SEQ ID NO:33 and 34, SEQ ID NO:35 and 36, SEQ ID NO:37 and 38, SEQ ID NO:41 and 42, SEQ ID NO:43 and 44, SEQ ID NO:53 and 54, SEQ ID NO:55 and 56, SEQ ID NO:57 and 58, and SEQ ID NO:63 and 64.

In one embodiment the antibody or antigen-binding fragment thereof according to the invention competes for binding to FX/FXa with an antigen-binding fragment comprising the CDRs of SEQ ID NO:21 and 22, SEQ ID NO:25 and 26, SEQ ID NO:27 and 28, SEQ ID NO:29 and 30, SEQ ID NO:31 and 32, SEQ ID NO:33 and 34, SEQ ID NO:35 and 36, SEQ ID NO:37 and 38, SEQ ID NO:41 and 42, SEQ ID NO:43 and 44, SEQ ID NO:53 and 54, SEQ ID NO:55 and 56, SEQ ID NO:57 and 58, or SEQ ID NO:63 and 64.

In one embodiment an antibody of the invention is capable of binding FX/FXa, wherein the antibody competes with an antibody comprising the CDRs of mAb1-7447, 1-7441, 1-7571 or 1-7462. These are herein referred to as antibodies of Bin D.

In one embodiment an antibody of the invention is capable of binding FX/FXa, wherein the antibody comprises the variable domains of mAb1-7447, 1-7441, 1-7571 or 1-7462 according to SEQ ID NO:47 and 48, SEQ ID NO:45 and 46, SEQ ID NO:51 and 53 or SEQ ID NO:61 and 62, respectively.

In one embodiment an antibody of the invention is capable of binding FX/FXa, wherein the antibody comprises the variable domains of mAb1-7447 or 1-7441, according to SEQ ID NO:47 and 48, SEQ ID NO:45 and 46, respectively.

In one embodiment an antibody of the invention is capable of binding FX/FXa, wherein the antibody comprises the CDRs of mAb1-7447,1-7441, 1-7571 or 1-7462 according to SEQ ID NO:47 and 48, SEQ ID NO:45 and 46, SEQ ID NO:51 and 53 or SEQ ID NO:61 and 62, respectively.

In one embodiment an antibody of the invention is capable of binding FX/FXa according to SEQ ID NO:2, wherein the antibody belongs to the same “bin” as a Fab comprising the variable domains of mAb1-7447, 1-7441, 1-7571 or mAb1-7462 according to SEQ ID NO:47 and 48, SEQ ID NO:45 and 46, SEQ ID NO:51 and 53 or SEQ ID NO:61 and 62, respectively.

In one embodiment an antibody of the invention is capable of binding FX/FXa, wherein the antibody belongs to the same “bin” as mAb1-7447, 1-7441, 1-7571 or mAb1-7462 according to SEQ ID NO:47 and 48, SEQ ID NO:45 and 46, SEQ ID NO:51 and 53 or SEQ ID NO:61 and 62 respectively.

In one embodiment an antibody of the invention is capable of binding FX/FXa, wherein the antibody belongs to the same “bin” as an antibody or Fab comprising the antigen-binding domain according to SEQ ID NO:47 and 48, SEQ ID NO:45 and 46, SEQ ID NO:51 and 53 or SEQ ID NO:61 and 62.

In one embodiment an antibody of the invention is an antibody according to any of the previous embodiments, wherein the antibody specifically binds the FX zymogen.

In such embodiment the antibody specifically binds the FX zymogen according to amino acid residues 1-139, 143-448 of SEQ ID NO:2.

In one embodiment an antibody of the invention is an antibody according to any of the previous embodiments, wherein the antibody binds FX.

In one embodiment an antibody of the invention is an antibody according to any of the previous embodiments, wherein the antibody binds FXa.

In one such embodiment the antibody specifically binds FXa according to amino acid residues 1-139, 195-448 of SEQ ID NO:2.

In one embodiment the antibody is a monospecific antibody. In one embodiment the antibody is a multispecific antibody. In one such embodiment the antibody is a bispecific antibody. In one such embodiment the bispecific antibody is capable of binding to FIX or the activated form thereof (FIXa) and FX/FXa. In one such embodiment the bispecific antibody is capable of specifically binding to FIX/FIXa and FX/FXa.

In one embodiment the antibody is a bispecific antibody capable of binding FIX/FIXa and FX/FXa, wherein the FIX/FIXa binding domain is derived from an antibody of Bin 1 and the FX/FXa binding domain is derived from an antibody of Bin A.

In one embodiment the antibody is a bispecific antibody binding FIX/FIXa and FX/FXa, wherein the FIX/FIXa binding domain is derived from an antibody of Bin 2 and the FX/FXa binding domain is derived from an antibody of Bin A.

In one embodiment the antibody is a bispecific antibody binding FIX/FIXa and FX/FXa, wherein the FIX/FIXa binding domain is derived from an antibody of Bin 2 and the FX/FXa binding domain is derived from an antibody of Bin B.

In one embodiment the antibody is a bispecific antibody binding FIX/FIXa and FX/FXa, wherein the FIX/FIXa binding domain is derived from an antibody of Bin 2 and the FX/FXa binding domain is derived from an antibody of Bin B.

In one embodiment the antibody is a bispecific antibody binding FIX/FIXa and FX/FXa, wherein the FIX/FIXa binding domain is derived from an antibody of Bin 1 and the FX/FXa binding domain is derived from an antibody of Bin C or D.

In one embodiment the antibody is a bispecific antibody binding FIX/FIXa and FX/FXa, wherein the binding domains are derived from the mAb pairs consisting of: mAb1-1371/mAb1-1307, mAb1-6705/mAb1-1307, mAb1-1371/mAb0-1886, mAb1-7441/mAb0-1886, mAb1-7447/mAb0-1886, mAb1-7481/mAb0-1886, mAb1-1371/mAb0-1998, mAb1-6716/mAb0-1998, mAb1-6723/mAb0-1998, mAb1-6730/mAb0-1998, mAb1-6731/mAb0-1998, mAb1-6737/mAb0-1998, mAb1-6754/mAb0-1998, mAb1-7378/mAb0-1998, mAb1-7441/mAb0-1998, mAb1-7447/mAb0-1998, mAb1-7481/mAb0-1998, mAb1-1371/mAb1-4707, mAb1-6705/mAb1-4707, mAb1-1371/mAb1-4071, mAb1-7441/mAb1-5788, mAb1-7447/mAb1-5788, mAb1-7481/mAb1-5788, mAb1-1371/mAb1-4857, mAb1-6716/mAb1-4857, mAb1-6723/mAb1-4857, mAb1-6730/mAb1-4857, mAb1-6731/mAb1-4857, mAb1-6737/mAb1-4857, mAb1-6754/mAb1-4857, mAb1-7378/mAb1-4857, mAb1-7441/mAb1-4857, mAb1-7447/mAb1-4857 or mAb1-7481/mAb1-4857.

In one embodiment a bispecific antibody of the invention comprises an antibody arm binding to FX and an antibody arm binding to FIX/FIXa. In one such embodiment the antibody arm binding to FX binds to an epitope comprising one or more residues in the activation peptide of FX and the antibody arm binding FIX/FIXa binds to an epitope comprising one or more residues in the FIX protease domain.

In one embodiment an antibody of the invention is a multispecific antibody, such as bi- or trispecific antibody.

In one embodiment an antibody of the invention is in IgG format such as full length IgG4.

In one embodiment an antibody of the invention is a chemical conjugate of two antibody fragments, such as a conjugate of two Fab fragments or scFv fragments, or combinations thereof.

In one embodiment an antibody of the invention is a human or humanised antibody.

In one embodiment the antibodies disclosed herein are intermediates for use in the manufacture of a bispecific antibody.

In one embodiment the invention includes antibodies competing for binding to FIX/FIXa with the antibodies disclosed herein.

An antibody of the current invention can be used to treat a subject with a coagulopathy and in particular haemophilia A. Thus, the invention also relates to the use of a monoclonal antibody, that is capable of binding the protease domain of FIX/FIXa, for the treatment of a subject in need thereof; as well as use of said antibody for the manufacture of a medicament for the treatment of a subject in need thereof. Furthermore, the invention includes a method of treating a subject in need thereof with a monoclonal antibody that is capable of binding to the protease domain of FIX/FIXa.

In one embodiment an antibody of the invention is capable of binding FIXa with a higher affinity than that with which it binds FIX.

In one embodiment an antibody of the invention is capable of increasing the enzymatic activity of FIXa towards FX.

In one such embodiment an antibody of the invention is capable of increasing the enzymatic activity of FIXa towards FX as measured in a FXa generation assay using monovalent one-armed antibodies as described herein.

In one embodiment an antibody of the invention is capable of increasing the enzymatic activity of FIXa towards FX as measured in a FXa generation assay using bivalent antibodies as described herein.

In one embodiment an antibody of the invention is not the anti-FIX antibody CLB-FIX 13 as described in Rohlena et al. (2003) J. Biol. Chem. 278(11):9394-9401. In one embodiment an antibody of the invention is not the anti-FIX antibody HIX-1 (IgG1 murine) (Merck KGaA, SigmaAldrich). In one embodiment an antibody of the invention is not the anti-FIX antibody AHIX-5041 (IgG1) (Haematologic Technologies, Inc.).

In one embodiment an antibody of the invention has reduced immunogenicity as compared to procoagulant antibodies of the art.

In one embodiment a bispecific antibody or antigen-binding fragment thereof comprises a first antigen-binding site recognizing FIX (SEQ ID NO:1) or the activated form thereof (FIXa), and a second antigen-binding site recognizing FX (SEQ ID NO:2) or the activated form thereof (FXa) wherein
a) the first antigen-binding site comprises the following CDR sequences:

(SEQ ID NO: 189)
V_{H}-CDR1: DYAMH
(SEQ ID NO: 190)
V_{H}-CDR2: GISWRGDIIGYVDSVKG
(SEQ ID NO: 191)
V_{H}-CDR3: SYGSGSFYNAFDS
(SEQ ID NO: 192)
V_{L}-CDR1: RASQSISSWLA
(SEQ ID NO: 193)
V_{L}-CDR2: KASRLDR
(SEQ ID NO: 194)
V_{L}-CDR3: LEYSSYIRT

and
b) the second antigen-binding site comprises the following CDR sequences:

(SEQ ID NO: 195)
V_{H}-CDR1: TSWIV
(SEQ ID NO: 196)
V_{H}-CDR2: MIDPSDSFTSYSPSFQG
(SEQ ID NO: 197)
V_{H}-CDR3: LHYYHSEEFDV
(SEQ ID NO: 198)
V_{L}-CDR1: RASQSVSSSYLA
(SEQ ID NO: 199)
V_{L}-CDR2: GASSRAR
(SEQ ID NO: 200)
V_{L}-CDR3: QQFGSSRLFT

In one embodiment a bispecific antibody or antigen-binding fragment thereof comprises a first antigen-binding site recognizing FIX (SEQ ID NO:1) or the activated form thereof (FIXa), and a second antigen-binding site recognizing FX (SEQ ID NO:2) or the activated form thereof (FXa) wherein
a) the first antigen-binding site comprises the following CDR sequences:

(SEQ ID NO: 189)
V_{H}-CDR1: DYAMH
(SEQ ID NO: 190)
V_{H}-CDR2: GISWRGDIIGYVDSVKG
(SEQ ID NO: 191)
V_{H}-CDR3: SYGSGSFYNAFDS
(SEQ ID NO: 201)
V_{L}-CDR1: RASQSISSWLA
(SEQ ID NO: 193)
V_{L}-CDR2: KASRLDR
(SEQ ID NO: 194)
V_{L}-CDR3: LEYSSYIRT

and
b) the second antigen-binding site comprises the following CDR sequences:

(SEQ ID NO: 195)
V_{H}-CDR1: TSWIV
(SEQ ID NO: 196)
V_{H}-CDR2: MIDPSDSFTSYSPSFQG
(SEQ ID NO: 197)
V_{H}-CDR3: LHYYHSEEFDV
(SEQ ID NO: 198)
V_{L}-CDR1: RASQSVSSSYLA
(SEQ ID NO: 202)
V_{L}-CDR2: GASSRTR
(SEQ ID NO: 200)
V_{L}-CDR3: QQFGSSRLFT

The invention is further described by the following embodiments:
1. An antibody or antigen-binding fragment thereof capable of binding to Factor IX (FIX) according to SEQ ID NO:1 or the activated form hereof (FIXa).
2. The antibody or antigen-binding fragment thereof according to embodiment 1, wherein the antibody or antigen-binding fragment thereof is part of “Bin1”.
3. The antibody or antigen-binding fragment thereof according to embodiment 1, wherein the antibody or antigen-binding fragment thereof competes with a reference antibody wherein the reference antibody comprises
   a. a heavy chain variable domain identified by SEQ ID NO:15 and a light chain variable domain identified by SEQ ID NO:16 or
   b. a heavy chain variable domain identified by SEQ ID NO:19 and a light chain variable domain identified by SEQ ID NO:20.
4. The antibody according to the previous embodiment, wherein the reference antibody is a Fab.
5. The antibody or antigen-binding fragment thereof according to any of the previous embodiments, wherein the antibody or antigen-binding fragment thereof comprises
   a. a heavy chain variable domain at least 90% identical to the sequence identified by SEQ ID NO:15 and a light chain variable domain at least 90% identical to the sequence identified by SEQ ID NO:16,
   b. a heavy chain variable domain at least 90% identical to the sequence identified by SEQ ID NO:19 and a light chain variable domain at least 90% identical to the sequence identified by SEQ ID NO:20,
   c. a heavy chain variable domain at least 90% identical to the sequence identified by SEQ ID NO:69 and a light chain variable domain at least 90% identical to the sequence identified by SEQ ID NO:70,
   d. a heavy chain variable domain at least 90% identical to the sequence identified by SEQ ID NO:71 and a light chain variable domain at least 90% identical to the sequence identified by SEQ ID NO:72,
   e. a heavy chain variable domain at least 90% identical to the sequence identified by SEQ ID NO:73 and a light chain variable domain at least 90% identical to the sequence identified by SEQ ID NO:74,
   f. a heavy chain variable domain at least 90% identical to the sequence identified by SEQ ID NO:83 and a light chain variable domain at least 90% identical to the sequence identified by SEQ ID NO:84,
   g. a heavy chain variable domain at least 90% identical to the sequence identified by SEQ ID NO:81 and a light chain variable domain at least 90% identical to the sequence identified by SEQ ID NO:82,
   h. a heavy chain variable domain at least 90% identical to the sequence identified by SEQ ID NO:75 and a light chain variable domain at least 90% identical to the sequence identified by SEQ ID NO:76,
   i. a heavy chain variable domain at least 90% identical to the sequence identified by SEQ ID NO:77 and a light chain variable domain at least 90% identical to the sequence identified by SEQ ID NO:78, or
   j. heavy chain variable domain at least 90% identical to the sequence identified by SEQ ID NO:177 and a light chain variable domain at least 90% identical to the sequence identified by SEQ ID NO:178.
6. The antibody or antigen-binding fragment thereof according to embodiment 5, wherein in the heavy chain variable domain is at least 92, 94, 96 or 98% identical to the identified SEQ IDs.
7. The antibody or antigen-binding fragment thereof according to embodiment 5, wherein in the light chain variable domain is at least 92, 94, 96 or 98% identical to the identified SEQ IDs.
8. The antibody or antigen-binding fragment thereof according to embodiment 6 and 7, wherein both the heavy chain variable domain and the light chain variable domain are at least 92, 94, 96 or 98% identical to the identified SEQ IDs.
9. The antibody or antigen-binding fragment thereof according to any of the previous embodiments, wherein the antibody or antigen-binding fragment thereof comprises
   a.
      i. three heavy chain CDR sequences with at most 10 amino acid changes compared to the CDR sequences of the heavy chain variable domain identified by SEQ ID NO:15 and
      ii. three light chain CDR sequences with at most 10 amino acid changes compared to the CDR sequences of the light chain variable domain identified by SEQ ID NO:16,
   b.
      i. three heavy chain CDR sequences with at most 10 amino acid changes compared to the CDR sequences of the heavy chain variable domain identified by SEQ ID NO:19 and
      ii. three light chain CDR sequences with at most 10 amino acid changes compared to the CDR sequences of the light chain variable domain identified by SEQ ID NO:20
   c.
      i. three heavy chain CDR sequences with at most 10 amino acid changes compared to the CDR sequences of the heavy chain variable domain identified by SEQ ID NO:69 and
      ii. three light chain CDR sequences with at most 10 amino acid changes compared to the CDR sequences of the light chain variable domain identified by SEQ ID NO:70,
   d.
      i. three heavy chain CDR sequences with at most 10 amino acid changes compared to the CDR sequences of the heavy chain variable domain identified by SEQ ID NO:71 and
      ii. three light chain CDR sequences with at most 10 amino acid changes compared to the CDR sequences of the light chain variable domain identified by SEQ ID NO:72,
   e.
      i. three heavy chain CDR sequences with at most 10 amino acid changes compared to the CDR sequences of the heavy chain variable domain identified by SEQ ID NO:73 and
      ii. three light chain CDR sequences with at most 10 amino acid changes compared to the CDR sequences of the light chain variable domain identified by SEQ ID NO:74,
   f.
      i. three heavy chain CDR sequences with at most 10 amino acid changes compared to the CDR sequences of the heavy chain variable domain identified by SEQ ID NO:83 and
      ii. three light chain CDR sequences with at most 10 amino acid changes compared to the CDR sequences of the light chain variable domain identified by SEQ ID NO:84,
   g.
      i. three heavy chain CDR sequences with at most 10 amino acid changes compared to the CDR sequences of the heavy chain variable domain identified by SEQ ID NO:81 and
      ii. three light chain CDR sequences with at most 10 amino acid changes compared to the CDR sequences of the light chain variable domain identified by SEQ ID NO:82,
   h.
      i. three heavy chain CDR sequences with at most 10 amino acid changes compared to the CDR sequences of the heavy chain variable domain identified by SEQ ID NO:75 and
      ii. three light chain CDR sequences with at most 10 amino acid changes compared to the CDR sequences of the light chain variable domain identified by SEQ ID NO:76 or
   i.
      i. three heavy chain CDR sequences with at most 10 amino acid changes compared to the CDR sequences of the heavy chain variable domain identified by SEQ ID NO:77 and
      ii. three light chain CDR sequences with at most 10 amino acid changes compared to the CDR sequences of the light chain variable domain identified by SEQ ID NO:78 or
   j.
      i. three heavy chain CDR sequences with at most 10 amino acid changes compared to the CDR sequences of the heavy chain variable domain identified by SEQ ID NO:177 and
      ii. three light chain CDR sequences with at most 10 amino acid changes compared to the CDR sequences of the light chain variable domain identified by SEQ ID NO:178.
10. The antibody or antigen-binding fragment thereof according to embodiment 9, wherein the three heavy chain CDR sequences have at most 9, such as 8, such as 7 or such as 6 amino acid changes compared to the CDRs of the identified SEQ IDs.
11. The antibody or antigen-binding fragment thereof according to embodiment 9, wherein the three heavy chain CDR sequences have at most 5, such as 4, such as 3, such as 2 or at most 1 amino acid changes compared to the CDRs of the identified SEQ IDs.
12. The antibody or antigen-binding fragment thereof according to embodiment 9, wherein the three light chain CDR sequences have at most 9, such as 8, such as 7 or such as 6 amino acid changes compared to the CDRs of the identified SEQ IDs.
13. The antibody or antigen-binding fragment thereof according to embodiment 9, wherein the three light chain CDR sequences have at most 5, such as 4, such as 3, such as 2 or at most 1 amino acid changes compared to the CDRs of the identified SEQ IDs.
14. The antibody or antigen-binding fragment thereof according to any of the previous embodiments, wherein the antibody or antigen-binding fragment thereof comprises
   a. the CDR sequences of the heavy chain variable domain identified by SEQ ID NO:15 and the CDR sequences of the light chain variable domain identified by SEQ ID NO:16, or
   b. the CDR sequences of the heavy chain variable domain identified by SEQ ID NO:19 and the CDR sequences of the light chain variable domain identified by SEQ ID NO:20, or
   c. the CDR sequences of the heavy chain variable domain identified by SEQ ID NO:3 and the CDR sequences of the light chain variable domain identified by SEQ ID NO:4, or
   d. the CDR sequences of the heavy chain variable domain identified by SEQ ID NO:109 and the CDR sequences of the light chain variable domain identified by SEQ ID NO:110, or
   e. the CDR sequences of the heavy chain variable domain identified by SEQ ID NO:153 and the CDR sequences of the light chain variable domain identified by SEQ ID NO:154, or
   f. the CDR sequences of the heavy chain variable domain identified by SEQ ID NO:171 and the CDR sequences of the light chain variable domain identified by SEQ ID NO:172, or
   g. the CDR sequences of the heavy chain variable domain identified by SEQ ID NO:177 and the CDR sequences of the light chain variable domain identified by SEQ ID NO:178, or
   h. the CDR sequences of the heavy chain variable domain identified by SEQ ID NO:187 and the CDR sequences of the light chain variable domain identified by SEQ ID NO:188.
15. The antibody or antigen-binding fragment thereof according to any of the previous embodiments, wherein the antibody or antigen-binding fragment thereof comprises
   a. a heavy chain variable domain identified by SEQ ID NO:15 and a light chain variable domain identified by SEQ ID NO:16, or
   b. a heavy chain variable domain identified by SEQ ID NO:19 and a light chain variable domain identified by SEQ ID NO:20, or
   c. a heavy chain variable domain identified by SEQ ID NO:3 and a light chain variable domain identified by SEQ ID NO:4, or
   d. a heavy chain variable domain identified by SEQ ID NO:109 and a light chain variable domain identified by SEQ ID NO:110, or
   e. a heavy chain variable domain identified by SEQ ID NO:153 and a light chain variable domain identified by SEQ ID NO:154, or
   f. a heavy chain variable domain identified by SEQ ID NO:171 and a light chain variable domain identified by SEQ ID NO:172, or
   g. a heavy chain variable domain identified by SEQ ID NO:177 and a light chain variable domain identified by SEQ ID NO:178, or
   h. a heavy chain variable domain identified by SEQ ID NO:187 and a light chain variable domain identified by SEQ ID NO:188.
16. The antibody or antigen-binding fragment thereof according to any of the previous embodiments, wherein the antibody or antigen-binding fragment thereof is capable of binding an epitope comprising one or more of the amino acid residues L337, R338, S339, T340, K341 and T343 of SEQ ID NO:1.
17. The antibody or antigen-binding fragment thereof according to any of the previous embodiments, wherein the antibody or antigen-binding fragment thereof is capable of binding an epitope comprising amino acid residue R338 of SEQ ID NO:1.
18. The antibody or antigen-binding fragment thereof according to any of the previous embodiments, wherein the antibody or antigen-binding fragment thereof is capable of binding an epitope comprising amino acid residues R338 and K341 of SEQ ID NO:1.
19. The antibody or antigen-binding fragment thereof according to any of the previous embodiments, wherein the antibody or antigen-binding fragment thereof is capable of binding an epitope comprising two or three of the amino acid residues L337, R338, S339, T340, K341 and T343 of SEQ ID NO:1.
20. The antibody or antigen-binding fragment thereof according to any of the previous embodiments, wherein the antibody or antigen-binding fragment thereof is capable of binding an epitope comprising four or five of the amino acid residues L337, R338, S339, T340, K341 and T343 of SEQ ID NO:1.
21. The antibody or antigen-binding fragment thereof according to any of the previous embodiments, wherein the antibody or antigen-binding fragment thereof is capable of binding an epitope comprising
   a. R338, S339, T340, K341 and T343,
   b. L337, S339, T340, K341 and T343,
   c. L337, R338, T340, K341 and T343,
   d. L337, R338, S339, K341 and T343,
   e. L337, R338, S339, T340 and T343 or
   f. L337, R338, S339, T340 and K341 of SEQ ID NO:1.
22. The antibody or antigen-binding fragment thereof according to any of the previous embodiments, wherein the antibody or antigen-binding fragment thereof is capable of binding an epitope comprising amino acid residues L337, R338, S339, T340, K341 and T343 of SEQ ID NO:1.
23. The antibody or antigen-binding fragment thereof according to any of the previous embodiments, wherein the antibody or antigen-binding fragment thereof is capable of binding an epitope comprising one or more of the amino acid residues K301, D332, R333, A334, T335, R338, and N346 of SEQ ID NO:1.
24. The antibody or antigen-binding fragment thereof according to any of the previous embodiments, wherein the antibody or antigen-binding fragment thereof is capable of binding an epitope comprising amino acid residues L337, R338, S339, T340, K341 and T343 of SEQ ID NO:1.
25. The antibody or antigen-binding fragment thereof according to any of the previous embodiments, wherein the antibody or antigen-binding fragment thereof is capable of binding an epitope comprising two or three of the amino acid residues K301, D332, R333, A334, T335, R338, and N346 of SEQ ID NO:1.
26. The antibody or antigen-binding fragment thereof according to any of the previous embodiments, wherein the antibody or antigen-binding fragment thereof is capable of binding an epitope comprising four or five of the amino acid residues K301, D332, R333, A334, T335, R338, and N346 of SEQ ID NO:1.
27. The antibody or antigen-binding fragment thereof according to any of the previous embodiments, wherein the antibody or antigen-binding fragment thereof is capable of binding an epitope comprising five or six of the amino acid residues K301, D332, R333, A334, T335, R338, and N346 of SEQ ID NO:1.
28. The antibody or antigen-binding fragment thereof according to any of the previous embodiments, wherein the antibody or antigen-binding fragment thereof is capable of binding an epitope comprising
   a. D332, R333, A334, T335, R338 and N346
   b. K301, R333, A334, T335, R338 and N346
   c. K301, D332, A334, T335, R338 and N346
   d. K301, D332, R333, T335, R338 and N346
   e. K301, D332, R333, A334, R338 and N346
   f. K301, D332, R333, A334, T335 and N346 or
   g. K301, D332, R333, A334, T335 and R338
   of SEQ ID NO:1.
29. The antibody or antigen-binding fragment thereof according to any of the previous embodiments, wherein the antibody or antigen-binding fragment thereof is capable of binding an epitope comprising the amino acid residues D332, R333, L337 and R338 of SEQ ID NO:1.
30. The antibody or antigen-binding fragment thereof according to any of the previous embodiments, wherein the antibody or antigen-binding fragment thereof is capable of binding an epitope comprising the amino acid residues K301, D332, R333, A334, T335, R338, and N346 of SEQ ID NO:1.
31. The antibody or antigen-binding fragment thereof according to embodiment 1, wherein the antibody or antigen-binding fragment thereof belongs to “Bin2”.
32. The antibody or antigen-binding fragment thereof according to embodiment 1, wherein the antibody or antigen-binding fragment thereof competes with a reference antibody wherein the reference antibody comprises a heavy chain variable domain identified by the sequence of SEQ ID NO:17 and a light chain variable domain identified by the sequence of SEQ ID NO:18.
33. The antibody according to the previous embodiment, wherein the reference antibody is a Fab.
34. The antibody or antigen-binding fragment thereof according to any of the previous embodiments, wherein the antibody or antigen-binding fragment thereof comprises
   a. a heavy chain variable domain at least 90% identical to the sequence identified by SEQ ID NO:17 and a light chain variable domain at least 90% identical to the sequence identified by SEQ ID NO:18.
   b. a heavy chain variable domain at least 90% identical to the sequence identified by SEQ ID NO:85 and a light chain variable domain at least 90% identical to the sequence identified by SEQ ID NO:86 or
   c. a heavy chain variable domain at least 90% identical to the sequence identified by SEQ ID NO:79 and a light chain variable domain at least 90% identical to the sequence identified by SEQ ID NO:80.
35. The antibody or antigen-binding fragment thereof according to embodiment 34, wherein in the heavy chain variable domain is at least 92, 94, 96 or 98% identical to the identified SEQ IDs.
36. The antibody or antigen-binding fragment thereof according to embodiment 34, wherein in the light chain variable domain is at least 92, 94, 96 or 98% identical to the identified SEQ IDs.
37. The antibody or antigen-binding fragment thereof according to embodiment 35 and 36, wherein both the heavy chain variable domain and the light chain variable domain are at least 92, 94, 96 or 98% identical to the identified SEQ IDs.
38. The antibody or antigen-binding fragment thereof according to any of the previous embodiments, wherein the antibody or antigen-binding fragment thereof comprises
   a.
      i. three heavy chain CDR sequences with at most 10 amino acid changes compared to the CDR sequences of the heavy chain variable domain identified by SEQ ID NO:17 and
      ii. three light chain CDR sequences with at most 10 amino acid changes compared to the CDR sequences of the light chain variable domain identified by SEQ ID NO:18,
   b.
      i. three heavy chain CDR sequences with at most 10 amino acid changes compared to the CDR sequences of the heavy chain variable domain identified by SEQ ID NO:85 and
      ii. three light chain CDR sequences with at most 10 amino acid changes compared to the CDR sequences of the light chain variable domain identified by SEQ ID NO:86 or
   c.
      i. three heavy chain CDR sequences with at most 10 amino acid changes compared to the CDR sequences of the heavy chain variable domain identified by SEQ ID NO:79 and
      ii. three light chain CDR sequences with at most 10 amino acid changes compared to the CDR sequences of the light chain variable domain identified by SEQ ID NO:80.
39. The antibody or antigen-binding fragment thereof according to embodiment 38, wherein the three heavy chain CDR sequences have at most 9, such as 8, such as 7 or such as 6 amino acid changes compared to the CDRs of the identified SEQ IDs.
40. The antibody or antigen-binding fragment thereof according to embodiment 38, wherein the three heavy chain CDR sequences have at most 5, such as 4, such as 3, such as 2 or at most 1 amino acid changes compared to the CDRs of the identified SEQ IDs.
41. The antibody or antigen-binding fragment thereof according to embodiment 38, wherein the three light chain CDR sequences have at most 9, such as 8, such as 7 or such as 6 amino acid changes compared to the CDRs of the identified SEQ IDs.
42. The antibody or antigen-binding fragment thereof according to embodiment 38, wherein the three light chain CDR sequences have at most 5, such as 4, such as 3, such as 2 or at most 1 amino acid changes compared to the CDRs of the identified SEQ IDs.
43. The antibody or antigen-binding fragment thereof according to any of the previous embodiments, wherein the antibody or antigen-binding fragment thereof comprises
   a. the CDR sequences of the heavy chain variable domain identified by SEQ ID NO:17 and
   the CDR sequences of the light chain variable domain identified by SEQ ID NO:18.
44. The antibody or antigen-binding fragment thereof according to any of the previous embodiments, wherein the antibody or antigen-binding fragment thereof comprises
   a. a heavy chain variable domain identified by SEQ ID NO:17 and a light chain variable domain identified by SEQ ID NO:18.
45. The antibody or antigen-binding fragment thereof according to any of the previous embodiments, wherein the antibody or antigen-binding fragment thereof is capable of binding an epitope comprising one or more of the amino acid residues H256, H257, N258, K293, R403, Y404, N406, W407, E410, and K411 of SEQ ID NO:1.
46. The antibody or antigen-binding fragment thereof according to any of the previous embodiments, wherein the antibody or antigen-binding fragment thereof is capable of binding an epitope comprising the amino acid residues H256, H257, N258, K293, R403, Y404, N406, W407, E410, and K411 SEQ ID NO:1.
47. The antibody or antigen-binding fragment thereof according to any of the previous embodiments, wherein the antibody or antigen-binding fragment thereof is capable of binding an epitope comprising two, three or four of the amino acid residues H256, H257, N258, K293, R403, Y404, N406, W407, E410, and K411 of SEQ ID NO:1.
48. The antibody or antigen-binding fragment thereof according to any of the previous embodiments, wherein the antibody or antigen-binding fragment thereof is capable of binding an epitope comprising five, six or seven of the amino acid residues H256, H257, N258, K293, R403, Y404, N406, W407, E410, and K411 of SEQ ID NO:1.
49. The antibody or antigen-binding fragment thereof according to any of the previous embodiments, wherein the antibody or antigen-binding fragment thereof is capable of binding an epitope comprising eight, nine or ten of the amino acid residues H256, H257, N258, K293, R403, Y404, N406, W407, E410, and K411 of SEQ ID NO:1.
50. The antibody or antigen-binding fragment thereof according to any of the previous embodiments, wherein the antibody or antigen-binding fragment thereof is capable of binding an epitope comprising the amino acid residues
   a. H257, N258, K293, R403, Y404, N406, W407, E410 and K411,
   b. H256, N258, K293, R403, Y404, N406, W407, E410 and K411,
   c. H256, H257, K293, R403, Y404, N406, W407, E410 and K411,
   d. H256, H257, N258, R403, Y404, N406, W407, E410 and K411,
   e. H256, H257, N258, K293, Y404, N406, W407, E410 and K411,
   f. H256, H257, N258, K293, R403, N406, W407, E410 and K411,
   g. H256, H257, N258, K293, R403, Y404, W407, E410 and K411,
   h. H256, H257, N258, K293, R403, Y404, N406, E410 and K411,
   i. H256, H257, N258, K293, R403, Y404, N406, W407 and K411 or
   j. H256, H257, N258, K293, R403, Y404, N406, W407 and E410 of SEQ ID NO:1.
51. The antibody or antigen-binding fragment thereof according to any of the previous embodiments, wherein the antibody or antigen-binding fragment thereof is capable of binding an epitope comprising the amino acid residues H256, H257, N258, K293, R403, Y404, N406, W407, E410, and K411 of SEQ ID NO:1.
52. The antibody or antigen-binding fragment thereof according to any of the previous embodiments, wherein the antibody or antigen-binding fragment thereof is capable of binding an epitope comprising the amino acid residues H257, K293 and N406 of SEQ ID NO:1.
53. The antibody or antigen-binding fragment thereof according to any one of the previous embodiments wherein the antibody is a procoagulant antibody.
54. The antibody or antigen-binding fragment thereof according to any one of the previous embodiments wherein the antibody or antigen-binding fragment thereof is capable of increasing the procoagulant activity of FIXa.
55. The antibody or antigen-binding fragment thereof according to any one of the previous embodiments wherein the antibody is capable of increasing the enzymatic activity of FIXa towards FX.
56. The antibody or antigen-binding fragment thereof according to any one of the previous embodiments wherein the antibody is capable of functionally substituting for FVIII and/or FVIIIa.
57. An antibody or antigen-binding fragment thereof capable of binding to FX (SEQ ID NO:2) or the activated form thereof (FXa).
58. The antibody or antigen-binding fragment thereof according to embodiment 57, wherein the antibody or antigen-binding fragment thereof is part of “Bin A”.
59. The antibody or antigen-binding fragment thereof according to embodiment 57, wherein the antibody or antigen-binding fragment thereof competes with a reference antibody wherein the reference antibody comprises a heavy chain variable domain identified by SEQ ID NO:65 and a light chain variable domain identified by SEQ ID NO:66.
60. The antibody according to the previous embodiment, wherein the reference antibody is a Fab.
61. The antibody or antigen-binding fragment thereof according to any of the previous embodiments, wherein the antibody or antigen-binding fragment thereof is capable of binding an epitope comprising one or more of the amino acid residues H101, E103, R113, T116, L117, A118, T127, S227, E228, F229, Y230, E266, R287, L303, P304, E305, L419, K420, D423, R424, M426, K427 and T428 of FX/FXa.
62. The antibody or antigen-binding fragment thereof according to any of the previous embodiments, wherein the antibody or antigen-binding fragment thereof is capable of binding an epitope comprising the amino acid residues H101, E103, R113, T116, L117, A118, T127, S227, E228, F229, Y230, E266, R287, P304, L303, P304, E305, L419, K420, D423, R424, M426, K427 and T428 of FX/FXa.
63. The antibody or antigen-binding fragment thereof according to any of the previous embodiments, wherein the antibody or antigen-binding fragment thereof is capable of binding an epitope comprising one or more of the amino acid residues R113, Y230, K420 D423, R424 and K427 of FX/FXa.
64. The antibody or antigen-binding fragment thereof according to any of the previous embodiments, wherein the antibody or antigen-binding fragment thereof is capable of binding an epitope comprising the amino acid residues R113, Y230, K420, D423, R424 and K427 of FX/FXa.
65. The antibody or antigen-binding fragment thereof according to any of the previous embodiments, wherein the antibody or antigen-binding fragment thereof comprises a heavy chain variable domain at least 90% identical to the sequence identified by SEQ ID NO:65 and a light chain variable domain at least 90% identical to the sequence identified by SEQ ID NO:66.
66. The antibody or antigen-binding fragment thereof according to embodiment 65, wherein in the heavy chain variable domain is at least 92, 94, 96 or 98% identical to the identified SEQ IDs.
67. The antibody or antigen-binding fragment thereof according to embodiment 65, wherein in the light chain variable domain is at least 92, 94, 96 or 98% identical to the identified SEQ IDs.
68. The antibody or antigen-binding fragment thereof according to embodiment 66 and 67, wherein both the heavy chain variable domain and the light chain variable domain are at least 92, 94, 96 or 98% identical to the identified SEQ IDs.
69. The antibody or antigen-binding fragment thereof according to any of the previous embodiments, wherein the antibody or antigen-binding fragment thereof comprises
   a. three heavy chain CDR sequences with at most 10 amino acid changes compared to the CDR sequences of the heavy chain variable domain identified by SEQ ID NO:65 and
   b. three light chain CDR sequences with at most 10 amino acid changes compared to the CDR sequences of the light chain variable domain identified by SEQ ID NO:66.
70. The antibody or antigen-binding fragment thereof according to embodiment 69, wherein the three heavy chain CDR sequences have at most 9, such as 8, such as 7 or such as 6 amino acid changes compared to the CDRs of the identified SEQ IDs.
71. The antibody or antigen-binding fragment thereof according to embodiment 69, wherein the three heavy chain CDR sequences have at most 5, such as 4, such as 3, such as 2 or at most 1 amino acid changes compared to the CDRs of the identified SEQ IDs.
72. The antibody or antigen-binding fragment thereof according to embodiment 69, wherein the three light chain CDR sequences have at most 9, such as 8, such as 7 or such as 6 amino acid changes compared to the CDRs of the identified SEQ IDs.
73. The antibody or antigen-binding fragment thereof according to embodiment 69, wherein the three light chain CDR sequences have at most 5, such as 4, such as 3, such as 2 or at most 1 amino acid changes compared to the CDRs of the identified SEQ IDs.
74. The antibody or antigen-binding fragment thereof according to any of the previous embodiments, wherein the antibody or antigen-binding fragment thereof comprises the CDR sequences of the heavy chain variable domain identified by SEQ ID NO:65 and the CDR sequences of the light chain variable domain identified by SEQ ID NO:66.
75. The antibody or antigen-binding fragment thereof according to any of the previous embodiments, wherein the antibody or antigen-binding fragment thereof comprises a heavy chain variable domain identified by SEQ ID NO:65 and a light chain variable domain identified by SEQ ID NO:66.
76. The antibody or antigen-binding fragment thereof according to embodiment 57, wherein the antibody or antigen-binding fragment thereof is part of “Bin B”.
77. The antibody or antigen-binding fragment thereof according to embodiment 57, wherein the antibody or antigen-binding fragment thereof competes with a reference antibody wherein the reference antibody comprises a heavy chain variable domain identified by SEQ ID NO:67 and a light chain variable domain identified by SEQ ID NO:68.
78. The antibody or antigen-binding fragment thereof according to the previous embodiment, wherein the reference antibody is a Fab.
79. The antibody or antigen-binding fragment thereof according to any of the previous embodiments, wherein the antibody or antigen-binding fragment thereof comprises
   a. a heavy chain variable domain at least 90% identical to the sequence identified by SEQ ID NO:67 and a light chain variable domain at least 90% identical to the sequence identified by SEQ ID NO:68,
   b. a heavy chain variable domain at least 90% identical to the sequence identified by SEQ ID NO:23 and a light chain variable domain at least 90% identical to the sequence identified by SEQ ID NO:24,
   c. a heavy chain variable domain at least 90% identical to the sequence identified by SEQ ID NO:39 and a light chain variable domain at least 90% identical to the sequence identified by SEQ ID NO:40 or
   d. a heavy chain variable domain at least 90% identical to the sequence identified by SEQ ID NO:59 and a light chain variable domain at least 90% identical to the sequence identified by SEQ ID NO:60.
80. The antibody or antigen-binding fragment thereof according to embodiment 79, wherein in the heavy chain variable domain is at least 92, 94, 96 or 98% identical to the identified SEQ IDs.
81. The antibody or antigen-binding fragment thereof according to embodiment 79, wherein in the light chain variable domain is at least 92, 94, 96 or 98% identical to the identified SEQ IDs.
82. The antibody or antigen-binding fragment thereof according to embodiment 80 and 81, wherein both the heavy chain variable domain and the light chain variable domain are at least 92, 94, 96 or 98% identical to the identified SEQ IDs.
83. The antibody or antigen-binding fragment thereof according to any of the previous embodiments, wherein the antibody or antigen-binding fragment thereof comprises
   a.
      i. three heavy chain CDR sequences with at most 10 amino acid changes compared to the CDR sequences of the heavy chain variable domain identified by SEQ ID NO:67 and
      ii. three light chain CDR sequences with at most 10 amino acid changes compared to the CDR sequences of the light chain variable domain identified by SEQ ID NO:68,
   b.
      i. three heavy chain CDR sequences with at most 10 amino acid changes compared to the CDR sequences of the heavy chain variable domain identified by SEQ ID NO:23 and
      ii. three light chain CDR sequences with at most 10 amino acid changes compared to the CDR sequences of the light chain variable domain identified by SEQ ID NO:24,
   c.
      i. three heavy chain CDR sequences with at most 10 amino acid changes compared to the CDR sequences of the heavy chain variable domain identified by SEQ ID NO:39 and
      ii. three light chain CDR sequences with at most 10 amino acid changes compared to the CDR sequences of the light chain variable domain identified by SEQ ID NO:40 or
   d.
      i. three heavy chain CDR sequences with at most 10 amino acid changes compared to the CDR sequences of the heavy chain variable domain identified by SEQ ID NO:59 and
      ii. three light chain CDR sequences with at most 10 amino acid changes compared to the CDR sequences of the light chain variable domain identified by SEQ ID NO:60.
84. The antibody or antigen-binding fragment thereof according to embodiment 83, wherein the three heavy chain CDR sequences have at most 9, such as 8, such as 7 or such as 6 amino acid changes compared to the CDRs of the identified SEQ IDs.
85. The antibody or antigen-binding fragment thereof according to embodiment 83, wherein the three heavy chain CDR sequences have at most 5, such as 4, such as 3, such as 2 or at most 1 amino acid changes compared to the CDRs of the identified SEQ IDs.
86. The antibody or antigen-binding fragment thereof according to embodiment 83, wherein the three light chain CDR sequences have at most 9, such as 8, such as 7 or such as 6 amino acid changes compared to the CDRs of the identified SEQ IDs.
87. The antibody or antigen-binding fragment thereof according to embodiment 83, wherein the three light chain CDR sequences have at most 5, such as 4, such as 3, such as 2 or at most 1 amino acid changes compared to the CDRs of the identified SEQ IDs.
88. The antibody or antigen-binding fragment thereof according to any of the previous embodiments, wherein the antibody or antigen-binding fragment thereof comprises
   a. the CDR sequences of the heavy chain variable domain identified by SEQ ID NO:67 and the CDR sequences of the light chain variable domain identified by SEQ ID NO:68,
   b. the CDR sequences of the heavy chain variable domain identified by SEQ ID NO:23 and the CDR sequences of the light chain variable domain identified by SEQ ID NO:24,
   c. the CDR sequences of the heavy chain variable domain identified by SEQ ID NO:39 and the CDR sequences of the light chain variable domain identified by SEQ ID NO:40 or
   d. the CDR sequences of the heavy chain variable domain identified by SEQ ID NO:59 and the CDR sequences of the light chain variable domain identified by SEQ ID NO:60.
89. The antibody or antigen-binding fragment thereof according to any of the previous embodiments, wherein the antibody or antigen-binding fragment thereof comprises
   a. a heavy chain variable domain identified by SEQ ID NO:67 and a light chain variable domain identified by SEQ ID NO:68,
   b. a heavy chain variable domain identified by SEQ ID NO:23 and a light chain variable domain identified by SEQ ID NO:24,
   c. a heavy chain variable domain identified by SEQ ID NO:39 and a light chain variable domain identified by SEQ ID NO:40 or
   d. a heavy chain variable domain identified by SEQ ID NO:59 and a light chain variable domain identified by SEQ ID NO:60.
90. The antibody or antigen-binding fragment thereof according to embodiment 57, wherein the antibody or antigen-binding fragment thereof is part of “Bin C”.
91. The antibody or antigen-binding fragment thereof according to embodiment 57, wherein the antibody or antigen-binding fragment thereof competes with a reference antibody wherein the reference antibody comprises a heavy chain variable domain identified by SEQ ID NO:21 and a light chain variable domain identified by SEQ ID NO:22.
92. The antibody or antigen-binding fragment thereof according to the previous embodiment, wherein the reference antibody is a Fab.
93. The antibody or antigen-binding fragment thereof according to any of the previous embodiments, wherein the antibody or antigen-binding fragment thereof comprises
   a. a heavy chain variable domain at least 90% identical to the sequence identified by SEQ ID NO:21 and a light chain variable domain at least 90% identical to the sequence identified by SEQ ID NO:22,
   b. a heavy chain variable domain at least 90% identical to the sequence identified by SEQ ID NO:25 and a light chain variable domain at least 90% identical to the sequence identified by SEQ ID NO:26.
   c. a heavy chain variable domain at least 90% identical to the sequence identified by SEQ ID NO:27 and a light chain variable domain at least 90% identical to the sequence identified by SEQ ID NO:28,
   d. a heavy chain variable domain at least 90% identical to the sequence identified by SEQ ID NO:29 and a light chain variable domain at least 90% identical to the sequence identified by SEQ ID NO:30,
   e. a heavy chain variable domain at least 90% identical to the sequence identified by SEQ ID NO:31 and a light chain variable domain at least 90% identical to the sequence identified by SEQ ID NO:32,
   f. a heavy chain variable domain at least 90% identical to the sequence identified by SEQ ID NO:33 and a light chain variable domain at least 90% identical to the sequence identified by SEQ ID NO:34.
   g. a heavy chain variable domain at least 90% identical to the sequence identified by SEQ ID NO:35 and a light chain variable domain at least 90% identical to the sequence identified by SEQ ID NO:36,
   h. a heavy chain variable domain at least 90% identical to the sequence identified by SEQ ID NO:37 and a light chain variable domain at least 90% identical to the sequence identified by SEQ ID NO:38,
   i. a heavy chain variable domain at least 90% identical to the sequence identified by SEQ ID NO:39 and a light chain variable domain at least 90% identical to the sequence identified by SEQ ID NO:40,
   j. a heavy chain variable domain at least 90% identical to the sequence identified by SEQ ID NO:41 and a light chain variable domain at least 90% identical to the sequence identified by SEQ ID NO:42,
   k. a heavy chain variable domain at least 90% identical to the sequence identified by SEQ ID NO:43 and a light chain variable domain at least 90% identical to the sequence identified by SEQ ID NO:44,
   l. a heavy chain variable domain at least 90% identical to the sequence a heavy chain variable domain at least 90% identical to the sequence identified by SEQ ID NO:51 and a light chain variable domain at least 90% identical to the sequence identified by SEQ ID NO:52,
   m. a heavy chain variable domain at least 90% identical to the sequence identified by SEQ ID NO:53 and a light chain variable domain at least 90% identical to the sequence identified by SEQ ID NO:54,
   n. a heavy chain variable domain at least 90% identical to the sequence identified by SEQ ID NO:55 and a light chain variable domain at least 90% identical to the sequence identified by SEQ ID NO:56,
   o. a heavy chain variable domain at least 90% identical to the sequence identified by SEQ ID NO:57 and a light chain variable domain at least 90% identical to the sequence identified by SEQ ID NO:58,
   p. a heavy chain variable domain at least 90% identical to the sequence identified by SEQ ID NO:59 and a light chain variable domain at least 90% identical to the sequence identified by SEQ ID NO:60,
   q. a heavy chain variable domain at least 90% identical to the sequence identified by SEQ ID NO:61 and a light chain variable domain at least 90% identical to the sequence identified by SEQ ID NO:62 or
   r. a heavy chain variable domain at least 90% identical to the sequence identified by SEQ ID NO:63 and a light chain variable domain at least 90% identical to the sequence identified by SEQ ID NO:64.
94. The antibody or antigen-binding fragment thereof according to embodiment 93, wherein in the heavy chain variable domain is at least 92, 94, 96 or 98% identical to the identified SEQ IDs.
95. The antibody or antigen-binding fragment thereof according to embodiment 93, wherein in the light chain variable domain is at least 92, 94, 96 or 98% identical to the identified SEQ IDs.
96. The antibody or antigen-binding fragment thereof according to embodiment 94 and 95, wherein both the heavy chain variable domain and the light chain variable domain are at least 92, 94, 96 or 98% identical to the identified SEQ IDs.
97. The antibody or antigen-binding fragment thereof according to any of the previous embodiments, wherein the antibody or antigen-binding fragment thereof comprises
   a.
      i. three heavy chain CDR sequences with at most 10 amino acid changes compared to the CDR sequences of the heavy chain variable domain identified by SEQ ID NO:21 and
      ii. three light chain CDR sequences with at most 10 amino acid changes compared to the CDR sequences of the light chain variable domain identified by SEQ ID NO:22,
   b.
      i. three heavy chain CDR sequences with at most 10 amino acid changes compared to the CDR sequences of the heavy chain variable domain identified by SEQ ID NO:25 and
      ii. three light chain CDR sequences with at most 10 amino acid changes compared to the CDR sequences of the light chain variable domain identified by SEQ ID NO:26,
   c.
      i. three heavy chain CDR sequences with at most 10 amino acid changes compared to the CDR sequences of the heavy chain variable domain identified by SEQ ID NO:27 and
      ii. three light chain CDR sequences with at most 10 amino acid changes compared to the CDR sequences of the light chain variable domain identified by SEQ ID NO:28 or
   d.
      i. three heavy chain CDR sequences with at most 10 amino acid changes compared to the CDR sequences of the heavy chain variable domain identified by SEQ ID NO:29 and
      ii. three light chain CDR sequences with at most 10 amino acid changes compared to the CDR sequences of the light chain variable domain identified by SEQ ID NO:30,
   e.
      i. three heavy chain CDR sequences with at most 10 amino acid changes compared to the CDR sequences of the heavy chain variable domain identified by SEQ ID NO:31 and
      ii. three light chain CDR sequences with at most 10 amino acid changes compared to the CDR sequences of the light chain variable domain identified by SEQ ID NO:32,
   f.
      i. three heavy chain CDR sequences with at most 10 amino acid changes compared to the CDR sequences of the heavy chain variable domain identified by SEQ ID NO:33 and
      ii. three light chain CDR sequences with at most 10 amino acid changes compared to the CDR sequences of the light chain variable domain identified by SEQ ID NO:34,
   g.
      i. three heavy chain CDR sequences with at most 10 amino acid changes compared to the CDR sequences of the heavy chain variable domain identified by SEQ ID NO:35 and
      ii. three light chain CDR sequences with at most 10 amino acid changes compared to the CDR sequences of the light chain variable domain identified by SEQ ID NO:36 or
   h.
      i. three heavy chain CDR sequences with at most 10 amino acid changes compared to the CDR sequences of the heavy chain variable domain identified by SEQ ID NO:37 and
      ii. three light chain CDR sequences with at most 10 amino acid changes compared to the CDR sequences of the light chain variable domain identified by SEQ ID NO:38
   i.
      i. three heavy chain CDR sequences with at most 10 amino acid changes compared to the CDR sequences of the heavy chain variable domain identified by SEQ ID NO:39 and
      ii. three light chain CDR sequences with at most 10 amino acid changes compared to the CDR sequences of the light chain variable domain identified by SEQ ID NO:40,
   j.
      i. three heavy chain CDR sequences with at most 10 amino acid changes compared to the CDR sequences of the heavy chain variable domain identified by SEQ ID NO:41 and
      ii. three light chain CDR sequences with at most 10 amino acid changes compared to the CDR sequences of the light chain variable domain identified by SEQ ID NO:42,
   k.
      i. three heavy chain CDR sequences with at most 10 amino acid changes compared to the CDR sequences of the heavy chain variable domain identified by SEQ ID NO:43 and
      ii. three light chain CDR sequences with at most 10 amino acid changes compared to the CDR sequences of the light chain variable domain identified by SEQ ID NO:44 or
   l.
   m.
      i. three heavy chain CDR sequences with at most 10 amino acid changes compared to the CDR sequences of the heavy chain variable domain identified by SEQ ID NO:51 and
      ii. three light chain CDR sequences with at most 10 amino acid changes compared to the CDR sequences of the light chain variable domain identified by SEQ ID NO:52 or
   n.
      i. three heavy chain CDR sequences with at most 10 amino acid changes compared to the CDR sequences of the heavy chain variable domain identified by SEQ ID NO:53 and
         three light chain CDR sequences with at most 10 amino acid changes compared to the CDR sequences of the light chain variable domain identified by SEQ ID NO:54
   o.
      i. three heavy chain CDR sequences with at most 10 amino acid changes compared to the CDR sequences of the heavy chain variable domain identified by SEQ ID NO:55 and
      ii. three light chain CDR sequences with at most 10 amino acid changes compared to the CDR sequences of the light chain variable domain identified by SEQ ID NO:56
   p.
      i. three heavy chain CDR sequences with at most 10 amino acid changes compared to the CDR sequences of the heavy chain variable domain identified by SEQ ID NO:57 and
      ii. three light chain CDR sequences with at most 10 amino acid changes compared to the CDR sequences of the light chain variable domain identified by SEQ ID NO:58,
   q.
      i. three heavy chain CDR sequences with at most 10 amino acid changes compared to the CDR sequences of the heavy chain variable domain identified by SEQ ID NO:59 and
      ii. three light chain CDR sequences with at most 10 amino acid changes compared to the CDR sequences of the light chain variable domain identified by SEQ ID NO:60,
   r.
      i. three heavy chain CDR sequences with at most 10 amino acid changes compared to the CDR sequences of the heavy chain variable domain identified by SEQ ID NO:61 and
      ii. three light chain CDR sequences with at most 10 amino acid changes compared to the CDR sequences of the light chain variable domain identified by SEQ ID NO:62 or
   s.
      i. three heavy chain CDR sequences with at most 10 amino acid changes compared to the CDR sequences of the heavy chain variable domain identified by SEQ ID NO:63 and
      ii. three light chain CDR sequences with at most 10 amino acid changes compared to the CDR sequences of the light chain variable domain identified by SEQ ID NO:64.
98. The antibody or antigen-binding fragment thereof according to embodiment 97, wherein the three heavy chain CDR sequences have at most 9, such as 8, such as 7 or such as 6 amino acid changes compared to the CDRs of the identified SEQ IDs.
99. The antibody or antigen-binding fragment thereof according to embodiment 97, wherein the three heavy chain CDR sequences have at most 5, such as 4, such as 3, such as 2 or at most 1 amino acid changes compared to the CDRs of the identified SEQ IDs.
100. The antibody or antigen-binding fragment thereof according to embodiment 97, wherein the three light chain CDR sequences have at most 9, such as 8, such as 7 or such as 6 amino acid changes compared to the CDRs of the identified SEQ IDs.
101. The antibody or antigen-binding fragment thereof according to embodiment 97, wherein the three light chain CDR sequences have at most 5, such as 4, such as 3, such as 2 or at most 1 amino acid changes compared to the CDRs of the identified SEQ IDs.
102. The antibody or antigen-binding fragment thereof according to any of the previous embodiments, wherein the antibody or antigen-binding fragment thereof comprises
   a. the CDR sequences of the heavy chain variable domain identified by SEQ ID NO:21 and the CDR sequences of the light chain variable domain identified by SEQ ID NO:22,
   b. the CDR sequences of the heavy chain variable domain identified by SEQ ID NO:25 and the CDR sequences of the light chain variable domain identified by SEQ ID NO:26,
   c. the CDR sequences of the heavy chain variable domain identified by SEQ ID NO:27 and the CDR sequences of the light chain variable domain identified by SEQ ID NO:28,
   d. the CDR sequences of the heavy chain variable domain identified by SEQ ID NO:29 and the CDR sequences of the light chain variable domain identified by SEQ ID NO:30,
   e. the CDR sequences of the heavy chain variable domain identified by SEQ ID NO:31 and the CDR sequences of the light chain variable domain identified by SEQ ID NO:32,
   f. the CDR sequences of the heavy chain variable domain identified by SEQ ID NO:33 and the CDR sequences of the light chain variable domain identified by SEQ ID NO:34,
   g. the CDR sequences of the heavy chain variable domain identified by SEQ ID NO:35 and the CDR sequences of the light chain variable domain identified by SEQ ID NO:36,
   h. the CDR sequences of the heavy chain variable domain identified by SEQ ID NO:37 and the CDR sequences of the light chain variable domain identified by SEQ ID NO:38,
   i. the CDR sequences of the heavy chain variable domain identified by SEQ ID NO:39 and the CDR sequences of the light chain variable domain identified by SEQ ID NO:40,
   j. the CDR sequences of the heavy chain variable domain identified by SEQ ID NO:41 and the CDR sequences of the light chain variable domain identified by SEQ ID NO:42,
   k. the CDR sequences of the heavy chain variable domain identified by SEQ ID NO:43 and the CDR sequences of the light chain variable domain identified by SEQ ID NO:44,
   l. the CDR sequences of the heavy chain variable domain identified by SEQ ID NO:51 and the CDR sequences of the light chain variable domain identified by SEQ ID NO:52,
   m. the CDR sequences of the heavy chain variable domain identified by SEQ ID NO:53 and the CDR sequences of the light chain variable domain identified by SEQ ID NO:54,
   n. the CDR sequences of the heavy chain variable domain identified by SEQ ID NO:55 and the CDR sequences of the light chain variable domain identified by SEQ ID NO:56,
   o. the CDR sequences of the heavy chain variable domain identified by SEQ ID NO:57 and the CDR sequences of the light chain variable domain identified by SEQ ID NO:58,
   p. the CDR sequences of the heavy chain variable domain identified by SEQ ID NO:59 and the CDR sequences of the light chain variable domain identified by SEQ ID NO:60,
   q. the CDR sequences of the heavy chain variable domain identified by SEQ ID NO:61 and the CDR sequences of the light chain variable domain identified by SEQ ID NO:62 or
   r. the CDR sequences of the heavy chain variable domain identified by SEQ ID NO:63 and the CDR sequences of the light chain variable domain identified by SEQ ID NO:64.
103. The antibody or antigen-binding fragment thereof according to any of the previous embodiments, wherein the antibody or antigen-binding fragment thereof comprises a heavy chain variable domain and a light chain variable domain identified by
   a. SEQ ID NO:21 and 22,
   b. SEQ ID NO:25 and 26,
   c. SEQ ID NO:27 and 28,
   d. SEQ ID NO:29 and 30,
   e. SEQ ID NO:31 and 32,
   f. SEQ ID NO:33 and 34,
   g. SEQ ID NO:35 and 36,
   h. SEQ ID NO:37 and 38,
   i. SEQ ID NO:39 and 40,
   j. SEQ ID NO:41 and 42,
   k. SEQ ID NO:43 and 44,
   l. SEQ ID NO:51 and 52,
   m. SEQ ID NO:53 and 54,
   n. SEQ ID NO:55 and 56,
   o. SEQ ID NO:57 and 58,
   p. SEQ ID NO:59 and 60,
   q. SEQ ID NO:61 and 62 or
   r. SEQ ID NO:63 and 64, respectively.
104. The antibody or antigen-binding fragment thereof according to embodiment 57, wherein the antibody or antigen-binding fragment thereof belong to “Bin D”.
105. The antibody or antigen-binding fragment thereof according to embodiment 57, wherein the antibody or antigen-binding fragment thereof competes with a reference antibody wherein the reference antibody comprises a heavy chain variable domain identified by SEQ ID NO:47 and a light chain variable domain identified by SEQ ID NO:48.
106. The antibody according to the previous embodiment, wherein the reference antibody is a Fab.
107. The antibody or antigen-binding fragment thereof according to any of the previous embodiments, wherein the antibody or antigen-binding fragment thereof comprises
   a. a heavy chain variable domain at least 90% identical to the sequence identified by SEQ ID NO:47 and a light chain variable domain at least 90% identical to the sequence identified by SEQ ID NO:48,
   b. a heavy chain variable domain at least 90% identical to the sequence identified by SEQ ID NO:45 and a light chain variable domain at least 90% identical to the sequence identified by SEQ ID NO:46,
   c. a heavy chain variable domain at least 90% identical to the sequence identified by SEQ ID NO:51 and a light chain variable domain at least 90% identical to the sequence identified by SEQ ID NO:52 or
   d. a heavy chain variable domain at least 90% identical to the sequence identified by SEQ ID NO:61 and a light chain variable domain at least 90% identical to the sequence identified by SEQ ID NO:62.
108. The antibody or antigen-binding fragment thereof according to embodiment 107, wherein in the heavy chain variable domain is at least 92, 94, 96 or 98% identical to the identified SEQ IDs.
109. The antibody or antigen-binding fragment thereof according to embodiment 107, wherein in the light chain variable domain is at least 92, 94, 96 or 98% identical to the identified SEQ IDs.
110. The antibody or antigen-binding fragment thereof according to embodiment 108 and 109, wherein both the heavy chain variable domain and the light chain variable domain are at least 92, 94, 96 or 98% identical to the identified SEQ IDs.
111. The antibody or antigen-binding fragment thereof according to any of the previous embodiments, wherein the antibody or antigen-binding fragment thereof comprises
   a.
      i. three heavy chain CDR sequences with at most 10 amino acid changes compared to the CDR sequences of the heavy chain variable domain identified by SEQ ID NO:47 and
      ii. three light chain CDR sequences with at most 10 amino acid changes compared to the CDR sequences of the light chain variable domain identified by SEQ ID NO:48,
   b.
      i. three heavy chain CDR sequences with at most 10 amino acid changes compared to the CDR sequences of the heavy chain variable domain identified by SEQ ID NO:45 and
      ii. three light chain CDR sequences with at most 10 amino acid changes compared to the CDR sequences of the light chain variable domain identified by SEQ ID NO:46,
   c.
      i. three heavy chain CDR sequences with at most 10 amino acid changes compared to the CDR sequences of the heavy chain variable domain identified by SEQ ID NO:51 and
      ii. three light chain CDR sequences with at most 10 amino acid changes compared to the CDR sequences of the light chain variable domain identified by SEQ ID NO:52 or
   d.
      i. three heavy chain CDR sequences with at most 10 amino acid changes compared to the CDR sequences of the heavy chain variable domain identified by SEQ ID NO:61 and
      ii. three light chain CDR sequences with at most 10 amino acid changes compared to the CDR sequences of the light chain variable domain identified by SEQ ID NO:62.
112. The antibody or antigen-binding fragment thereof according to embodiment 111, wherein the three heavy chain CDR sequences have at most 9, such as 8, such as 7 or such as 6 amino acid changes compared to the CDRs of the identified SEQ IDs.
113. The antibody or antigen-binding fragment thereof according to embodiment 111, wherein the three heavy chain CDR sequences have at most 5, such as 4, such as 3, such as 2 or at most 1 amino acid changes compared to the CDRs of the identified SEQ IDs.
114. The antibody or antigen-binding fragment thereof according to embodiment 111, wherein the three light chain CDR sequences have at most 9, such as 8, such as 7 or such as 6 amino acid changes compared to the CDRs of the identified SEQ IDs.
115. The antibody or antigen-binding fragment thereof according to embodiment 111, wherein the three light chain CDR sequences have at most 5, such as 4, such as 3, such as 2 or at most 1 amino acid changes compared to the CDRs of the identified SEQ IDs.
116. The antibody or antigen-binding fragment thereof according to any of the previous embodiments, wherein the antibody or antigen-binding fragment thereof comprises
   a. the CDR sequences of the heavy chain variable domain identified by SEQ ID NO:47 and the CDR sequences of the light chain variable domain identified by SEQ ID NO:48,
   b. the CDR sequences of the heavy chain variable domain identified by SEQ ID NO:45 and the CDR sequences of the light chain variable domain identified by SEQ ID NO:46,
   c. the CDR sequences of the heavy chain variable domain identified by SEQ ID NO:51 and the CDR sequences of the light chain variable domain identified by SEQ ID NO:52
      or
   d. the CDR sequences of the heavy chain variable domain identified by SEQ ID NO:61 and the CDR sequences of the light chain variable domain identified by SEQ ID NO:62.
117. The antibody or antigen-binding fragment thereof according to any of the previous embodiments, wherein the antibody or antigen-binding fragment thereof comprises
   a. a heavy chain variable domain identified by SEQ ID NO:47 and a light chain variable domain identified by SEQ ID NO:48,
   b. a heavy chain variable domain identified by SEQ ID NO:45 and a light chain variable domain identified by SEQ ID NO:46,
   c. a heavy chain variable domain identified by SEQ ID NO:51 and a light chain variable domain identified by SEQ ID NO:52 or
   d. a heavy chain variable domain identified by SEQ ID NO:61 and a light chain variable domain identified by SEQ ID NO:62.
118. The antibody or antigen-binding fragment thereof according to embodiment 57, wherein the antibody or antigen-binding fragment thereof belongs “Bin E”.
119. The antibody or antigen-binding fragment thereof according to embodiment 57, wherein the antibody or antigen-binding fragment thereof competes with a reference antibody wherein the reference antibody comprises a heavy chain variable domain identified by SEQ ID NO:49 and a light chain variable domain identified by SEQ ID NO:50.
120. The antibody according to the previous embodiment, wherein the reference antibody is a Fab.
121. The antibody or antigen-binding fragment thereof according to any of the previous embodiments, wherein the antibody or antigen-binding fragment thereof comprises a heavy chain variable domain at least 90% identical to the sequence identified by SEQ ID NO:49 and a light chain variable domain at least 90% identical to the sequence identified by SEQ ID NO:50.
122. The antibody or antigen-binding fragment thereof according to embodiment 121, wherein in the heavy chain variable domain is at least 92, 94, 96 or 98% identical to the identified SEQ ID.
123. The antibody or antigen-binding fragment thereof according to embodiment 121, wherein in the light chain variable domain is at least 92, 94, 96 or 98% identical to the identified SEQ ID.
124. The antibody or antigen-binding fragment thereof according to embodiment 122 and 123, wherein both the heavy chain variable domain and the light chain variable domain are at least 92, 94, 96 or 98% identical to the identified SEQ IDs.
125. The antibody or antigen-binding fragment thereof according to any of the previous embodiments, wherein the antibody or antigen-binding fragment thereof comprises
   a. three heavy chain CDR sequences with at most 10 amino acid changes compared to the CDR sequences of the heavy chain variable domain identified by SEQ ID NO:49 and
   b. three light chain CDR sequences with at most 10 amino acid changes compared to the CDR sequences of the light chain variable domain identified by SEQ ID NO:50.
126. The antibody or antigen-binding fragment thereof according to embodiment 125, wherein the three heavy chain CDR sequences have at most 9, such as 8, such as 7 or such as 6 amino acid changes compared to the CDRs of the identified SEQ IDs.
127. The antibody or antigen-binding fragment thereof according to embodiment 125, wherein the three heavy chain CDR sequences have at most 5, such as 4, such as 3, such as 2 or at most 1 amino acid changes compared to the CDRs of the identified SEQ IDs.
128. The antibody or antigen-binding fragment thereof according to embodiment 125, wherein the three light chain CDR sequences have at most 9, such as 8, such as 7 or such as 6 amino acid changes compared to the CDRs of the identified SEQ IDs.
129. The antibody or antigen-binding fragment thereof according to embodiment 125, wherein the three light chain CDR sequences have at most 5, such as 4, such as 3, such as 2 or at most 1 amino acid changes compared to the CDRs of the identified SEQ IDs.
130. The antibody or antigen-binding fragment thereof according to any of the previous embodiments, wherein the antibody or antigen-binding fragment thereof comprises the CDR sequences of the heavy chain variable domain identified by SEQ ID NO:49 and the CDR sequences of the light chain variable domain identified by SEQ ID NO:50.
131. The antibody or antigen-binding fragment thereof according to any of the previous embodiments, wherein the antibody or antigen-binding fragment thereof comprises a heavy chain variable domain identified by SEQ ID NO:49 and a light chain variable domain identified by SEQ ID NO:50.
132. A multispecific antibody or antigen-binding fragment thereof capable of binding to FIX/FIXa and FX/FXa.
133. The multispecific antibody or antigen-binding fragment thereof according to embodiment 132, wherein the antibody comprises an antigen-binding fragment according to any of the previous embodiments 1-131.
134. The multispecific antibody or antigen-binding fragment thereof according to embodiment 132, wherein the antibody comprises an antigen-binding fragment according to any of the previous embodiments 2-30.
135. The multispecific antibody or antigen-binding fragment thereof according to embodiment 132, wherein the antibody comprises an antigen-binding fragment according to any of the previous embodiment 31-52.
136. The multispecific antibody or antigen-binding fragment thereof according to embodiment 132, wherein the antibody comprises an antigen-binding fragment according to any of the previous embodiments 57-131.
137. The multispecific antibody or antigen-binding fragment thereof according to embodiment 132, wherein the antibody comprises an antigen-binding fragment according to any of the previous embodiments 58-75.
138. The multispecific antibody or antigen-binding fragment thereof according to embodiment 132, wherein the antibody comprises an antigen-binding fragment according to any of the previous embodiments 76-89.
139. The multispecific antibody or antigen-binding fragment thereof according to embodiment 132, wherein the antibody comprises an antigen-binding fragment according to any of the previous embodiments 90-103.
140. The multispecific antibody or antigen-binding fragment thereof according to embodiment 132, wherein the antibody comprises an antigen-binding fragment according to any of the previous embodiments 104-117.
141. The multispecific antibody or antigen-binding fragment thereof according to embodiment 132, wherein the antibody comprises an antigen-binding fragment according to any of the previous embodiments 118-131
142. The multispecific antibody or antigen-binding fragment thereof according to embodiment 132, wherein the antibody comprises an antigen-binding fragment according to any of the previous embodiments 1-56 and an antigen-binding fragment according to any of the previous embodiments 57-131.
143. The multispecific antibody or antigen-binding fragment thereof according to embodiment 132, wherein the antibody comprises an antigen-binding fragment according to any of the previous embodiments 2-30 and an antigen-binding fragment according to any of the previous embodiments 58-75.
144. The multispecific antibody or antigen-binding fragment thereof according to embodiment 132, wherein the antibody comprises an antigen-binding fragment according to any of the previous embodiments 2-30 and an antigen-binding fragment according to any of the previous embodiments 76-89.
145. The multispecific antibody or antigen-binding fragment thereof according to embodiment 132, wherein the antibody comprises an antigen-binding fragment according to any of the previous embodiments 2-30 and an antigen-binding fragment according to any of the previous embodiments 90-103.
146. The multispecific antibody or antigen-binding fragment thereof according to embodiment 132, wherein the antibody comprises an antigen-binding fragment according to any of the previous embodiments 31-52 an antigen-binding fragment according to any of the previous embodiments 58-75.
147. The multispecific antibody or antigen-binding fragment thereof according to embodiment 132, wherein the antibody comprises an antigen-binding fragment according to any of the previous embodiments 31-52 and an antigen-binding fragment according to any of the previous embodiments 76-89.
148. The multispecific antibody or antigen-binding fragment thereof according to embodiment 132, wherein the antibody comprises an antigen-binding fragment according to any of the previous embodiments 31-52 and an antigen-binding fragment according to any of the previous embodiments 90-103.
149. The multispecific antibody or antigen-binding fragment thereof according to embodiment 132 wherein the antibody is a bispecific antibody capable of specifically binding FIX/FIXa and FX/FXa wherein the binding domains are derived the mAb pairs consisting of: mAb1-1371/mAb1-1307, mAb1-6705/mAb1-1307, mAb1-1371/mAb0-1886, mAb1-7441/mAb0-1886, mAb1-7447/mAb0-1886, mAb1-7481/mAb0-1886, mAb1-1371/mAb0-1998, mAb1-6716/mAb0-1998, mAb1-6723/mAb0-1998, mAb1-6730/mAb0-1998, mAb1-6731/mAb0-1998, mAb1-6737/mAb0-1998, mAb1-6754/mAb0-1998, mAb1-7378/mAb0-1998, mAb1-7441/mAb0-1998, mAb1-7447/mAb0-1998, mAb/mAb0-1998, mAb1-6723/mAb1-1307, mAb1-6723/mAb0-1886, mAb1-6705/mAb0-1886, mAb1-7481/mAb0-1998 and mAb1-6705/mAb0-1998.
150. The antibody or antigen-binding fragment thereof according to any one of embodiments 132 to 149 wherein the antibody is a procoagulant bispecific antibody.
151. The antibody or antigen-binding fragment thereof according to any one of embodiments 132 to 149 wherein the antibody is a bispecific antibody capable of increasing the procoagulant activity of FIXa.
152. The antibody or antigen-binding fragment thereof according to any of embodiments 132 to 149 wherein the antibody is a bispecific antibody capable of increasing the enzymatic activity of FIXa towards FX.
153. The antibody or antigen-binding fragment thereof according to any of embodiments 132 to 149 wherein the antibody is a bispecific antibody capable of functionally substituting for FVIII and/or FVIIIa.
154. A multispecific antibody capable of stimulating the enzymatic activity of FIXa towards FX comprising a first antigen-binding site recognizing FIX (SEQ ID NO:1) or the activated form thereof (FIXa), and a second antigen-binding site recognizing FX (SEQ ID NO:2) or the activated form thereof (FXa) wherein
   a) the first antigen-binding site comprises the CDRs of an antibody selected from the group consisting of: mAb1-5743, mAb1-6584, mAb1-8768, mAb1-6037, mAb1-6081, mAb1-4857, mAb1-8780, mAb1-9016, mAb1-9015, mAb1-8467, mAb1-5783, or mAb1-5781, and
   b) the second antigen-binding site comprises the CDRs of an antibody selected from the group consisting of: mAb1-6738, mAb1-6463, mAb1-6723, mAb1-7503, or mAb1-6097.
155. A multispecific antibody capable of stimulating the enzymatic activity of FIXa towards FX comprising
   a first polypeptide recognizing FIX/FIXa, and
   a second polypeptide recognizing FX/FXa wherein
   a) the first polypeptide comprises the heavy chain variable domain and light chain variable domain of mAb1-5743, mAb1-6584, mAb1-8768, mAb1-6037, mAb1-6081, mAb1-4857, mAb1-8780, mAb1-9016, mAb1-9015, mAb1-8467, mAb1-5783, or mAb1-5781, and
   b) the second polypeptide comprises the heavy chain variable domain and light chain variable domain of mAb1-6738, mAb1-6463, mAb1-6723, mAb1-7503, or mAb1-6097.
156. The antibody according to any one of embodiments 154 or 155 wherein the antibody is a bispecific antibody.
157. The antibody according to any one of embodiments 150-156 wherein the stimulation of the enzymatic activity of FIXa towards FX is determined in a FXa generation assay as described herein using a monovalent one-armed anti-FIX/FIXa antibody where in the stimulation index is at least 94, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 3000, 4000 or 5000-fold when measured using an one-armed antibody concentration resulting in at least 80% saturation of FIXa.
158. A multispecific antibody capable of stimulating the enzymatic activity of FIXa towards FX comprising
   a first antigen-binding site recognizing FIX (SEQ ID NO:1) or the activated form thereof (FIXa), and a second antigen-binding site recognizing FX (SEQ ID NO:2) or the activated form thereof (FXa), wherein
   the first antigen-binding site is capable of specifically binding an epitope comprising amino acid residue R338 of FIX/FIXa, and
   wherein the second antigen-binding site is capable of binding in the EGF-2 domain and/or catalytic subunit of FX/FXa.
159. The multispecific antibody according to embodiment 158 wherein the first antigen-binding site is capable of specifically binding an epitope comprising amino acid residues R338 and K341 of FIX/FIXa.
160. The multispecific antibody according to embodiment 159 wherein the first antigen-binding site is capable of specifically binding an epitope comprising amino acid residues L337, R338, S339, T340, K341 and T343 of FIX/FIXa.
161. A multispecific antibody capable of stimulating the enzymatic activity of FIXa towards FX comprising a first antigen-binding site recognizing FIX (SEQ ID NO:1) or the activated form thereof FIXa, and a second antigen-binding site recognizing FX (SEQ ID NO:2) or the activated form thereof (FXa)
   wherein
   the first antigen-binding site is capable of specifically binding an epitope comprising amino acid residues D332, R333, L337 and R338 of FIX/FIXa, and
   wherein the second antigen-binding site is capable of binding in the EGF-2 domain and/or catalytic subunit of FX/FXa.
162. The multispecific antibody according to embodiment 161 wherein the first antigen-binding site is capable of specifically binding an epitope comprising amino acid residues K301, D332, R333, A334, T335, R338, N346 of FIX/FIXa.
163. A multispecific antibody capable of stimulating the enzymatic activity of FIXa towards FX comprising
   a first antigen-binding site recognizing FIX/FIXa, and
   a second antigen-binding site recognizing FX/FXa
   wherein
   the first antigen-binding site is capable of specifically binding an epitope comprising amino acid residues H257, K293 and N406 of FIX/FIXa, and
   wherein the second antigen-binding site is capable of binding in the EGF-2 domain and/or catalytic subunit of FX/FXa.
164. The antibody according to any one of embodiments 1-56 and 132-156 wherein the stimulation of the enzymatic activity of FIXa towards FX is measured in a FXa generation assay as described herein using a monovalent one-armed anti-FIX/FIXa antibody wherein the stimulation index is at least 94, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 3000, 4000 or 5000 fold when measured using an one-armed antibody concentration resulting in at least 80% saturation of FIXa.
165. The antibody according to any one of embodiments 1-56 and 132-156 wherein the stimulation of the enzymatic activity of FIXa towards FX is measured in a FXa generation assay as described herein using a monovalent one-armed anti-FIX/FIXa antibody wherein the stimulation index is between 50 and 5000 fold, such as 94 to 2500 fold, such as 100 to 2500 fold, such as 200 to 2500 fold, such as 300 to 2500 fold, such as 400 to 2500 fold, such as 500 to 2500 fold, such as 600 to 2500 fold, such as 700 to 2500 fold, such as 800 to 2500 fold, such as 500 to 2500 fold, such as 600 to 2500 fold, such as 700 to 2500 fold, such as 800 to 2500 fold, such as 900 to 2500 fold, such as 1000 to 2500 fold or such as 1500 to 2500 fold when measured using an one-armed antibody concentration resulting in at least 80% saturation of FIXa.
166. The antibody according to any one of the previous embodiments wherein the antibody is a procoagulant antibody.
167. The antibody according to any one of the previous embodiments wherein said antibody functionally substitutes for FVIII and/or FVIIIa.
168. The antibody according to any one of the previous embodiments wherein the antibody is a bispecific antibody.
169. The antibody according to any one of the previous embodiments wherein the antibody isotype is IgG1, IgG2, IgG3 or IgG4 or a combination thereof.
170. An antibody according to any one of embodiments 57-131 wherein the light chain variable domain of said antibody or antigen-binding fragment thereof comprises amino acid residues R57, R96 (SEQ ID NO:22) and wherein the heavy chain variable domain of said antibody comprises amino acid residues W33, D52, D55, H100, Y101, Y102, H103 (SEQ ID NO:21).
171. The antibody or antigen-binding fragment thereof according to any of the previous embodiments for use in a method of treatment of a coagulopathy or blood coagulation disorder
172. A pharmaceutical composition comprising an antibody or antigen-binding fragment thereof according to any of the previous embodiments for the treatment of a coagulopathy or blood coagulation disorder.
173. A method of treating a subject suffering from a coagulopathy blood coagulation disorder, comprising administering to said subject an antibody or antigen-binding fragment thereof according to any of the previous embodiments.
174. A method according to embodiment 173 wherein the coagulopathy or blood coagulation disorder is haemophilia A or haemophilia A with inhibitors.

### EXAMPLES

### List of Abbreviations

ACN: Acetonitrile
CDR: Complementarity Determining Region
EGR-CK: EGR-chloromethylketone
LC-MS Liquid chromatography-mass spectrometry
FACS: Fluorescence-activated cell sorting
FIX: Coagulation Factor IX
FIXa: Coagulation Factor IXa
FX: Coagulation Factor X
FXa: Coagulation Factor Xa
HA: Haemophilia A
HA-PPP: HA-induced human platelet-poor plasma
HA-PRP: HA-induced human platelet-rich plasma
hFIXa: human Coagulation Factor IXa
ITC: Isothermal Titration Calorimetry
MACS: Magnetic-activated cell sorting
OA: One-armed
PCR: Polymerase Chain Reaction
SPR: Surface Plasmon Resonance

### Example 1: Development of Factor IX/FIXa Fab and mAb Expression Plasmids

FIX/FIXa binding antibodies as disclosed herein were identified using various antibody development methods. In order to generate a diverse set of antibodies targeting FIXa and FX, immunisations of mice and rabbits as well as selections from phage display and Adimab yeast display were performed.

### Adimab Yeast Display

The Adimab platform is a yeast display system encompassing a fully human naïve IgG1/kappa library with a diversity of 10¹⁰ and covering 20 out of 42 VH families. The utilized antibody phage display platform is a proprietary fully human Fab display library. The library has a size of 10¹⁰ and was constructed by a combinational approach utilizing chemical synthesis of the light chain, as well as the heavy chain CDR1 and CDR2, complemented with PCR amplification of the heavy chain CDR3 from human peripheral blood mononuclear cells.

The antibody selection process is directed using MACS and FACS based methods which allow monitoring of applied selection criteria in real time. Since selections are based on MACS and FACS, labelled antigens (e.g. biotin) are needed. Selection campaigns were performed using biotin-labelled active-site inhibited hFIXa (FIXa-EGR-biotin), or antibody mediated immobilization of hFIXa. Hits were evaluated for binding using Bio-layer interferometry (Octet fortebio systems).

### Phage Display

To maximise coverage of epitope diversity, different panning strategies were explored, including panning using biotinylated FIXa-EGR, FX, active-site inhibited FXa, or antigen capture using anti-FIXa antibodies. Initial hits were identified by phage ELISA. After sequence analysis, unique hits were cloned, expressed as IgG1, and ranked using SPR (Biacore) or Bio-layer interferometry (Octet fortebio systems).

### In Vivo Platforms

For generation of fully human antibodies in mice, Kymouse™ mice HK and HL 1.0 (utilizing kappa and lambda chains, respectively) were used. Additional immunisations were carried out in wild-type mice and rabbits in order to maximise antibody diversity.

### Generation of Anti-FIXa Antibodies

Mice or rabbits were immunized with FIXa, FIXa-EGR, or FX using standard protocols. Antibodies generated in mice or rabbits were screened in ELISA. FIXa binding rabbit B-cells were FACS-sorted using randomly biotinylated FIXa-EGR. Antibody hits from the rabbits and mice were either recombinantly expressed (rabbit mAbs) or propagated (mouse hybridomas) and antibodies were subsequently small scale purified.

### Generation of Anti-FX Antibodies

Kymouse mice and rabbits were immunised with FX using standard or Repetitive Immunization at Multiple Sites (RIMMS) protocols. Rabbit B-cells were isolated by FACS sorting using randomly biotinylated FX. Anti-FX mAbs from fusions and sortings were screened using ELISA and Octet fortebio systems.

### Sequencing of Kymouse and Wt Mouse Derived Antibodies

Anti-FIXa and anti-FX antibody producing hybridomas derived from Kymouse mice or wt mice were sequenced and expressed in HEK293 cells using standard techniques. Expressed antibodies were evaluated for binding using Octet fortebio systems.

Resulting variable domain (V_{H} and V_{L}) encoding DNA sequences of selected antibodies were inserted into a pTT-based mammalian expression vector (Durocher et al (2002) Nucleic Acid Res. 30: E9) or into a pcDNA3.4 mammalian expression vector (Invitrogen) containing antibody constant region encoding DNA sequences. For pTT/pcDNA3.4 mAb expression vectors, the V_{H} and V_{L} DNA sequences were inserted in-frame with human IgG1 or IgG₄ S228P (C_{H}1C_{H}2C_{H}3) or human C_{L} kappa constant region encoding DNA sequences, respectively. For the corresponding pTT/pcDNA3.4 Fab expression vectors the V_{H} DNA sequences were inserted in-frame with human IgG, C_{H}1 encoding DNA sequences.

For the 224F3 reference compound used in Example 6, 8 and 18 below, the 224F3 V_{H} and V_{L} sequences were obtained from EP1660536 B1 (SEQ ID NO:1 and 2, respectively). 224F3 V_{H} and V_{L} encoding DNA sequences were inserted into a pTT5/pcDNA3.4-based mammalian expression vector in-frame with human IgG1 (C_{H}1C_{H}2C_{H}3) or human C_{L} kappa constant region encoding DNA sequences, respectively.

All expression vectors included a 5′end DNA sequence containing a kozak sequence and a DNA sequence encoding a signal peptide in-frame with the antibody encoding DNA sequences.

### Example 2: Recombinant Expression of Antibodies and Antibody Fab Fragments

Antibodies and antibody Fab fragments were expressed using transient transfection of HEK293 suspension cells (293Expi, Invitrogen) essentially following manufacturer's instructions. 293Expi cells were typically subcultivated every 3-4 days in Expi293F expression medium (Invitrogen, catalogue number A1435104) supplemented with 1% P/S (GIBCO catalogue number 15140-122). Expi293F cells were transfected at a cell density of 2.5-3 mill/mL using Expifectamine. For each litre of Expi293F cells, the transfection was performed by diluting a total of 1 mg of plasmid DNA (V_{H}-C_{H}1 (for Fab) or V_{H}-C_{H}1-C_{H}2-C_{H}3 (for mAb) and LC plasmids in 1:1 ratio) into 50 mL Optimem (GIBCO, cat. no. 51985-026, dilution A) and by diluting 2.7 mL Expifectamine into 50 mL Optimem (dilution B). For Fab and mAb producing co-transfections, V_{H}-C_{H}1 and LC plasmids (Fab) and V_{H}-C_{H}1-C_{H}2-C_{H}3 and LC plasmids (mAb), respectively, were used in a 1:1 ratio. Dilution A and B were mixed and incubated at room temperature for 10-20 minutes. The transfection mix was hereafter added to the Expi293F cells and cells were incubated at 37° C. in a humidified incubator with orbital rotation (85-125 rpm). One day post-transfection, transfected cells were supplemented with 5 ml of ExpiFectamine 293 Transfection Enhancer 1 and 50 ml of ExpiFectamine 293 Transfection Enhancer 2. Cell culture supernatants were typically harvested 4-5 days post-transfection by centrifugation followed by filtration.

### Example 3: Fab and Antibody Purification and Characterization

### Fab Purification and Characterization

Purification of Fab molecules was conducted as a 2-step process composed of affinity chromatography using a kappaSelect resin (GE Healthcare, cat. no. 17-5458-11) and size-exclusion chromatography using a Superdex200 resin (GE Healthcare, cat. no. 17-1043-04). Purifications were conducted using an AktaExplorer chromatography system (GE Healthcare, cat. no. 18-1112-41). The buffer systems used for the affinity purification step were an equilibration buffer composed of 20 mM NaPhosphate pH 7.2, 150 mM NaCl and an elution buffer composed of 10 mM Formic acid pH 3.5 and a pH-adjustment buffer composed of 0.4 M NaPhosphate pH 9.0. Cell supernatants were applied directly without any adjustments onto a pre-equilibrated kappaSelect SuRe column. The column was washed with 10 column volumes of equilibration buffer and the Fab molecules were eluted isocratically in approx. 5 column volumes of elution buffer. The pH of the pooled fractions was adjusted to neutral using the described pH-adjustment buffer immediately after elution. The Fab molecules were further purified and buffer exchanged using said gel filtration resin pre-packed in a column. The running buffer used for size exclusion chromatography was 25 mM HEPES and 150 mM NaCl, pH 7.4. The Fab molecules eluted as single peaks at approx. 0.5 column volumes. Fractions covering the peak were analysed using a size-exclusion High-Performance Liquid Chromatographic (SE-HPLC) method setup on an Agilent LC 1100/1200 system and using a BIOSep-SEC-S3000 300×7.8 mm column (Phenomenex, cat. no. OOH-2146-K0) and a running buffer composed of 200 mM NaPhosphate pH 6.9, 300 mM NaCl and 10% isopropanol. Based on this analysis, fractions were pooled to obtain a homogenous protein preparation. The final preparation eluted as a single symmetric peak at a retention time of approx. 10 min at a flow rate of 1 ml/min.

The purified Fab molecules were further characterized using SDS-PAGE/Coomassie and liquid-chromatography mass spectrometry analyses. The SDS-PAGE/Coomassie analysis was performed using NuPage 4-12% Bis-Tris gels (Invitrogen, cat. no. NP0321 BOX). All Fab molecules displayed expected light chain and heavy chain components. Intact molecular mass determinations were performed using a Liquid Chromatography Electrospray Ionisation Time-of-Flight Mass Spectrometry method setup on an Agilent 6210 instrument and a desalting column MassPREP (Waters, cat. no. USRM10008656). The buffer system used was an equilibration buffer composed of 0.1% Formic acid in LC-MS graded-H₂O and an elution buffer composed of 0.1% formic acid in LC-MS graded-ACN. All Fab molecules displayed expected intact molecular masses in accordance with sequence. The final purity was determined based on SE-HPLC analysis. Purity estimates were all between 95-99% for the different Fab fragments. To determine the final protein concentrations, absorbance measurement at 280 nm using a NanoDrop spectrophotometer (Thermo Scientific) was performed and concentrations calculated using specific extinction coefficients for each of the Fab molecules.

### Antibody Purification and Characterization

Purification of the antibodies was conducted by affinity chromatography using a Protein A MabSelect SuRe resin (GE Healthcare, cat. no. 17-5438-01). Purifications were conducted using an AktaExplorer chromatography system (GE Healthcare, cat. no. 18-1112-41). The buffer systems used for the affinity purification step were an equilibration buffer was composed of 20 mM NaPhosphate pH 7.2, 150 mM NaCl and an elution buffer composed of 10 mM Formic acid pH 3.5 and a pH-adjustment buffer composed of 0.4 M NaPhosphate pH 9.0. Cell supernatants were applied directly without any adjustments onto a pre-equilibrated MabSelect SuRe column. The column was washed with 10 column volumes of equilibration buffer and the antibodies were eluted isocratically in approx. 2-5 column volume of elution buffer. The pH of the pooled fractions was adjusted to neutral using the described pH-adjustment buffer immediately after elution.

The purified antibodies were characterized using SDS-PAGE/Coomassie, size-exclusion high-pressure liquid-chromatography (SE-HPLC) and liquid-chromatography mass spectrometry (LC-MS) analyses. The SDS-PAGE/Coomassie analysis was performed using NuPage 4-12% Bis-Tris gels (Invitrogen, cat. no. NP0321 BOX). Here, all antibodies displayed expected light chain and heavy chain components. Intact molecular mass determinations were performed using a Liquid Chromatography Electrospray Ionisation Time-of-Flight Mass Spectrometry method setup on an Agilent 6210 instrument and a desalting column MassPREP (Waters, cat. no. USRM10008656). The buffer system used was an equilibration buffer composed of 0.1% Formic acid in LC-MS graded-H₂O and an elution buffer composed of 0.1% formic acid in LC-MS graded-ACN. Analyses were performed with and without N-Glycosidase F (Roche Diagnostics, cat. no. 11365177001) and reducing agent (i.e. mercaptoethanol or DTT). All antibodies displayed expected intact molecular masses in accordance with sequence and one heavy chain N-glycan. Purity was determined based on SE-HPLC. The final protein purity was analysed based on SE-HPLC method setup on an Agilent LC 1100/1200 system and using a BIOSep-SEC-S3000 300×7.8 mm column (Phenomenex, cat. no. OOH-2146-K0) and a running buffer composed of 200 mM NaPhosphate pH 6.9, 300 mM NaCl and 10% isopropanol. UV280 and fluorescence (Ex 280 nm/Em 354 nm) detectors was used for detection. The antibodies eluted as single symmetric peaks with retention times reflecting the size of the antibodies. Purity estimates were all between 95-99% for the different antibodies. To measure the final protein concentrations, a NanoDrop spectrophotometer (Thermo Scientific) was used together with specific extinction coefficients for each of the antibodies.

### Example 4: Binning of Anti-FIXa Stimulating Antibodies

Antibodies selected as capable of stimulating the enzymatic activity of FIXa towards FX were analysed in binning experiments to determine the binding characteristics for the identified antibodies using the method described below.

### Method for Binning of Antibodies

Binning experiments were performed using Octet fortebio systems (HTX, Red384) equipped with anti-human IgG sensors (Pall Life Sciences, Menlo Park, CA), and using 8 or 32-channel mode (Red384 and HTX). The binning assays were performed using the classical sandwich epitope binning setup. Briefly, (1) the first antibody was captured by anti-human AHC tips (anti-human IgG Fc capture tips (AHC Part NO:18-5064), (2) non-blocked IgG binding sites on the AHC tips were blocked by human polyclonal IgG (14506 SIGMA), (3) FIXa was bound to the first antibody, (4) the competing antibody was offered to the antibody-antigen complex on the tips, and if no binding of the secondary antibody could be detected, the antibodies were scored as belonging to the same bin.

The analysis identified two different bins, Bin 1 and 2, defined by the antibodies mAb0-1886 and mAb1-1307, respectively.

### Binning of Anti-FIX Antibodies.

Selected anti-FIX antibodies were binned against each other, and two different bins (Bin 1 and 2) were identified. Numbers refer to mAb ID, e.g. 0-1998 denotes mAb0-1998.

| Bin 1 | Bin 2 |
|---|---|
| mAb0-1886 | mAb1-1307 |
| 0-1998 | |
| 0-2000 | |
| 0-2001 | |
| 0-2003 | |
| 1-0985 | |
| 1-0982 | |
| 1-0072 | 1-0072 |
| 1-0073 | 1-0073 |
| | 1-1448 |
| | 1-0970 |

The overview shows that several antibodies were found to belong to Bin 1, including mAb0-1886 and mAb0-1998. Bin 2 was found to include four antibodies in addition to mAb1-1307. Two antibodies, mAb1-0072 and mAb1-0073, were common to Bin 1 and 2.

Variants of Parental Antibodies (Lineages) as Disclosed Herein Share Bins and Epitope (Hot-Spot) Residues with Parental Antibodies

Since the antibody variants for which data are provided in the present example do not contain amino acid substitutions on positions shown to be crucial for epitope recognition based on the crystal structures of the parental antibody-FIXa complexes provided in Example 5, a person skilled in the art would understand that the variants as a starting point will belong to the same bin, compete for binding with, and recognize at least the same hot-spot residues in the FIX/FIXa epitope as the antibody from which they originate, i.e. mAb0-1998, mAb0-1886, or mAb1-1307.

### Example 5: Crystallization and Epitope Mapping of Anti-FIX/FIXa Antibodies Using X-Ray Crystallography

The FIXa protein used for crystallization (Cambridge ProteinWorks, Product Code 10316) is composed of a truncated light chain (residues 85-142 of SEQ ID NO:1) with a non-natural methionine residue attached at the N-terminus as a result of bacterial expression, and a heavy chain containing residues 181-415 of SEQ ID NO:1. The active site of the protease is blocked by EGR-chloromethylketone.

### Crystallisation

### Fab0-7237:FIXa

Crystals of Fab0-7237 (Fab fragment corresponding to mAb0-1886) mixed in a 1:1 molar ratio with the FIXa protein were grown using the hanging drop vapour diffusion technique at 18° C. A protein solution of 0.8 μl 7.5 mg/ml in 20 mM Tris-HCl, pH 7.4, 50 mM NaCl, and 2.5 mM CaCl₂ was mixed with an equal volume of 4 M sodium formate as precipitant and incubated over 1 ml precipitant.

### Fab0-7238:FIXa

Crystals of Fab0-7238 (Fab fragment corresponding to mAb0-1998) mixed in a 1:1 molar ratio with the FIXa protein were grown using the sitting drop vapour diffusion technique at 18° C. A protein solution of 0.1 μl 6.2 mg/ml in 20 mM Tris-HCl, pH 7.4, 50 mM NaCl, and 2.5 mM CaCl₂ was mixed with 0.1 μl of 100 mM sodium cacodylate, pH 6.5 and 1 M tri-sodium citrate as precipitant and incubated over 60 μl precipitant.

### Fab0-7236:FIXa

Crystals of Fab0-7236 (Fab fragment corresponding to mAb1-1307) mixed in a 1:1 molar ratio with the FIXa protein were grown using the sitting drop vapour diffusion technique at 18° C. A protein solution of 0.1 μl 6.4 mg/ml in 20 mM Tris-HCl, pH 7.4, 50 mM NaCl, and 2.5 mM CaCl₂ was mixed with an equal volume of 0.2 M lithium sulphate, 40 (v/v) % PEG400, and 0.1 M Tris pH, 8.5 as precipitant and incubated over 1 ml precipitant.

### Diffraction Data Collection

### Fab0-7237:FIXa

The crystal was cryo protected in a solution consisting of 3 M sodium formate, 4% glycerol, 4% ethylene glycol, 4.5% sucrose, and 1% glucose prior to flash cooling in liquid nitrogen. Diffraction data were collected at 100K at the Swiss Light Source beamline X06DA (1.0000 Å wavelength) using a Pilatus2M pixel detector from Dectris. Autoindexing, integration and scaling of the data were performed with programmes from the XDS package (diffracting data statistics are summarised in Table 1).

### Fab0-7238:FIXa

The crystal was cryo protected in a solution consisting of 75 mM sodium cacodylate, pH 6.5 and 0.75 M tri-sodium citrate, 4% glycerol, 4% ethylene glycol, 4.5% sucrose, and 1% glucose prior to flash cooling in liquid nitrogen. Diffraction data were collected at 100K at the Swiss Light Source beamline X06DA (1.0000 Å wavelength) using a Pilatus2M pixel detector from Dectris. Autoindexing, integration and scaling of the data were performed with programmes from the XDS package (diffracting data statistics are summarised in Table 1).

### Fab0-7236:FIXa

Three crystals were cryo protected in a solution consisting of 0.15 M lithium sulphate, 30 (v/v) % PEG400, and 0.075 M Tris pH, 8.5, 4% glycerol, 4% ethylene glycol, 4.5% sucrose, and 1% glucose prior to flash cooling in liquid nitrogen. Diffraction data were collected at 100K at the Swiss Light Source beamline X06DA (1.0000 Å wavelength) using a Pilatus2M pixel detector from Dectris. Autoindexing, integration, merging and scaling of the data were performed with programmes from the XDS package (diffracting data statistics are summarised in Table 1).

### Structure Determination and Refinement

### Fab0-7237:FIXa

The structure was determined by molecular replacement using Phaser as implemented in the programme suite Phenix with the chains H and L of protein data bank entry 4NP4 and chains H and L from protein data bank entry 3KCG. The asymmetric unit contains two Fab:FIXa complexes. The model was refined using steps of Phenix refinement and manual rebuilding in COOT. The refinement statistics are found in Table 1.

### Fab0-7238:FIXa

The structure was determined by molecular replacement using Phaser as implemented in the programme suite Phenix with the chains H and L of protein data bank entry 4PUB and chains H and L from protein data bank entry 3KCG. The asymmetric unit contains two Fab:FIXa complexes. The model was refined using steps of Phenix refinement and manual rebuilding in COOT. The refinement statistics are found in Table 1.

### Fab0-7236:FIXa

The structure was determined by molecular replacement using Phaser as implemented in the programme suite Phenix with the Fab part of complex structure of Fab0-7238:FIXa complex described above and chains H and L from protein data bank entry 3KCG. The asymmetric unit contains one Fab:FIXa complex. The model was refined using steps of Phenix refinement and manual rebuilding in COOT. The refinement statistics are found in Table 1.

TABLE 1
Data collection and refinement statistics

| | Fab0-7237 in | | Fab0-7238 in | | Fab0-7236 in | |
|---|---|---|---|---|---|---|
| | complex with | | complex with | | complex with | |
| | Gladomaineless | | Gladomaineless | | Gladomaineless | |
| | FIXa (WT) | | FIXa (WT) | | FIXa (WT) | |
| | EGR-CK inhibited | | EGR-CK inhibited | | EGR-CK inhibited | |
| Wavelength (Å) | 1.0000 | | 1.0000 | | 1.0000 | |
| Resolution range (Å) | 48.36-2.45 | (2.538-2.45) | 45.71-2.05 | (2.123-2.05) | 43.64-2.0 | (2.071-2.0) |
| Space group | P6₁ | | C2 | | I222 | |
| Unit cell (Å, °) | 243.41 243.41 72.29 90 90 120 | | 369.38 94.42 60.93 90 98.868 90 | | 87.18 96.86 201.38 90 90 90 | |
| Total reflections | 927423 | (88310) | 443072 | (43104) | 1167939 | (113684) |
| Number of crystals | 1 | | 1 | | 3 | |
| Unique reflections | 90260 | (8982) | 128149 | (12523) | 57871 | (5716) |
| Multiplicity | 10.3 | (9.8) | 3.5 | (3.4) | 20.2 | (19.9) |
| Completeness (%) | 99.96 | (99.94) | 98.73 | (97.31) | 99.89 | (99.79) |
| Mean I/sigma(I) | 9.98 | (1.10) | 13.50 | (1.12) | 9.06 | (1.26) |
| Wilson B-factor | | | 39.05 | | 30.10 | |
| R-merge | 0.2704 | (2.638) | 0.08249 | (1.269) | 0.3714 | (3.07) |
| R-meas | 0.2847 | (2.784) | 0.09768 | (1.503) | 0.381 | (3.151) |
| R-pim | 0.08848 | (0.8856) | 0.05184 | (0.7982) | 0.08447 | (0.7044) |
| CC½ | 0.991 | (0.348) | 0.998 | (0.571) | 0.995 | (0.595) |
| CC* | 0.998 | (0.718) | 1 | (0.853) | 0.999 | (0.864) |
| Reflections used in refinement | 90261 | (8984) | 128040 | (12521) | 57825 | (5715) |
| Reflections used for R-free | 1928 | (199) | 1611 | (157) | 1461 | (143) |
| R-work | 0.2013 | (0.3016) | 0.2242 | (0.4200) | 0.1971 | (0.3096) |
| R-free | 0.2411 | (0.3533) | 0.2607 | (0.4969) | 0.2454 | (0.3350) |
| CC(work) | 0.904 | (0.507) | 0.949 | (0.654) | 0.964 | (0.755) |
| CC(free) | 0.884 | (0.269) | 0.954 | (0.435) | 0.938 | (0.663) |

| Number of non-hydrogen atoms | 11731 | 11849 | 6132 |
|---|---|---|---|
| macromolecules | 11219 | 11196 | 5579 |
| ligands | 52 | 2 | 61 |
| solvent | 460 | 651 | 492 |
| Protein residues | 1465 | 1456 | 720 |
| RMS(bonds) | 0.015 | 0.008 | 0.008 |
| RMS(angles) | 1.22 | 1.15 | 1.13 |
| Ramachandran favored (%) | 90.91 | 95.28 | 96.20 |
| Ramachandran allowed (%) | 7.70 | 4.65 | 3.38 |
| Ramachandran outliers (%) | 1.39 | 0.07 | 0.42 |
| Rotamer outliers (%) | 6.46 | 0.40 | 0.81 |
| Clashscore | 8.48 | 6.71 | 5.39 |
| Average B-factor | 46.41 | 49.46 | 38.01 |
| macromolecules | 46.59 | 49.64 | 37.38 |
| ligands | 56.40 | 79.63 | 69.03 |
| solvent | 41.00 | 46.15 | 41.27 |
| Number of TLS groups | 40 | | |
| Twin refinement | h, -h-k, -l | | |

Statistics for the highest-resolution shell are shown in parentheses.

### Determination of Epitopes

Based on the above mAb0-1998, mAb1-1307 and mAb0-1886 were found to bind to different epitopes on FIXa where the epitope is defined as residues having at least one heavy atom within a distance of ≤3.5 Å from a heavy atom in the antibody.

The mAb0-1998 epitope is located in the 170-loop and comprises the following residues in the protease domain: L337, R338, S339, T340, K341, and T343.

The mAb1-1307 epitope comprises the following residues: H256, H257, N258, K293, R403, Y404, N406, W407, E410 and K411.

The mAb0-1886 epitope is located in the 170-helix and comprises of the following residues in the protease domain: K301, D332, R333, A334, T335, R338 and N346.

The epitopes of mAb0-1998 and mAb0-1886 were found to be overlapping which corresponds well with the observation that the two antibodies compete for binding to FIX/FIXa (Example 4).

Variants of Parental Antibodies (Lineages) as Disclosed Herein Share Bins and Epitope (Hot-Spot) Residues with Parental Antibodies

Since the antibody variants for which data are provided in the Example 4 above and certain examples below do not contain amino acid substitutions on positions shown to be crucial for epitope recognition based on the crystal structures of the parental antibody-FIXa complexes provided in the present example, a person skilled in the art would understand that the variants as a starting point will belong to the same bin, compete for binding with, and recognize at least the same hot-spot residues in the FIX/FIXa epitope as the antibody from which they originate, i.e. mAb0-1998, mAb0-1886, or mAb1-1307.

### Example 6: Activity of Bivalent Anti-FIX/FIXa Antibodies in a FXa Generation Assay

The stimulatory effect on the enzymatic activity of FIXa towards FX of bivalent anti-FIX/FIXa antibodies was determined from their ability to promote FX activation by FIXa in the presence of a procoagulant phospholipid membrane according to the principles described by Scheiflinger et al. (2008) J Thromb Haemost, 6:315-322. Given the high activity of anti-FIXa antibody 224F3 among the antibodies identified by Scheiflinger et al. 224F3 was chosen as reference in the following experiments (cf. Example 1 for information on 224F3 construction). The stimulating effect of anti-FIXa antibodies on FIXa mediated activation of FX into FXa was measured in an automated high through-put biochemical assay in 384-well plates. In brief, FIXa was mixed with purified antibody in a four-point 5-fold dose-response. FX/phospholipid (PL) mix was added and FXa generation was measured by adding FXa substrate (Pefaflour) and the substrate hydrolysis rate determined by detecting fluorescence for five minutes on a multi-label reader (PheraSTAR). Relative FIXa stimulatory activity was calculated as the rate of FXa generation from FIXa-antibody complex versus FIXa alone.

Each antibody was tested in a concentration range from 0-200 nM by pre-incubation with 3 nM human plasma-derived FIXa (Haematologic Technologies Inc, USA) and 10 μM 25:75 phosphatidyl serine:phosphatidyl choline phospholipid vesicles (Haematologic Technologies Inc, USA) in assay buffer (50 mM HEPES, 100 mM NaCl, 5 mM CaCl₂, 0.1% (w/v) PEG8000, pH 7.3+1 mg/ml BSA) for 10 min before addition of human plasma-derived FX (Haematologic Technologies Inc, USA) to a concentration of 150 nM. Following 10 min activation at room temperature, the reaction (50 μl) was quenched by addition of 25 μl quench buffer (50 mM HEPES, 100 mM NaCl, 60 mM EDTA, 0.1% PEG8000, pH 7.3+1 mg/ml BSA). The amount of FXa generated was then determined by addition of 25 μl 2 mM S-2765 chromogenic substrate (Chromogenix, Sweden) and measurement of chromogenic substrate conversion by absorbance measurement at 405 nm (ΔOD/min) in a microplate reader. The rate of FXa generation at each antibody concentration was determined from a standard curve made with known amounts of human plasma-derived FXa (Haematologic Technologies Inc, USA).

Table 2 lists the measured FXa generation rates for each antibody at the concentrations tested. From this, peak stimulatory activities were calculated for each antibody as the observed maximum FXa generation rate relative to that of 224F3. This data is presented in Table 3, which shows that antibodies belonging to each of the three families (0-1886, 0-1998, and 1-1307, respectively) have activities that are 10-67 times higher than that observed for 224F3 (Scheiflinger et al.).

TABLE 2
FXa generation rates
FXa generation rates in pM/min (mean ± SD, n = 2) for the listed anti-FIX/FIXa antibodies.
Each antibody was tested in a concentration range from 0-200 nM as indicated in the first column.

| mAb conc. (nM) | 1-4857 | 1-4861 | 1-4707 | 1-4763 | 1-4071 | 1-4624 | 224F3 |
|---|---|---|---|---|---|---|---|
| 200 | 1300 ± 136 | 411 ± 39 | 586 ± 23 | 401 ± 25 | 208 ± 1 | 960 ± 10 | 5.5 ± 0.2 |
| 100 | 1262 ± 1 | 364 ± 18 | 581 ± 13 | 375 ± 1 | 198 ± 4 | 1001 ± 28 | 7.2 ± 0.5 |
| 50 | 1192 ± 37 | 352 ± 4 | 629 ± 8 | 369 ± 2 | 173 ± 2 | 989 ± 21 | 9.0 ± 0.2 |
| 25 | 1004 ± 42 | 382 ± 14 | 685 ± 20 | 373 ± 1 | 131 ± 4 | 851 ± 72 | 9.0 ± 3.6 |
| 12.5 | 685 ± 18 | 385 ± 7 | 697 ± 14 | 381 ± 10 | 97 ± 3 | 752 ± 1 | 14.7 ± 0.3 |
| 6.3 | 491 ± 6 | 415 ± 12 | 714 ± 34 | 390 ± 17 | 65 ± 4 | 550 ± 5 | 17.4 ± 0.1 |
| 3.2 | 302 ± 3 | 434 ± 15 | 662 ± 37 | 370 ± 15 | 43 ± 3 | 383 ± 3 | 20.0 ± 0.4 |

TABLE 3
Peak stimulatory activities
Peak stimulatory activities (mean ± SD, n = 2)
of anti-FIX/FIXa antibodies relative to 224F3
(Scheiflinger et al.) in the FXa generation assay.

| | | Peak stimulatory |
|---|---|---|
| | Antibody | activity relative |
| mAb ID | family | to 224F3 |
| 1-4857 | 0-1998 | 66.5 ± 6.1 |
| 1-4861 | 0-1998 | 21.7 ± 1.2 |
| 1-4707 | 1-1307 | 35.9 ± 2.1 |
| 1-4763 | 1-1307 | 20.0 ± 1.7 |
| 1-4071 | 0-1886 | 10.4 ± 0.2 |
| 1-4624 | 0-1886 | 50.1 ± 2.4 |
| 224F3 | | 1.0 |

### Example 7: Preparation of Monovalent (One-Armed) Antibodies

To avoid any potential avidity effects associated with conventional monospecific and bivalent antibodies, e.g. in FXa generation assays (Example 8) and in certain SPR experiments (example 14 and 15), a monovalent one-armed (OA) antibody format was used, as described by Martens et al.: A Novel One-Armed Anti-c-Met Antibody Inhibits Glioblastoma Growth In vivo. Clin. Cancer Res. 12, 6144-6152 (2006), where a full heavy chain, a truncated heavy chain (lacking the Fab region) and a light chain are co-expressed. Instead of co-expression of the three chains described by Martens et al. monovalent antibodies were in the present invention prepared using the Duobody® principle as described for bispecific antibodies (Example 10). Thus, monovalent antibodies were prepared by mixing a full monospecific and bivalent antibody and a truncated heavy chain dimer (formally derived from a full antibody by removing the Fab region) and allow exchange of chains to proceed under the same experimental conditions as described in Example 10. Formation of the monovalent antibody requires that the antibody and truncated heavy chain dimer carry appropriate complementary mutations to promote hetero-dimerization, i.e. F405L/K409R for IgG1 and F405L+R409K/WT for IgG4, as described in Example 10.

In case of monovalent antibodies of the IgG1 subtype the truncation of the heavy chain can be from the N-terminus to a position in-between Cys 220 and the upper hinge Cys 226 (EU numbering). A specific example of a truncated IgG1 heavy chain is one where residues 1-220 are truncated.

In case of monovalent antibodies of the IgG4 subtype the truncation of the heavy chain can be from the N-terminus to a position in-between Cys 200 and the upper hinge Cys 226 (EU numbering). A specific example of a truncated IgG4 heavy chain is one where residues 1-214 are truncated.

### Example 8: Activity of Monovalent Anti-FIX/FIXa Antibodies in a FXa Generation Assay

To avoid any potential avidity effects arising as a consequence of the bivalency of the conventional antibody format, the stimulatory activity of anti-FIX/FIXa antibodies on FIXa enzymatic activity towards FX was determined following reformatting into a monovalent one-armed (OA) antibody format (see Example 8). Tested antibodies are listed in Table 4 below. The monovalent OA version of the anti-FIXa antibody 224F3 (denoted mAb1-1582), also referred to in Example 7, was included for comparison.

The stimulatory activity of OA antibodies was measured in assay buffer (50 mM HEPES, 100 mM NaCl, 5 mM CaCl₂, 0.1% (w/v) PEG8000, pH 7.3+1 mg/ml BSA) at fixed concentrations of phosphatidyl serine (PS):phosphatidyl choline (PC) phospholipid vesicles (final concentration of 500 μM; Haematologic Technologies Inc, USA) and plasma-derived FIXa (final concentrations of 0.17, 0.5 or 1 nM; Haematologic Technologies Inc, USA). The concentration of FIXa was chosen to ensure that less than 15% of the substrate FX was converted into FXa. Following pre-incubation in the presence of monovalent OA antibody (final concentrations listed in Table 1), 150 nM plasma-derived FX was added to give a final reaction volume of 50 μl, and activation was allowed to proceed for 20 min at room temperature. The reaction was then quenched by addition of 25 μl quench buffer (50 mM HEPES, 100 mM NaCl, 60 mM EDTA, 0.1% PEG8000, pH 7.3+1 mg/ml BSA) and the amount of FXa generated was determined by further addition of 25 μl 2 mM S-2765 chromogenic substrate (Chromogenix, Sweden) and measurement of chromogenic substrate conversion by absorbance measurement at 405 nm (ΔOD/min) in a microplate reader. The measured activity was corrected for background activity by subtraction of the signal measured in the same assay but with FIXa and antibody replaced by assay buffer, and then normalized according to the concentration of FIXa present in the assay ([FIXa]ₜₒₜₐₗ). Dividing this number by the similarly normalized rate of FXa generation in the absence of antibody (A_{FIXa,norm}), an antibody stimulation index was calculated providing the fold stimulation of FIXa activity by the antibody at the concentration used. Due to slow rate of FXa generation by free FIXa, activation reactions in the absence of antibody were carried out as described above but with 5, 10, or 20 nM FIXa present. Measured activities were then background subtracted and normalized according to the FIXa concentration in the assay. For the calculation of the stimulation index, the average of the three normalized activities of free FIXa was used.

### Determination of Stimulation Index

In summary, calculation of the stimulation index can be described as follows

$$Stimulation index = ( ( {A}_{FIXa + OA} - {A}_{bckg} ) {/ [ FIXa ]}_{total} ) / {A}_{FIXa , norm}$$

where A_{FIXa+OA} is the activity measured in the presence of OA antibody, A_{bckg} is the background activity measured in the absence of FIXa and monovalent antibody, [FIXa]ₜₒₜₐₗ is the FIXa concentration in the assay, and A_{FIXa,norm} is average normalized activity of free FIXa.

### Determination of FIXa Saturation

The fraction of FIXa saturated with OA antibody in the assay is determined by the concentrations of FIXa and OA antibody, and the equilibrium dissociation constant (K_{d}) governing their interaction. The latter can be measured by techniques known in the art, such as isothermal titration calorimetry (ITC).

Since the stimulation index will increase as the concentration of OA antibody is increased until saturation of FIXa is reached, the concentration of OA antibody in the assay should be chosen to ensure at least 80% saturation of FIXa in the assay to provide a proper estimate of the stimulation index at full FIXa saturation.

The fraction of FIXa bound to OA antibody at equilibrium (f_{FIXa+OA}), can be calculated from the total concentrations of FIXa ([FIXa]ₜₒₜₐₗ) and OA ([OA]ₜₒₜₐₗ) in the assay and the equilibrium dissociation constant (K_{d}) for their interaction using the quadratic binding equation as described by Krishnaswamy et al. (1992) J. Biol. Chem., 267:23696-23706 and detailed in Eq. 1 and 2 below, wherein

[FIXa+OA]_{assay} represents the calculated concentration of FIXa-OA antibody complex at equilibrium in the assay
f_{FIXa+OA} represents the calculated fraction (in percent) of FIXa, which is bound to OA antibody at equilibrium in the assay

$${[ FIXa + OA ]}_{assay} = \qquad Eq . 1$$

$$\frac{\begin{matrix}( {[ FIXa ]}_{total} + {[ OA ]}_{total} + {K}_{d} ) - \\ \sqrt{{( {[ FIXa ]}_{total} + {[ OA ]}_{total} + {K}_{d} )}^{2} - 4 \times {[ FIXa ]}_{total} \times {[ OA ]}_{total}}\end{matrix}}{2}$$

$${f}_{FIXa + OA} = 100 \% \times \frac{{[ FIXa + OA ]}_{assay}}{{[ FIXa ]}_{total}} \qquad Eq . 2$$

The stimulation index for each monovalent OA antibody is provided in Table 4. For all tested antibodies the measured stimulation index was found to be higher than that measured for the monovalent one-armed 224F3 antibody (mAb1-1582).

With a concentration of one-armed 224F3 antibody of 3260 nM in the assay and a K_{d} for the interaction with FIXa of 0.477 nM as reported by Kerschbaumer et al. (U.S. Pat. No. 7,297,336-B2), more than 95% of FIXa was bound to the one-armed 224F3 antibody in the assay.

TABLE 4
Stimulation of FIXa activity by monovalent one-armed
(OA) anti-FIXa antibodies
The anti-FIX mAb ID refers to the ID of the antibody used
for reformatting into the OA format. Columns labelled
‘OA antibody concentration (nM)’ and ‘Stimulation
index’ list the concentration of OA antibody (nM) used
in the assay and the corresponding stimulation of FIXa
activity measured relative to free FIXa.

| anti-FIX | | OA antibody | Stimulation |
|---|---|---|---|
| mAb ID | Lineage | concentration (nM) | Index |
| 1-7977 | 0-1998 | 800 | 850 |
| 1-8785 | 0-1998 | 800 | 621 |
| 1-8782 | 0-1998 | 1600 | 417 |
| 1-8780 | 0-1998 | 1600 | 1651 |
| 1-8543 | 0-1998 | 1600 | 283 |
| 1-8679 | 0-1998 | 1600 | 918 |
| 1-9016 | 0-1998 | 1600 | 4319 |
| 1-9015 | 0-1998 | 1600 | 1334 |
| 1-9002 | 0-1998 | 800 | 567 |
| 1-9058 | 0-1998 | 1600 | 531 |
| 1-9134 | 0-1998 | 1600 | 320 |
| 1-8467 | 0-1998 | 1600 | 1020 |
| 1-9285 | 0-1998 | 800 | 732 |
| 1-8459 | 0-1998 | 1600 | 799 |
| 1-5797 | 0-1886 | 474 | 382 |
| 1-5796 | 0-1886 | 900 | 239 |
| 1-5783 | 0-1886 | 264 | 1036 |
| 1-5781 | 0-1886 | 564 | 1219 |
| 1-5754 | 0-1886 | 1478 | 615 |
| 1-6609 | 0-1886 | 800 | 94 |
| 1-6606 | 0-1886 | 800 | 133 |
| 1-6592 | 0-1886 | 800 | 916 |
| 1-6590 | 0-1886 | 800 | 1287 |
| 1-6586 | 0-1886 | 800 | 94 |
| 1-6584 | 0-1886 | 800 | 580 |
| 1-6582 | 0-1886 | 800 | 692 |
| 1-6566 | 0-1886 | 204 | 690 |
| 224F3 | — | 3260 | <10 |
| (1-1582) | | | |

### Example 9: Development of Anti FX/FXa Fab and mAb Expression Plasmids

Anti-FX/FXa antibodies as disclosed herein were developed using standard antibody development methods and expression plasmids were prepared as described in Example 1 for the anti-FIX/FIXa Fab and mAb expression plasmids. Expression, purification and characterisation of anti-FX/Xa antibodies were likewise performed as described for anti-FIX/FIXa antibodies in Examples 2 and 3.

### Example 10: Bispecific Antibodies Prepared by In Vitro Assembly

Bispecific antibodies are generated by in vitro assembly of a first and a second antibody by the Duobody® method (Genmab) described (Labrijn et al. PNAS 2013, vol. 110, pp. 5145-5150) for bispecific IgG1 antibodies and using a slightly modified variant for bispecific IgG4 antibodies as detailed in the following.

For IgG1 the heavy chain constant region of the first antibody is IgG1 K409R (anti-FIX/FIXa) and the heavy chain constant region of the second antibody is IgG1 F405L (anti-FX/FXa). The IgG1 may be a IgG1 variant with reduced effector functions, as referred to earlier.

For IgG4 the heavy chain constant region of the first antibody is IgG4 S228P (anti-FIX/FIXa) and the heavy chain constant region of the second antibody is IgG4 S228P F405L R409K (anti-FX). The two parental antibodies are produced as described in Examples 1-3. The Fab arm exchange reaction is carried out in HEPES buffer (pH 7.4) under reducing conditions using 75 mM 2-mercaptoethylamine (2-MEA) and incubation at 30° C. for 3 hours.

### Example 11: Procoagulant Activities of Bispecific Antibodies

Pairs of anti-FIXa and anti-hFX antibodies were made into bispecific antibodies by means of the Duobody® technology as described above (Example 10). The bispecific antibodies were tested for procoagulant activity in various assays, such as the FXa generation assays described above (Example 6) and in a Thrombin-Generation Test (TGT) as described in the following paragraph.

### Thrombin Generation Test (TGT) Assay

TGT was conducted in an automated HTP 384-well setup using kaolin triggering (Haemonetics Corporation, #6300). In brief, antibodies were added at a concentration of 111 nM (except for mAb1-1371 that was added at 55 nM and mAb1-0021 that was added at 166 nM) to Haemophilia A (HA) plasma (George King). Then kaolin mixed with phospholipids (Rossix, #PL604T) was added, followed by addition of FIIa substrate (FluCa, Thrombinoscope, #TS50.00). Fluorescence was measured on a Perkin Elmer EnVision multi-label plate reader at 1 minute intervals for 2 hours. Peak height was calculated as the maximum value observed in the thrombogram, and then normalized to the peak height observed for a reference anti-FIXa and anti-FX antibody. The reference always included a binding domain from the anti-FX antibody mAb1-2375 (identified by SEQ ID NO:93 and 94), in combination with the FIX domains from each of the three families, represented by mAb1-4707, mAb1-5788 and mAb1-4857. Antibodies were grouped according to their relative TGT-activity as low (0-24%), + (24-50%), ++ (50-75%) and +++ (>75%), where + is preferred, ++ is more preferred and +++ is most preferred.

### Selection of Preferred Combinations of Bispecific Anti-FIXa/Anti-FX Antibodies

A large number of anti-FX antibodies were tested as bispecific antibodies, in combination with anti-FIXa antibody variants belonging to the three lineages mAb1-1307, mAb0-1886 and mAb0-1998. Selected combinations of anti-FIXa/anti-FX pairs showing significant activity in the TGT assay are shown in Table 5.

TABLE 5
Procoagulant activity of bispecific antibodies
Selected pairs of anti-FIXa/anti-FX bispecific
antibodies are listed together with their activity
in the TGT-assay (as described above). Bispecific
antibodies (Duobody) were of the IgG4 sub-type, except
for mAb1-0021, mAb1-1335 and mAb1-0985, which were IgG1.

| | FIXa mAb | | |
|---|---|---|---|
| FX mAb | family | Activity | FIXa mAb |
| 1-6705 | 1-1307 | +++ | 1-4707 |
| 1-6716 | 1-1307 | + | 1-4707 |
| 1-6721 | 1-1307 | + | 1-4707 |
| 1-6723 | 1-1307 | + | 1-4707 |
| 1-6731 | 1-1307 | low | 1-4707 |
| 1-6737 | 1-1307 | + | 1-4707 |
| 1-1371 | 1-1307 | +++ | 1-4707 |
| 1-6705 | 0-1886 | ++ | 1-5788 |
| 1-6716 | 0-1886 | ++ | 1-5788 |
| 1-6723 | 0-1886 | + | 1-5788 |
| 1-6731 | 0-1886 | ++ | 1-5788 |
| 1-6737 | 0-1886 | ++ | 1-5788 |
| 1-7378 | 0-1886 | ++ | 1-5788 |
| 1-7413 | 0-1886 | ++ | 1-5788 |
| 1-7441 | 0-1886 | +++ | 1-5788 |
| 1-7447 | 0-1886 | +++ | 1-5788 |
| 1-7449 | 0-1886 | ++ | 1-5788 |
| 1-7462 | 0-1886 | ++ | 1-5788 |
| 1-7466 | 0-1886 | + | 1-5788 |
| 1-7481 | 0-1886 | +++ | 1-5788 |
| 1-1371 | 0-1886 | +++ | 1-4071 |
| 1-0021 | 0-1886 | low | 1-1335 |
| 1-6705 | 0-1998 | ++ | 1-4857 |
| 1-6716 | 0-1998 | +++ | 1-4857 |
| 1-6723 | 0-1998 | +++ | 1-4857 |
| 1-6730 | 0-1998 | +++ | 1-4857 |
| 1-6731 | 0-1998 | +++ | 1-4857 |
| 1-6737 | 0-1998 | +++ | 1-4857 |
| 1-6754 | 0-1998 | +++ | 1-4857 |
| 1-7378 | 0-1998 | +++ | 1-4857 |
| 1-7388 | 0-1998 | + | 1-4857 |
| 1-7413 | 0-1998 | ++ | 1-4857 |
| 1-7424 | 0-1998 | ++ | 1-4857 |
| 1-7441 | 0-1998 | +++ | 1-4857 |
| 1-7447 | 0-1998 | +++ | 1-4857 |
| 1-7449 | 0-1998 | ++ | 1-4857 |
| 1-7462 | 0-1998 | ++ | 1-4857 |
| 1-7466 | 0-1998 | ++ | 1-4857 |
| 1-7481 | 0-1998 | +++ | 1-4857 |
| 1-7483 | 0-1998 | ++ | 1-4857 |
| 1-7563 | 0-1998 | ++ | 1-4857 |
| 1-7571 | 0-1998 | ++ | 1-4857 |
| 1-7591 | 0-1998 | ++ | 1-4857 |
| 1-1371 | 0-1998 | +++ | 1-4857 |
| 1-0021 | 0-1998 | low | 1-0985 |

As evident from Table 5 the level of activity exhibited by the bispecific antibody is dependent on the specific anti-FIXa/anti-FX combination. For example, the anti-FX antibody mAb1-6723 in combination with the anti-FIXa antibody mAb0-1998 exhibits strong activity (+++), whereas the activity of mAb1-6723 is lower in combination with mAb0-1886 (+) and with mAb1-1307 (+).

### Example 12: Binning of Anti-FX Antibodies

Certain anti-FX antibodies showing significant TGT activity in a bispecific antibody format in combination with an anti-hFIXa antibody (Example 11) were binned against each other using the Octet fortebio systems using the same setup as described for anti-FIXa antibodies (Example 4), except for substituting FIXa with FX.

The analysis identified five different bins, Bin A-E, defined by the antibodies mAb1-1371, mAb1-1376, mAb1-6723, mAb1-7447 and mAb1-7449, respectively. Two bins, Bin A and Bin E, are each represented only by a single anti-FX antibody (see Table 6).

A large number of clones were identified in Bin C as competing with mAb1-6723.

TABLE 6
Binning of anti-FX antibodies
A selection of anti-FX antibodies were binned
against each other, and five different bins
(Bin A-E) were identified. Numbers refer to
antibody ID, e.g. 1-6723 denotes mAb1-6723.

| Bin A | Bin B | Bin C | Bin D | Bin E |
|---|---|---|---|---|
| (1-1371) | (1-1376) | (1-6723) | (1-7447) | (1-7449) |

| | | |
|---|---|---|
| 1-6705 | | |
| 1-7388 | 1-7388 | |
| 1-7563 | 1-7563 | |
| | 1-6716 | |
| | 1-6721 | |
| | 1-6730 | |
| | 1-6731 | |
| | 1-6737 | |
| | 1-6754 | |
| | 1-7378 | |
| | 1-7413 | |
| | 1-7424 | |
| | 1-7466 | |
| | 1-7481 | |
| | 1-7483 | |
| | 1-7591 | |
| | 1-7462 | 1-7462 |
| | 1-7571 | 1-7571 |
| | | 1-7441 |

### Example 13: Crystallization and Epitope Mapping of Anti-FX Antibody Using X-Ray Crystallography

### Crystallisation

Attempts to crystallize Fab0-8954 (Fab fragment corresponding to mAb1-6723) in complex with FX were unsuccessful, whereas good quality crystals with active-site inhibited FXa were obtained. Thus, crystals of Fab0-8954 mixed in a 1:1 molar ratio with active site inhibited des-gla FXa (human EGR-inhibited Factor Xa gla-domainless (wild-type) bacterial expression, Lot #hGDFXAEGR-022, Cambridge ProteinWorks) were grown using the sitting drop vapour diffusion technique at 18 00. A protein solution of 150 nl 6.7 mg/ml complex in 20 mM Tris-HCl, pH 7.4, 50 mM NaCl, and 2.5 mM CaCl₂ was mixed with 50 nl of 0.2 M magnesium acetate, 0.1 M sodium cacodylate, pH 6.5, 20% (w/v) PEG 8000 as precipitant and incubated over 60 μl precipitant.

### Diffraction Data Collection

The crystal was cryo-protected by addition of 1 μl of precipitant added 20% of ethylene glycol to the crystallisation drop prior to flash cooling in liquid nitrogen. Diffraction data were collected at 100K at the Swiss Light Source beamline X06DA (1.0000 Å wavelength) using a Pilatus2M pixel detector from Dectris. Autoindexing, integration and scaling of the data were performed with programmes from the XDS package (diffracting data statistics are summarised in Table 7).

### Structure Determination and Refinement

The asymmetric unit contains four Fab:FXa complexes as judged from Matthews coefficient analysis. The structure was determined by molecular replacement. Phaser as implemented in the programme suite Phenix was used with the chains H and L of protein data bank entry 5I1K as search model localising four Fabs. These were model built with the correct amino acid sequence using COOT and thereafter refined using Phenix refinement. The refined Fab model was fixed while applying molecular replacement in Molrep from the CCP4 suite with chains A and B from protein data bank entry 1 G2L as search model. Four FXa fragments were found. The model was refined using steps of Phenix refinement and manual rebuilding in COOT. The refinement statistics are found in Table 7.

TABLE 7
Data collection and refinement statistics

| | |
|---|---|
| Wavelength (Å) | 1.0000 |
| Resolution range (Å) | 78.09-2.873 (2.976-2.873) |
| Space group | P2₁ |
| Unit cell (Å, deg) | 123.3 91.9 156.2 90 90.5 90 |
| Total reflections | 269989 (27782) |
| Unique reflections | 78735 (7779) |
| Multiplicity | 3.4 (3.6) |
| Completeness | 98.44 (98.49) |
| Mean I/sigma(I) | 9.66 (1.23) |
| Wilson B-factor (Å²) | 58.38 |
| R-merge | 0.1398 (1.152) |
| R-meas | 0.166 (1.355) |
| R-pim | 0.0885 (0.7084) |
| CC1/2 | 0.994 (0.519) |
| CC* | 0.999 (0.826) |
| Reflections used in refinement | 78688 (7778) |
| Reflections used for R-free | 2000 (201) |
| R-work | 0.2093 (0.3449) |
| R-free | 0.2780 (0.4405) |
| CC(work) | 0.929 (0.712) |
| CC(free) | 0.911 (0.270) |
| Number of non-hydrogen atoms | 22125 |
| macromolecules | 22021 |
| ligands | 104 |
| Protein residues | 2868 |
| RMS(bonds) (Å) | 0.012 |
| RMS(angles) (deg) | 1.64 |
| Ramachandran favored (%) | 95.04 |
| Ramachandran allowed (%) | 4.11 |
| Ramachandran outliers (%) | 0.85 |
| Rotamer outliers (%) | 0.16 |
| Clashscore | 12.20 |
| Average B-factor (Å²) | 60.54 |
| macromolecules | 60.55 |
| ligands | 59.50 |

Statistics for the highest-resolution shell are shown in parentheses.

### Determination of Epitope

The crystal structure of the Fab0-8954:FXa complex has four copies of the complex in the asymmetric unit and these were analysed separately to identify epitope and paratope using a 3.5 Å cut-off distance.

Residues are included in the epitope for mAb1-6723 if satisfying the 3.5 Å distance criteria in at least one of the four copies of the Fab0-8954:FXa complex in the unit cell. Epitope and paratope residues for mAb1-6723 are listed in table 8.

TABLE 8
Epitope and paratope for mAb1-6723
Epitope residues for mAb1-6723 in the EGF-2
and protease domain of FX/FXa (SEQ ID NO: 2)
are listed in first and second columns,
respectively. Paratope residues in antibody V_{H}
(SEQ ID NO: 21) and V_{L} (SEQ ID NO: 22)
are listed in columns three and four, respectively.

| Residues in | Residues in | | |
|---|---|---|---|
| EGF-2 domain | protease domain | V_{H} | V_{L} |
| H101, E103, | S227, E228, F229, | S25, G26, Y27, | S30, S31, S32, |
| R113, T116, | Y230, E266, R287, | S28, F29, T31, | Y33, Y50, S54, |
| L117, A118 | L303, P304, E305, | W33, D52, S54, | R55, R57 and |
| and T127 | L419, K420, D423, | D55, F57, S77, | R96 |
| | R424, M426, K427 | H100, Y101, | |
| | and T428 | Y102, H103, | |
| | | S104 and E106 | |

Variants of Parental Antibodies (Lineages) as Disclosed Herein Share Bins and Epitope (Hot-Spot) Residues with Parental Antibodies.

Since the antibody variants for which data are provided in the examples herein do not contain amino acid substitutions on positions shown to be crucial for epitope recognition based on the crystal structures of the parental antibody-FXa complexes provided in the present example, a person skilled in the art would understand that the variants as a starting point will belong to the same bin, compete for binding with, and recognize at least the same hot-spot residues in the FX/FXa epitope as the antibody from which they originate, i.e. mAb1-6723.

### Example 14: Identification of Hot-Spot Residues on FX

Similar to the mapping of hot-spot epitope residues on FIX for mAb1-1307, mAb0-1886 and mAb0-1998, as described in example 15, the data provided in the present example determines the hot-spot epitope residues on FX for mAb1-6723. The FX variants used were single-site alanine variants (except for position 118, which is alanine in the wild-type, where an alanine to serine substitution was introduced) of desGla-desEGF1-FX, corresponding to residues 86-448 of SEQ ID NO:2 with a N-terminal His-tag (HHHHHH, for affinity purification) attached via a short GS-linker (GGGGSGGGGS). The variants covering epitope residues as defined in example 13 are listed in table 9.

TABLE 9
List of generated desGla-desEGF1-FX variants

| Position¹⁾ | Variant | Domain |
|---|---|---|
| 101 | H101A | EGF2 |
| 103 | E103A | EGF2 |
| 113 | R113A | EGF2 |
| 116 | T116A | EGF2 |
| 117 | L117A | EGF2 |
| 118 | A118S | EGF2 |
| 127 | T127A | EGF2 |
| 227 | S227A | PD |
| 228 | E228A | PD |
| 229 | F229A | PD |
| 230 | Y230A | PD |
| 266 | E266A | PD |
| 287 | R287A | PD |
| 303 | L303A | PD |
| 304 | P304A | PD |
| 305 | E305A | PD |
| 419 | L419A | PD |
| 420 | K420A | PD |
| 423 | D423A | PD |
| 424 | R424A | PD |
| 426 | M426A | PD |
| 427 | K427A | PD |
| 428 | T428A | PD |

¹⁾According to SEQ ID No: 2
²⁾ EGF2 and PD refer to second epidermal growth factor-like and protease domains, respectively

The wild-type desGla-desEGF1-FX and variants listed in table 9 were expressed in the HEK293 system and purified via affinity chromatography. No expression or poor purity was observed for the L117A, L303A, P304A and M426A variants, and assessment of binding was not possible for those four variants.

Identification of hot-spot epitope residues was done using a Biacore T200 instrument at 25° C. Anti-hIgG Fc antibody from the Human Antibody Capture Kit (GE Healthcare, Catalogue #BR100839) at 2 μg/ml was immobilized on a Series S Sensor Chip CM5 (GE Healthcare, Catalogue #BR100530) using standard amine coupling chemistry. The anti-FX antibody mAb4-6934 (monovalent variant of mAb1-6723) was injected at the flow rate of 5 μL/min for 30 sec and captured by the immobilized anti-hIgG Fc antibody. Subsequently, 5 μM (with 2 or 3-fold serial dilutions) of the T116A, A118S, T127A, F229A and E226A variants, 10 μM (with 5-fold serial dilutions) of the Y230A variant, and 10 μM (with 2 or 3-fold serial dilutions) WT and H101A, E103A, R113A, S227A, E228A, R287A, E305A, L419A, K420A, D423A, R424A, K427A and T428A variants were injected at the flow rate of 5 μL/min for 90 sec to allow for binding to the captured anti-FX antibody followed by a 90 sec buffer injection to allow for dissociation of the desGLA-desEGF1-FX variants. The running buffer (also used for diluting the anti-FX antibody and desGLA-desEGF1-hFX variants) contained 10 mM HEPES, 150 mM NaCl, 1 mg/mL BSA and 5 mM CaCl₂ (pH 7.4). Regeneration of the sensor chip was achieved using 1 M formic acid. Binding data were analyzed using steady-state fitting according to the 1:1 model in the Biacore Evaluation Software 2.0 supplied by GE Healthcare. Binding data are reported as % binding of FX variants to the anti-FX antibody (monovalent mAb1-6723) relative to binding of the wild-type FX to the anti-FX antibody at 5 or 10 μM FX variants injected and are calculated according to the formula:

$$Binding ( \% ) = 100 \% \times [ ( {R}_{max \_ FXvar , Ab} ) / ( {R}_{max \_ Ab} ) ] / [ ( {R}_{max \_ FXwt , Ab} ) / ( {R}_{max \_ Ab} ) ]$$

where R_{max_Ab} represents the capture level (RU) of the anti-FX antibody, and R_{max_FXvar,Ab} and R_{max_FXwt,Ab} represent the binding (RU) of FX variants and wild-type at the same concentration (5 μM for all except for Y230A variant, where the concentration was 10 μM) to the captured anti-FX antibody, respectively. Results are shown in table 10.

TABLE 10
Results from SPR analysis
Results from SPR analysis of monovalent variant of mAb1-6723 binding
to selected FX variants covering epitope residues for mAb1-6723.

| | Binding |
|---|---|
| Variant | (%) |
| WT | 100 |
| H101A | 51 |
| E103A | 71 |
| R113A | 28 |
| T116A | 114 |
| A118S | 102 |
| T127A | 113 |
| S227A | 66 |
| E228A | 80 |
| F229A | 99 |
| Y230A | 1 |
| E266A | 112 |
| R287A | 66 |
| E305A | 97 |
| L419A | 50 |
| K420A | 23 |
| D423A | <1 |
| R424A | <1 |
| K427A | <1 |
| T428A | 81 |

Hot-Spot Residues for mAb1-6723

Hot-spot residues for mAb1-6723 are defined as positions where substitution of the wild-type residue with alanine (or for position 118 substitution of alanine with serine) reduces the binding of the antibody to 30% or less relative to binding of the antibody to wild-type FX at a concentration of 5 μM of WT (or variant) desGLA-desEGF1-FX.

Hot-spot residues for mAb1-6723 (experimentally represented by its monovalent counterpart, mAb 4-6934):

### R113, Y230, K420, D423, R424 and K427

### Example 15: Identification of Hot-Spot Residues on FIX/FIXa

In order to determine residues critical for the interaction (referred to as hot-spot) between the anti-FIX/FIXa Abs, mAb0-1886, mAb0-1998 and mAb1-1307 and FIX, a set of FIX variants was selected based on the crystal structure of FIXa in complex with the corresponding Fab fragments (Fab7237, Fab7238 and Fab7236, respectively). As detailed below the selected FIX variants were transiently expressed in mammalian cells, purified and characterized with respect to their binding to monovalent variants of mAb0-1886, mAb0-1998 and mAb1-1307 using Surface Plasmon Resonance (SPR).

### Generation of FIX Mutants

A DNA plasmid, suitable for transient mammalian expression, was constructed with an expression cassette encoding amino acids residues 1-461 of human FIX (uniprot P00740, except for a T194A mutation according to the UNIPROT numbering, corresponding to T148A of SEQ ID NO:1) directly followed by six Histidines (6×His-tag, for affinity purification). The secreted, mature FIX protein chain produced using this construct is identical to the A148 allelic form of human FIX (Anson et al. EMBO J. 1984 3:1053-1060, McGraw et al, Proc Natl Acad Sci USA. 1985 82:2847-2851) except for the addition of the C-terminal His-tag.

Using the construct as template, selected mutations were introduced by PCR. For each single-point mutation listed in Table 11, a forward primer containing the desired amino acid change and a reverse primer without amino acid mutations were designed. These primers were used in a standard PCR reaction with the vector described above as template to amplify the entire vector sequence. Ligation-free cloning was used to join the ends of the resulting amplified DNA fragment into a circular expression plasmid using overlap sequences introduced by the forward and the reverse primers.

The circularized plasmids were transformed into E. coli cells, grown on selective agar plates to form colonies, and the colonies used to start liquid E. coli cultures. After overnight growth of the E. coli cultures, plasmid preparations were performed and the mutants identified by DNA sequencing.

Recombinant protein production was performed by transfecting expi293F cells growing in suspension culture in Expi293 Expression™ medium (ThermoFisher Scientific, cat #A1435101) using the ExpiFectamine™ 293 Transfection Kit (ThermoFisher Scientific, cat #A14525) and plasmid DNA encoding each of the desired variants as well as wild-type FIX (corresponding to SEQ ID NO:1 with C-terminal His-tag). Vitamin K was added to a final concentration of 5 mg/mL at the time of transfection. Transfection Enhancers 1 and 2 from the ExpiFectamine™ 293 Transfection Kit were added the day after transfection. The cell cultures were harvested 5 days after transfection by centrifugation.

The C-terminal His-tag on each FIX variant was used for batch protein purification in a multi-well, robotic setup. Briefly, the harvested cell culture supernatants were adjusted to binding conditions, mixed with Ni Sepharose 6 Fast Flow affinity purification resin (GE Healthcare, cat #17-5318-02, 50 μl sedimented resin/ml cell culture medium) and incubated while shaking for 20 minutes. The resin/supernatant mixes were then transferred to a filter plate and the liquid drawn through the filter plate by application of vacuum. The resin remaining in the filter plate was washed three times before elution in a high-imidazole buffer.

Concentration determination of the purified protein solutions was performed by ELISA, using an anti-FIX antibody for detection and high-purity recombinant wild-type FIX for standard curves.

TABLE 11
List of generated FIX mutants

| Position¹⁾ | Mutation | Domain²⁾ |
|---|---|---|
| 84 | L84K | EGF2 |
| 84 | L84M | EGF2 |
| 84 | L84E | EGF2 |
| 85 | D85Q | EGF2 |
| 85 | D85K | EGF2 |
| 85 | D85N | EGF2 |
| 87 | T87E | EGF2 |
| 87 | T87I | EGF2 |
| 89 | N89M | EGF2 |
| 89 | N89Q | EGF2 |
| 90 | I90Y | EGF2 |
| 90 | I90A | EGF2 |
| 101 | N101D* | EGF2 |
| 102 | S102A | EGF2 |
| 102 | S102E | EGF2 |
| 102 | S102R | EGF2 |
| 256 | H256F | PD |
| 256 | H256A | PD |
| 257 | H257F | PD |
| 257 | H257A | PD |
| 258 | N258Q | PD |
| 258 | N258A | PD |
| 263 | I263A | PD |
| 292 | D292N | PD |
| 292 | D292S | PD |
| 293 | K293A | PD |
| 294 | E294A | PD |
| 294 | E294Q | PD |
| 301 | K301A | PD |
| 330 | L330A | PD |
| 331 | V331A | PD |
| 331 | V331I | PD |
| 332 | D332A | PD |
| 332 | D332S | PD |
| 333 | R333A | PD |
| 334 | A334L | PD |
| 335 | T335A | PD |
| 337 | L337A | PD |
| 338 | R338A | PD |
| 339 | S339L | PD |
| 339 | S339A* | PD |
| 340 | T340A | PD |
| 341 | K341A | PD |
| 341 | K341E | PD |
| 342 | F342A | PD |
| 343 | T343I | PD |
| 343 | T343A | PD |
| 346 | N346A | PD |
| 346 | N346Q | PD |
| 354 | H354Y | PD |
| 354 | H354A | PD |
| 392 | K392E | PD |
| 392 | K392A | PD |
| 392 | D292A | PD |
| 393 | G393I | PD |
| 395 | Y395A | PD |
| 400 | K400M | PD |
| 400 | K400A | PD |
| 402 | S402A | PD |
| 403 | R403Q | PD |
| 403 | R403A | PD |
| 404 | Y404A* | PD |
| 404 | Y404F | PD |
| 405 | V405A | PD |
| 406 | N406Q | PD |
| 406 | N406A | PD |
| 410 | E410Q | PD |
| 411 | K411A | PD |

³⁾ According to SEQ ID NO: 1
⁴⁾ EGF2 and PD refer to second epidermal growth factor-like and protease domains, respectively
*Variants marked with an asterisk exhibited very low expression levels and could not be assessed in binding studies

### Thermal Stability of FIX Variants

To test if introduction of the amino acid substitutions in the FIX variants lead to destabilization and improper folding, the midpoint (Tₘ) of the thermal unfolding transition was determined for the variants.

Purified FIX variants were loaded into standard capillaries (Prometheus NT.48 nanoDSF Grade Standard capillaries, Nanotemper Technologies GmbH, München) and inserted in to the Prometheus NT.48 (Nanotemper Technologies GmbH, München). An excitation intensity of 70% was used and thermal unfolding was followed from 20-90° C. with a heating ramp of 1.5° C./min. Tryptophan fluorescence was measured by excitation at 280 nm and recording emission at 330 nm and 350 nm. Tₘ of the FIX variants could be determined (except where the protein concentration was below 20 μg/mL, which was the case for FIX N101 D, H256A, L330A S339A, G393I, Y404A and N406Q) from the ratio of fluorescence measured at 350 nm and 330 nm (F350/F330). In all cases, the program PR.ThermControl v2.0.4 (NanoTemper Technologies GmbH, München) could automatically fit Tₘ by determining the maximum of the first derivative of the F350/F330 unfolding curve. Tₘ for wild-type FIX was found to be 51° C. and Tₘ for the variants ranged from 47 to 54° C. demonstrating that no major destabilization was induced by the amino acid substitutions.

### SPR Analysis

The FIX variants were characterized with respect to their binding to mAb0-1886, mAb0-1998 and mAb1-1307 using surface plasmon resonance (SPR) by capturing the FIX variant via the C-terminal His-tag. To avoid potential avidity effects associated with a conventional bivalent antibody, i.e. ensure a 1:1 interaction, monovalent variants of mAb0-1886, mAb0-1998 and mAb1-1307, denoted mAb4-0673, mAb4-0004 and mAb3-3279, respectively (prepared as described in Example 7), were used as analytes.

SPR analyses were carried out on Biacore 4000 or Biacore T200 instruments (Biacore AB, Uppsala, Sweden). For the experiments on the T200 instrument the following conditions were applied: measurements were conducted at a temperature of 25° C. Anti-His antibody at 25 μg/ml (R&D Systems, catalogue #MAB050) was immobilized on a CM5 sensor chip using standard amine coupling chemistry. Anti-FIX variants at 25 nM were injected at a flow rate of 10 μl/min for 1 min and were captured via their His-tag by the immobilized anti-His antibody. Subsequently, 200 nM (with 4-fold serial dilutions), 1600 nM (with 3-fold serial dilutions), and 2000 nM (with 3-fold serial dilution) of mAb4-0004, mAb3-3279 and mAb4-0673, respectively, were injected at a flow rate of 50 μl/min for 5 min to allow for binding to captured FIX variant followed by a 10 min buffer injection allowing for dissociation of the monovalent anti-FIX antibodies. The running buffer used was 20 mM Tris, 150 mM NaCl, 5 mM CaCl₂, 0.05% Tween-20, 1 mg/ml BSA, pH 7.4. This was also used for dilution of anti-FIX antibody and FIX samples. Regeneration of the chip was achieved using 10 mM Glycine pH 2.0. Binding data were analysed according to a 1:1 model using BiaEvaluation 4.1 supplied by the manufacturer (Biacore AB, Uppsala, Sweden). A similar experimental set-up was used for the Biacore 4000 instrument.

Initially all FIX variants listed in table 11 were screened using the Biacore 4000 instrument for binding to all three monovalent antibodies, mAb4-0004, mAb3-3279 and mAb4-0673. Binding of the antibodies to FIX variants comprising mutations in positions corresponding to their respective epitope residues (defined by a distance criterion as outlined in example 6) were, as expected, to variable extent perturbed. No significant impact on antibody binding was observed for FIX variants comprising mutations in position not corresponding to their respective epitope residues. In particular, none of the substitutions made in the EGF2 domain had any influence on binding to any of the antibodies (data not shown).

A more detailed binding analysis was conducted for residues defined as epitope residues (see example 6) using the Biacore T200 instrument. Results are given in table 12.

Binding data are reported as % binding of the antibody to the FIX variant relative to binding of the antibody to wild-type FIX calculated according to the formula:

$$Binding ( \% ) = 100 \% \times [ ( {R}_{max \_ Ab , FIX \_ var} ) / ( {R}_{max \_ FIXvar} ) ] / [ ( {R}_{max \_ Ab , FIX \_ wt} ) / ( {R}_{max \_ FIXwt} ) ]$$

where R_{max_FIXvar} and R_{max_FIXwt} represent capture level (RU) of FIX variant and wild-type FIX, respectively, and where R_{max_Ab,FIX_var} and R_{max_Ab,FIX_wt} represent binding (RU) of the antibody to captured FIX variant and wild-type FIX, respectively. Results are shown in table 12.

TABLE 12
Results from SPR analysis
Results from SPR analysis of mAb3-3279, mAb4-0004,
and mAb4-0673 (monovalent variants of mAb1-1307,
mAb0-1998 and mAb0-1886, respectively) binding to
selected FIX variants covering epitope residues
for mAb1-1307, mAb0-1998 and mAb0-1886.

| | | | | Binding |
|---|---|---|---|---|
| Lineage | Position | Variant | Antibody | (%) |
| 1-1307 | 256 | H256A | mAb3-3279 | 32 |
| 1-1307 | 257 | H257A | mAb3-3279 | 8 |
| 1-1307 | 258 | N258A | mAb3-3279 | 82 |
| 1-1307 | 293 | K293A | mAb3-3279 | 23 |
| 1-1307 | 403 | R403A | mAb3-3279 | 94 |
| 1-1307 | 404 | Y404F | mAb3-3279 | 76 |
| 1-1307 | 406 | N406A | mAb3-3279 | 12 |
| 1-1307 | 410 | E410Q | mAb3-3279 | 39 |
| 1-1307 | 411 | K411A | mAb3-3279 | 71 |
| 1-1307 | WT | WT | mAb3-3279 | 100 |
| 0-1998 | 301 | K301A | mAb4-0004 | 66 |
| 0-1998 | 332 | D332S | mAb4-0004 | 66 |
| 0-1998 | 332 | D332A | mAb4-0004 | 50 |
| 0-1998 | 333 | R333A | mAb4-0004 | 66 |
| 0-1998 | 334 | A334L | mAb4-0004 | 51 |
| 0-1998 | 335 | T335A | mAb4-0004 | 62 |
| 0-1998 | 337 | L337A | mAb4-0004 | 52 |
| 0-1998 | 338 | R338A | mAb4-0004 | 2 |
| 0-1998 | 339 | S339L | mAb4-0004 | 66 |
| 0-1998 | 340 | T340A | mAb4-0004 | 37 |
| 0-1998 | 341 | K341E | mAb4-0004 | 2 |
| 0-1998 | 341 | K341A | mAb4-0004 | 21 |
| 0-1998 | 343 | T343I | mAb4-0004 | 48 |
| 0-1998 | 343 | T343A | mAb4-0004 | 38 |
| 0-1998 | 346 | N346A | mAb4-0004 | 73 |
| 0-1998 | WT | WT | mAb4-0004 | 100 |
| 0-1886 | 301 | K301A | mAb4-0673 | 93 |
| 0-1886 | 332 | D332A | mAb4-0673 | 17 |
| 0-1886 | 333 | R333A | mAb4-0673 | 4 |
| 0-1886 | 334 | A334L | mAb4-0673 | 44 |
| 0-1886 | 335 | T335A | mAb4-0673 | 71 |
| 0-1886 | 337 | L337A | mAb4-0673 | 7 |
| 0-1886 | 338 | R338A | mAb4-0673 | 3 |
| 0-1886 | 339 | S339L | mAb4-0673 | 73 |
| 0-1886 | 340 | T340A | mAb4-0673 | 67 |
| 0-1886 | 341 | K341E | mAb4-0673 | 53 |
| 0-1886 | 341 | K341A | mAb4-0673 | 96 |
| 0-1886 | 343 | T343I | mAb4-0673 | 90 |
| 0-1886 | 343 | T343A | mAb4-0673 | 63 |
| 0-1886 | 346 | N346Q | mAb4-0673 | 94 |
| 0-1886 | 346 | N346A | mAb4-0673 | 51 |
| 0-1886 | WT | WT | mAb4-0673 | 100 |

1) Position according to SEQ ID NO: 1
2) 100% × [(Rₘₐₓ_Ab, FIX_var)/(Rₘₐₓ_FIXvar)]/[(Rₘₐₓ_Ab, FIX_wt)/(Rₘₐₓ_FIXwt)]

Hot-Spot Residues mAb1-1307, mAb0-1998 and mAb0-1886

Hot-spot residues for mAb1-1307, mAb0-1998 and mAb0-1886 are defined as positions were substitution of the wild-type residue with alanine reduces the binding of the antibody to 30% or less relative to binding of the antibody to wild-type FIX.

Hot-spot residues for mAb1-1307 (experimentally represented by mAb3-3279): H257, K293 and N406

Hot-spot residues for mAb0-1998 (experimentally represented by mAb4-0004): R338 and K341

Hot-spot residues for mAb0-1886 (experimentally represented by mAb4-0673): D332, R333, L337 and R338

For both mAb0-1998 and mAb0-1886 the residue contributing most to binding is R338; substitution of R338 with alanine (R338 Å) in FIX exhibited the largest impact on antibody binding, which was greatly reduced to 2% and 3% for mAb0-1998 and mAb0-1886, respectively, relative to antibody binding to wild-type FIX.

### Example 16: Activity of Anti-FIX(a)/FX(a) Bispecific Antibodies in a FXa Generation Assay

The procoagulant activity of anti-FIXa/FX bispecific antibodies was determined based on their ability to promote FX activation by FIXa in the presence of a procoagulant phospholipid membrane. The bispecific antibodies (BiAb) tested are listed in table 13 and ACE910 was included for comparison.

The procoagulant activity of each bispecific antibody is reported as fold stimulation relative to FX activation by free FIXa at a given antibody concentration. Bispecific antibodies were tested at 8 concentrations (made by serial three-fold dilutions in assay buffer) by pre-incubation with 125 μM human plasma-derived FIXa (Haematologic Technologies Inc, USA) and 500 μM 25:75 phosphatidyl serine:phosphatidyl choline phospholipid vesicles (Haematologic Technologies Inc, USA) in assay buffer (50 mM HEPES, 100 mM NaCl, 5 mM CaCl₂, 0.1% (w/v) PEG8000, pH 7.3+1 mg/ml BSA) for 10 min. Activation was then initiated by addition of human plasma-derived FX (Haematologic Technologies Inc, USA) to a concentration of 25 nM. Following 15 min activation at room temperature, the reaction (50 μl) was quenched by addition of 25 μl quench buffer (50 mM HEPES, 100 mM NaCl, 60 mM EDTA, 0.1% PEG8000, pH 7.3+1 mg/ml BSA). The amount of FXa generated was determined by addition of 25 μl 2 mM S-2765 chromogenic substrate (Chromogenix, Sweden) and measurement of chromogenic substrate conversion by absorbance measurement at 405 nm (ΔOD/min) in a microplate reader. Similarly, FX activation by free FIXa was determined at a FIXa concentration of 25 nM and a reaction time of 60 min.

The measured activity was normalized according to the concentration of FIXa present in the assay and the reaction time. By dividing this number by the similarly normalized rate of FXa generation in the absence of antibody, fold stimulation by the antibody at a given concentration was calculated.

In summary, calculation of biAb stimulation can be described as follows

$$BiAb stimulation = ( {A}_{FIXa + biAb} / ( {[ FIXa ]}_{assay} \times {t}_{reaction} ) ) / {A}_{FIXa , norm}$$

where A_{FIXa+biAb} is the activity measured in the presence of bispecific antibody, [FIXa]_{assay} is the FIXa concentration in the assay, t_{reaction} is the reaction time, and A_{FIXa,norm} is the normalized activity of free FIXa.

Table 13 lists the maximum stimulation determined for each bispecific antibody among the 8 antibody concentrations tested as well as the concentration at which maximum stimulation was observed. For all tested bispecific antibodies the maximum stimulation was found to be higher than that measured for ACE910, which was tested at a concentration interval from 0 to 15300 nM.

TABLE 13
Maximum stimulation by bispecific anti-FIXa/FX antibodies

| | FIXa | FX | Concentration | BiAb conc | Maximum |
|---|---|---|---|---|---|
| BiAb | antibody ID | antibody ID | span tested | at maximum | stimulation |
| antibody ID | (lineage) | (lineage) | (nM) | stimulation (nM) | (fold) |
| ACE910 | | | 7.5-15300 | 15300 | 808 |
| 5-0057 | 1-8768 | 1-6723 | 1.7-3654 | 1218 | 10754 |
| | (0-1998) | (1-6723) | | | |
| 5-1409 | 1-8768 | 1-7503 | 1.5-3346 | 1115 | 11041 |
| | (0-1998) | (1-6723) | | | |
| 4-7687 | 1-6037 | 1-6723 | 1.6-3300 | 1650 | 2493 |
| | (0-1998) | (1-6723) | | | |
| 4-7756 | 1-6584 | 1-6723 | 0.7-1520 | 1520 | 2597 |
| | (0-1886) | (1-6723) | | | |
| 4-7758 | 1-6584 | 1-6097 | 0.6-1266 | 633 | 2807 |
| | (0-1886) | (1-2375) | | | |
| 4-7762 | 1-6584 | 1-6738 | 0.1-109.2 | 109 | 3194 |
| | (0-1886) | (1-2375) | | | |
| 4-7786 | 1-6081 | 1-6463 | 2.0-4060 | 4060 | 1267 |
| | (0-1998) | (1-2375) | | | |
| 4-7789 | 1-6584 | 1-6463 | 1.0-2020 | 253 | 3529 |
| | (0-1886) | (1-2375) | | | |
| 4-5925 | 1-4857 | 1-6723 | 0.7-1600 | 533 | 5195 |
| | (0-1998) | (1-6723) | | | |

### Example 17: Activity of Bispecific Anti-FIX(a)/FX(a) Antibodies in a Thrombin Generation Test (TGT) in Human Haemophilia a Platelet-Poor and Platelet-Rich Mimic Plasma

The procoagulant activity of the bispecific antibodies mAb4-7761, mAb4-7762, mAb4-7789, mAb5-0057, and mAb5-1409 (see Table 14) was determined based on their ability to promote thrombin generation in the presence of either a procoagulant synthetic phospholipid membrane or platelets according to the principles described by Hemker et al. (Pathophysiol Haemost Thromb, 2002; 32:249-253). ACE910 was included for comparison. Each antibody (test compound) was tested in a thrombin generation test (TGT) in Haemophilia A (HA) patient pooled platelet-poor plasma (HA-PPP) and/or HA-induced human platelet-rich plasma (HA-PRP).

TABLE 14
Bispecific anti-FIX(a)/FX(a) antibodies

| | Anti-FIX | Anti-FX |
|---|---|---|
| BiAb | antibody ID | antibody ID |
| antibody ID | (lineage) | (lineage) |
| 4-7761 | 1-5743 | 1-6738 |
| | (0-1886) | (1-2375) |
| 4-7762 | 1-6584 | 1-6738 |
| | (0-1886) | (1-2375) |
| 4-7789 | 1-6584 | 1-6463 |
| | (0-1886) | (1-2375) |
| 5-0057 | 1-8768 | 1-6723 |
| | (0-1998) | (1-6723) |
| 5-1409 | 1-8768 | 1-7503 |
| | (0-1998) | (1-6723) |

### Haemophilia A-Induced Human Platelet-Rich Plasma (HA-PRP)

Blood was obtained from healthy consenting donors by venipuncture. Six volumes of blood was collected into 1 volume acid citrate dextrose (ACD; 85 mM sodium citrate, 110 mM dextrose, and 62.3 mM citric acid, pH 4.9), final pH 6.5, and centrifuged for 20 min at 220 g at room temperature (RT). Platelet-rich plasma (PRP) was collected and platelet concentrations were determined with a Medonic CA 620 hematology analyzer (Boule Diagnostics AB, Spanga, Sweden). The red blood cells containing plasma part was centrifuged for another 10 min at 600 g at RT. Platelet-poor plasma (PPP) was collected and used to the dilute PRP to 300,000 platelets/μl. HA conditions were induced by addition of a FVIII-neutralising anti-human FVIII antibody (Sheep anti-Human Factor VIII—5 mg, Haematologic Technologies, VT, USA) to a final concentration of 0.1 mg/ml and rotated gently at 2 rpm for 30 minutes at RT.

### Thrombin Generation Test

Thrombin generation tests (TGT) in HA-PRP and HA-PPP (George King Bio-Medical Inc, KS, USA) were performed by standard calibrated automated thrombography using a 96-well plate fluorometer (Fluoroscan Ascent FL, Thermolabsystems, Helsinki, Finland). Reaction mixtures contained 70 μl HA-PRP (300,000 platelets/μl) or HA-PPP, 10 μl test compound dilution (diluted in 20 mM HEPES, 140 mM NaCl, pH 7.4, 2% BSA), 20 μl CAT reagents containing tissue factor (TF) (PRP reagent; TF without synthetic phospholipids, PPP-reagent LOW; TF with synthetic phospholipids, 1 pM TF final, Thrombinoscope BV, Maastricht, the Netherlands) or Thrombin Calibrator (Thrombinoscope BV), and 20 μl of a mixture containing the fluorescently labelled thrombin substrate z-Gly-Gly-Arg-AMC (3 mM) and CaCl₂ (90 mM) (Thrombinoscope BV). TGT was performed at up to eight concentrations of test compound (0.3, 1.0, 3, 10, 30, 100, 300, and 900 nM, final plasma concentration) or added buffer (20 mM HEPES, 140 mM NaCl, pH 7.4, 2% BSA) only (representing HA control). The concentration ranges were tested in at least three independent experiments in HA-PPP from the same stock or in blood from four different donors. Normal control levels in TGT were measured using untreated human PRP or CRYOcheck™ pooled normal human PPP plasma (Precision Biologic Inc., Dartmouth, Canada) added buffer (20 mM HEPES, 140 mM NaCl, pH 7.4, 2% BSA) only. The TGT was allowed to proceed for a total of 90 minutes and the TGT parameter Peak Thrombin Height (nM) was analysed by Thrombinoscope software (Thrombinoscope BV).

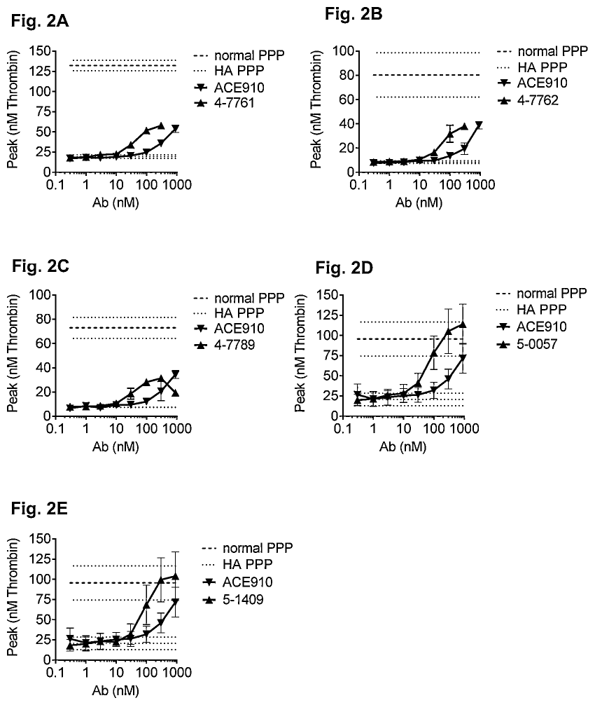

FIG. 2/Table 15 shows the measured peak thrombin generation rates for each bispecific antibody at the concentrations tested in HA-PPP. The data show that all test compounds increase the peak thrombin formation above the level observed in the absence of antibody, i.e. exhibit procoagulant activity. In addition, thrombin generation levels between 30 and 300 nM for mAb4-7761, mAb4-7762, mAb4-7789, mAb5-0057, and mAb5-1409 are higher than that observed for ACE910, demonstrating to a superior potency. Moreover, thrombin generation levels at 300 to 900 nM of mAb5-0057 and mAb5-1409 are higher than that observed with 900 nM ACE910, demonstrating higher potencies and efficacies of these compounds compared to ACE910.

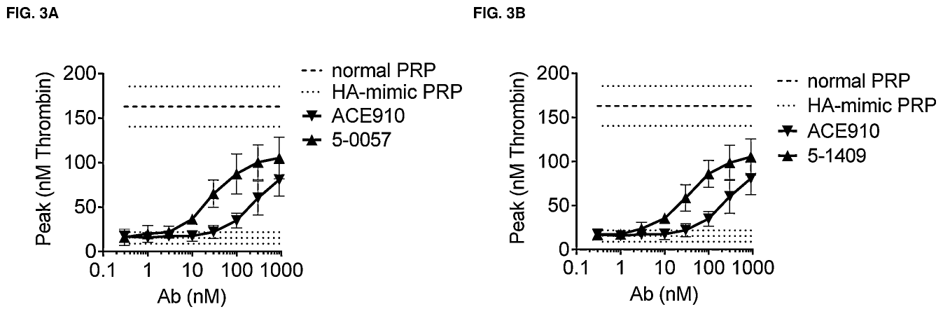

FIG. 3/Table 16 shows the measured peak thrombin generation levels for mAb5-0057 and mAb5-1409 at the concentrations tested in HA-PRP. Under these conditions, mAb5-0057 and mAb5-1409 also display better potencies and efficacies compared to ACE910.

TABLE 15
Thrombin generation test (TGT) of the
bispecific antibodies mAb4-7761, mAb47762,
mAb4-7789, mAb5-0057, mAb5-1409 and ACE910
Thrombin generation test (TGT) of the bispecific
antibodies mAb4-7761, mAb4-7762, mAb4-7789, mAb5-0057,
mAb5-1409 and ACE910 in human tissue factor activated
haemophilia A platelet-poor plasma (PPP). Mean peak
thrombin generation levels ± standard deviation
measured at each of the tested compound concentrations
in at least three independent experiments in HA-PPP.
Exp. A-D refers to independent experiments as
described in the FIG. 2 legend.

| Exp. A | Peak thrombin | | Peak thrombin |
|---|---|---|---|
| Compound | (mean ± | | (mean ± |
| concentration | SD in nM) | | SD in nM) |
| (nM) | for ACE910 | | for mAb4-7761 |
| 0 | 19.4 ± 2.3 | | 19.4 ± 2.3 |
| 0.3 | 17.2 ± 0.4 | | 18.1 ± 2.4 |
| 1 | 18.3 ± 1.4 | | 18.9 ± 0.9 |
| 3 | 17.9 ± 2.1 | | 21.8 ± 3.7 |
| 10 | 19.3 ± 1.0 | | 22.4 ± 1.6 |
| 30 | 20.5 ± 2.3 | | 34.0 ± 1.9 |
| 100 | 24.6 ± 2.0 | | 51.7 ± 3.7 |
| 300 | 35.9 ± 3.0 | | 57.9 ± 1.4 |
| 900 | 54.0 ± 4.7 | | |
| Exp. B | Peak thrombin | | Peak thrombin |
| Compound | (mean ± | | (mean ± |
| concentration | SD in nM) | | SD in nM) |
| (nM) | for ACE910 | | for mAb4-7762 |
| 0 | 8.5 ± 1.2 | | 8.5 ± 1.2 |
| 0.3 | 7.7 ± 1.0 | | 8.5 ± 1.1 |
| 1 | 7.8 ± 1.0 | | 8.9 ± 0.8 |
| 3 | 8.3 ± 1.2 | | 9.1 ± 1.2 |
| 10 | 9.8 ± 2.2 | | 10.6 ± 0.9 |
| 30 | 9.6 ± 1.0 | | 16.4 ± 0.5 |
| 100 | 13.6 ± 1.1 | | 31.8 ± 7.0 |
| 300 | 19.5 ± 4.7 | | 38.1 ± 0.8 |
| 900 | 39.0 ± 3.1 | | |
| Exp. C | Peak thrombin | | Peak thrombin |
| Compound | (mean ± | | (mean ± |
| concentration | SD in nM) | | SD in nM) |
| (nM) | for ACE910 | | for mAb4-7789 |
| 0 | 7.4 ± 0.1 | | 7.4 ± 0.1 |
| 0.3 | 6.6 ± 0.6 | | 7.9 ± 0.5 |
| 1 | 8.6 ± 2.1 | | 8.0 ± 1.1 |
| 3 | 7.1 ± 0.5 | | 8.6 ± 0.5 |
| 10 | 9.2 ± 2.2 | | 10.6 ± 0.8 |
| 30 | 9.5 ± 0.9 | | 18.6 ± 4.5 |
| 100 | 12.1 ± 1.5 | | 28.2 ± 1.4 |
| 300 | 20.7 ± 7.9 | | 31.5 ± 1.9 |
| 900 | 34.5 ± 3.4 | | 19.5 ± 1.2 |
| Exp. D | Peak thrombin | Peak thrombin | Peak thrombin |
| Compound | (mean ± | (mean ± | (mean ± |
| concentration | SD in nM) | SD in nM) | SD in nM) |
| (nM) | for ACE910 | for mAb5-0057 | for 51409 |
| 0 | 20.7 ± 7.8 | 20.7 ± 7.8 | 20.7 ± 7.8 |
| 0.3 | 26.6 ± 13.2 | 21.7 ± 7.9 | 18.3 ± 7.1 |
| 1 | 21.3 ± 8.9 | 24.5 ± 8.5 | 20.1 ± 8.7 |
| 3 | 23.4 ± 9.9 | 28.7 ± 1.8 | 23.2 ± 10.1 |
| 10 | 25.6 ± 8.6 | 31.2 ± 11.3 | 22.6 ± 5.5 |
| 30 | 26.4 ± 9.3 | 42.9 ± 13.9 | 32.2 ± 12.7 |
| 100 | 32.0 ± 9.9 | 82.6 ± 23.4 | 68.5 24.5 |
| 300 | 46.1 ± 12.3 | 112.3 ± 29.0 | 99.5 27.3 |
| 900 | 71.6 ± 18.4 | 119.7 ± 26.7 | 104.0 30.2 |

TABLE 16
Thrombin generation test (TGT) of the bispecific
antibodies mAb5-0057, mAb5-1409 and ACE910
Thrombin generation test (TGT) of the bispecific
antibodies mAb5-0057, mAb5-1409 and ACE910 in human
tissue factor activated haemophilia A platelet-rich
plasma (PRP). Mean peak thrombin generation ±
standard deviation at each of the tested compound
concentrations from four independent experiments in HA-PRP.

| | Peak thrombin | Peak thrombin | Peak thrombin |
|---|---|---|---|
| Compound | (mean ± | (mean ± | (mean ± |
| concentration | SD in nM) | SD in nM) | SD in nM) |
| (nM) | for ACE910 | for mAb5-0057 | for mAb5-1409 |
| 0 | 15.5 ± 6.6 | 15.5 ± 6.6 | 15.5 ± 6.6 |
| 0.3 | 17.2 ± 7.7 | 16.1 ± 9.2 | 17.2 ± 6.6 |
| 1 | 15.7 ± 5.3 | 19.9 ± 11.4 | 17.4 ± 5.6 |
| 3 | 17.5 ± 6.4 | 21.8 ± 6.5 | 23.7 ± 8.7 |
| 10 | 17.6 ± 6.4 | 36.4 ± 4.9 | 35.6 ± 4.5 |
| 30 | 22.1 ± 6.5 | 65.0 ± 7.1 | 58.8 ± 5.2 |
| 100 | 34.9 ± 9.9 | 87.4 ± 25.3 | 86.0 ± 10.5 |
| 300 | 60.3 ± 16.1 | 100.4 ± 17.6 | 98.6 ± 18.8 |
| 900 | 80.7 ± 11.0 | 105.3 ± 23.8 | 105.3 ± 21.9 |

### Example 18: Activity of Monovalent One-Armed (OA) Anti-FIX/FIXa Antibodies in a Thrombin Generation Test (TGT) in Human Haemophilia a Platelet-Poor Plasma

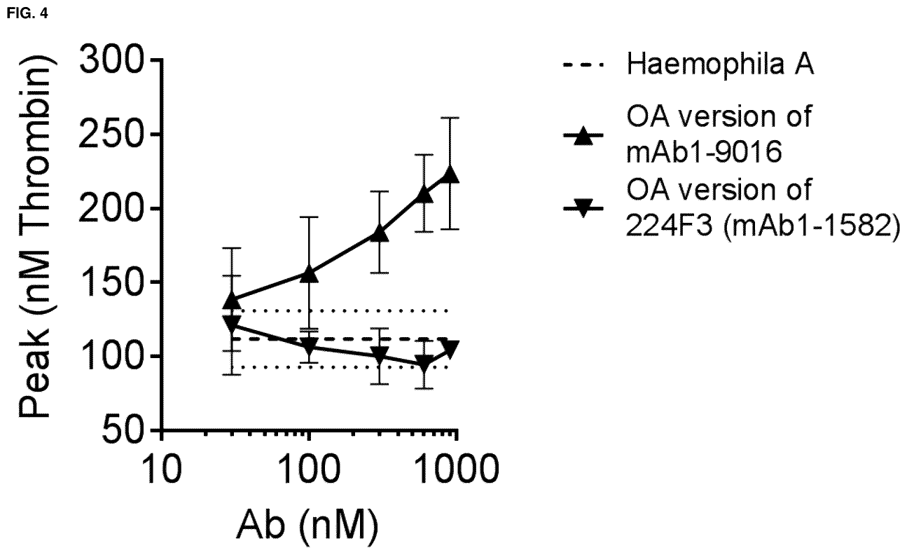

The procoagulant activity of the monovalent one-armed (OA) version of mAb1-9016 was determined based on its ability to promote thrombin generation in the presence of a procoagulant phospholipid membrane according to the principles described by Hemker et al. (2002) Pathophysiol Haemost Thromb, 32:249-253. The one-armed version of the 224F3 antibody (mAb1-1582) was included for comparison. Each antibody (test compound) was tested in a thrombin generation test (TGT) in haemophilia A (HA) patient pooled platelet-poor plasma (HA-PPP) (George King Bio-Medical Inc, KS, USA) by standard calibrated automated thrombography using a 96-well plate fluorometer (Fluoroscan Ascent FL, Thermolabsystems, Helsinki, Finland). Reaction mixtures contained 70 μl HA-PPP, 10 μl test compound (diluted in 20 mM HEPES, 140 mM NaCl, pH 7.4, 2% BSA), 20 μl PRP reagents (synthetic phospholipids, Thrombinoscope BV, Maastricht, the Netherlands) containing activated human plasma-derived factor XI (hFXIa, 8.3 mU/mL final) (Enzyme Research Laboratories, IN, USA) or Thrombin Calibrator (Thrombinoscope BV), and 20 μl of a mixture containing the fluorescent labelled thrombin substrate Z-Gly-Gly-Arg-AMC (3 mM) and CaCl₂ (90 mM) (Thrombinoscope BV). TGT was performed at five concentrations of test compound (30, 100, 300, 600 and 900 nM, final plasma concentration) or added buffer (20 mM HEPES, 140 mM NaCl, pH 7.4, 2% BSA) only (representing HA control). The concentration range was tested in two independent experiments in HA-PPP from the same stock. The TGT was allowed to proceed for a total of 90 minutes and the TGT parameter Peak Thrombin Height (nM) was analysed by Thrombinoscope software (Thrombinoscope BV). FIG. 4 and Table 17 shows/lists the measured peak thrombin generation rates for each monovalent one-armed antibody at the concentrations tested. The data show that the OA antibody version of mAb1-9016 increases the peak thrombin formation above the level observed in the absence of antibody, i.e. exhibit procoagulant activity. In addition, thrombin generation induced by the OA antibody version of mAb1-9016 is higher than that observed for the monovalent OA version of the 224F3 antibody (mAb1-1582).

TABLE 17
Measured peak thrombin generation rates
for each monovalent one-armed antibody at the
concentrations tested. Listed is the average
peak thrombin ± standard deviation of
two independent experiments in HA-PPP

| | Peak thrombin | Peak thrombin |
|---|---|---|
| | (mean ± | (mean ± |
| Compound | SD in nM) | SD in nM) |
| concentration | for OA of 224F3 | for OA of |
| (nM) | (mAb1-1582) | mAb1-9016 |
| 0 | 111.6 ± 19.0 | 111.6 ± 19 |
| 30 | 121.0 ± 33.5 | 138.5 ± 34.9 |
| 100 | 106.2 ± 10.7 | 156.4 ± 37.8 |
| 300 | 100.1 ± 18.9 | 184.0 ± 27.7 |
| 600 | 94.4 ± 16.1 | 210.2 ± 26.1 |
| 900 | 104.0 ± 2.7 | 223.5 ± 37.7 |

### Example 19: Binding Affinities Determined by Isothermal Titration Calorimetry (ITC)

Binding affinities for anti-FIX/FIXa and anti-FX/FXa antibodies binding to FIX/FIXa and FX/FXa, respectively, are measured by isothermal titration calorimetry (ITC) by using a PEAQ-ITC calorimeter (Malvern, UK). The experiments are conducted at 37° C. and pH 7.4 using 25 mM Tris, 150 mM NaCl, 5 mM CaCl₂ (Tris-buffer). The sample cell (200 μl) contains either FIX, FIXa, FX or FXa and anti-FIX/FIXa and anti-FX/FXa antibodies are injected via the syringe. All proteins are extensively dialyzed in Tris-buffer prior to measurements to secure matched buffer conditions. A thermal equilibration step was followed by a 60-s delay and subsequently an initial 0.2-μl injection of antibody, followed by 14 injections of 2.5 μl of antibody at an interval of 120 s. The stirring speed is maintained at 750 rpm, and the reference power is kept constant at 5-10 μcal/s. The heat associated with each injection of antibody is integrated and plotted against the molar ratio of ligand to macromolecule. The resulting isotherm is fitted to a one-site binding model to obtain the affinity (KD), stoichiometry (n), and enthalpy of interaction (ΔH) using the software provided by the manufacturer. Experiments were performed in duplicate or triplicate.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A bispecific antibody or antigen-binding fragment thereof capable of stimulating the enzymatic activity of FIXa towards FX comprising a first antigen-binding site capable of binding to FIX (SEQ ID NO:1) and/or the activated form thereof (FIXa), and a second antigen-binding site capable of binding to FX (SEQ ID NO:2) and/or the activated form thereof (FXa), wherein the first antigen-binding site is capable of binding an epitope comprising amino acid residue R338 of FIX/FIXa, and
the second antigen-binding site is capable of binding in the EGF-2 domain and/or catalytic subunit of FX/FXa and binding an epitope comprising amino acid residues Y230, D423 and K427 of FX/FXa.

2. The antibody or antigen-binding fragment thereof according to claim 1, wherein the first antigen-binding site is capable of binding an epitope comprising amino acid residues R338 and K341 of FIX/FIXa.

3. The antibody or antigen-binding fragment thereof according to claim 2, wherein the first antigen-binding site is capable of binding an epitope comprising amino acid residues L337, R338, S339, T340, K341 and T343 of FIX/FIXa.

4. The antibody or antigen-binding fragment thereof according to claim 1, wherein the first antigen-binding site is capable of binding an epitope comprising amino acid residues D332, R333, L337 and R338 of FIX/FIXa.

5. The antibody or antigen-binding fragment thereof according to claim 1, wherein the first antigen-binding site is capable of binding an epitope comprising amino acid residues K301, D332, R333, A334, T335, R338 and N346 of FIX/FIXa.

6. The antibody or antigen-binding fragment thereof according to claim 1, wherein the second antigen-binding site is capable of binding an epitope comprising amino acid residues R113, Y230, K420, D423, R424 and K427 of FX/FXa.

7. The antibody or antigen-binding fragment thereof according to claim 6, wherein the second antigen-binding site is capable of binding an epitope comprising amino acid residues H101, E103, R113, T116, L117, A118, T127, S227, E228, F229, Y230, E266, R287, P304, E305, L333, L419, K420, D423, R424, M426, K427 and T428 of FX/FXa.

8. A bispecific antibody or antigen-binding fragment thereof capable of stimulating the enzymatic activity of FIXa towards FX comprising a first antigen-binding site capable of binding to FIX (SEQ ID NO:1) and/or the activated form thereof (FIXa), and a second antigen-binding site capable of binding to FX (SEQ ID NO:2) and/or the activated form thereof (FXa), wherein said bispecific antibody or antigen-binding fragment thereof comprises
a) a heavy chain variable domain which is at least 96% identical to the sequence identified by SEQ ID NO:177 and a light chain variable domain which is at least 96% identical to the sequence identified by SEQ ID NO:178, which are capable of binding to FIX and/or the activated form thereof (FIXa), and
b) a heavy chain variable domain at least 96% identical to the sequence identified by SEQ ID NO:21 and a light chain variable domain at least 96% identical to the sequence identified by SEQ ID NO:22, which are capable of binding to FX and/or the activated form thereof (FXa).

9. A pharmaceutical composition comprising the antibody or antigen-binding fragment thereof according to claim 1.

10. A pharmaceutical composition comprising the antibody or antigen-binding fragment thereof according to claim 8.

11. A method of treating a subject suffering from a coagulopathy or blood coagulation disorder, comprising administering to said subject an antibody or antigen-binding fragment thereof according to claim 1.

12. The method of claim 11, wherein said coagulopathy or blood coagulation disorder is haemophilia A with or without inhibitors.

13. A method of treating a subject suffering from a coagulopathy or blood coagulation disorder, comprising administering to said subject an antibody or antigen-binding fragment thereof according to claim 8.

14. The method of claim 13, wherein said coagulopathy or blood coagulation disorder is haemophilia A with or without inhibitors.


SEQUENCE LISTING (202 sequences)

SEQ ID NO: 1 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - protein, 448 aa, Homo sapiens [sequence omitted]

SEQ ID NO: 3 - protein, 122 aa, Homo sapiens
EVQLVESGGGLVQPGRSLKLSCAASGFTFDDYAMHWVRQVPGKGLEWVSGISWRGDIIGYVDSVKGRFTISRDNAKNSLYLQLNSLRAEDTALYYCVKSYGSGSFYNAFDSWGQGTLVTVSS

SEQ ID NO: 4 - protein, 107 aa, Homo sapiens
DIQMTQSPSTLSASAGDRVTITCRASQSISSWLAWYQQKPGKAPKFLIYKASRLERGVPSRFSGSGSGTEFSLTISSLQPDDFATYYCLEYSSYIRTFGQGTKVEIK

SEQ ID NO: 5 - protein, 122 aa, Homo sapiens
EVQLVESGGGLVQPGRSLKLSCAASGFTFDDYAMHWVRQVPGKGLEWVSGISWNGDNIGYVDSVKGRFTISRDNAKNSLYLQLNSLRAEDTALYYCVKSYGSGSFYNAFDSWGQGTLVTVSS

SEQ ID NO: 6 - protein, 107 aa, Homo sapiens; identical to SEQ ID NO: 4

SEQ ID NO: 7 - protein, 120 aa, Homo sapiens
EVQLVESGGGLVQPGRSLRLSCAASGFTFGDYAMHWVRQAPGKGLEWVSGISWVSGSIGYARSVKGRFTISRDNAKNSLYLQMNSLRAEDTALYYCAKGQYDEDAGMDVWGQGTTVTVSS

SEQ ID NO: 8 - protein, 106 aa, Homo sapiens
DIQMTQSPSSLSASVGDRVTITCRASQSIRSYLNWYQQKPGKAPKLLIYAASSLQSGVPSRFSGSGSGTDFTLTISSLQPEDFATYYCQQGYPTWTFGGGTKVEIK

SEQ ID NO: 9 - protein, 120 aa, Homo sapiens
EVQLVESGGGLVQPGRSLRLSCAASGFTFGDYAMHWVRQAPGKGLEWVSGISWNSGSIGYAKSVKGRFTISRDNAKNSLYLQMNSLRAEDTALYYCAKGQYDEDAGMDVWGQGTTVTVSS

SEQ ID NO: 10 - protein, 106 aa, Homo sapiens
DIQMTQSPSSLSASVGDRVTITCRASQWISSYLNWYQQKPGKAPKLLIYAASSLPSGVPSRFSGSGSGTDFTLTISSLQPEDFATYYCQQGYPTWTFGGGTKVEIK

SEQ ID NO: 11 - protein, 119 aa, Homo sapiens
EVQLVQSGAEVKKPGESLRISCKGSGYSFTPYWIGWVRQMPGKGLEWMGMIDPSDSYTDYGPSFRGHVTISADKSISTAYLQWSSLKASDTAMYYCARVRIREGAFDIWGQGTMVTVSS

SEQ ID NO: 12 - protein, 112 aa, Homo sapiens
DIVMTQSPLSLPVTPGEPASISCRSSQRLLHSTGYNYLNWYLQKPGQSPQLLIYLGSRRASGVPDRFSGSGSGTDFTLKISRVEAEDVGVYYCMQALHIPYTFGQGTKLEIK

SEQ ID NO: 13 - protein, 119 aa, Homo sapiens
EVQLVQSGAEVKKPGESLRISCKGSGYRFTSYWIGWVRQMPGKGLEWMGMIDPTDSYTDYGPSFRGHVTISADKSISTAYLQWSSLKASDTAMYYCARVRIMSGAFDIWGQGTMVTVSS

SEQ ID NO: 14 - protein, 112 aa, Homo sapiens
DIVMTQSPLSLPVTPGEPASISCRSSQSLLHSTWYNYLNWYLQKPGQSPQLLIYLGSNRASGVPDRFSGSGSGTDFTLKISRVEAEDVGVYYCMQVLHIPYTFGQGTKLEIK

SEQ ID NO: 15 - protein, 122 aa, Homo sapiens; identical to SEQ ID NO: 5

SEQ ID NO: 16 - protein, 107 aa, Homo sapiens
DIQMTQSPSTLSASAGDRVTITCRASQSISSWLAWYQQKPGKAPKFLIYKASSLESGVPSRFSGSGSGTEFSLTISSLQPDDFATYYCLEYSSYIRTFGQGTKVEIK

SEQ ID NO: 17 - protein, 120 aa, Homo sapiens
EVQLVESGGGLVQPGRSLRLSCAASGFTFGDYAMHWVRQAPGKGLEWVSGISWNSGSIGYADSVKGRFTISRDNAKNSLYLQMNSLRAEDTALYYCAKGQYDEDAGMDVWGQGTTVTVSS

SEQ ID NO: 18 - protein, 106 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQGYPTWTFGGGTKVEIK

SEQ ID NO: 19 - protein, 119 aa, Homo sapiens
EVQLVQSGAEVKKPGESLRISCKGSGYSFTSYWIGWVRQMPGKGLEWMGMIDPSDSYTDYGPSFRGHVTISADKSISTAYLQWSSLKASDTAMYYCARVRIMEGAFDIWGQGTMVTVSS

SEQ ID NO: 20 - protein, 112 aa, Homo sapiens
DIVMTQSPLSLPVTPGEPASISCRSSQSLLHSTGYNYLNWYLQKPGQSPQLLIYLGSNRASGVPDRFSGSGSGTDFTLKISRVEAEDVGVYYCMQALHIPYTFGQGTKLEIK

SEQ ID NO: 21 - protein, 120 aa, Homo sapiens
EVQLVQSGAEVKKPGESLRISCKGSGYSFTTSWIVWVRQMPGKGLEWMGMIDPSDSFTSYSPSFQGHVTISADKSISTAYLQWSSLKASDTAMYYCARLHYYHSEEFDVWGQGTLVTVSS

SEQ ID NO: 22 - protein, 109 aa, Homo sapiens
EIVLTQSPGTLSLSPGERATLSCRASQSVSSSYLAWYQQKPGQAPRLLIYGASSRARGIPDRFSGSGSGTDFTLTISRLEPEDFAVYYCQQFGSSRLFTFGQGTKLEIK

SEQ ID NO: 23 - protein, 120 aa, Homo sapiens
EVQLLESGGGLVQPGGSLRLSCAASGFTFSDYAVSWVRQAPGKGLEWVSTLYTGGSTHYADSVKGRFTISRDNSKNTLYLQMNSLRAEDTAVYYCAATWELLSIVAFDIWGQGTMVTVSS

SEQ ID NO: 24 - protein, 112 aa, Homo sapiens
DIVMTQSPLSLPVTPGEPASISCRSSQSLLHSDGYTYLDWYLQKPGQSPQLLIYLGSNRASGVPDRFSGSGSGTDFTLKISRVEAEDVGVYYCMQVLHTPRTFGQGTKLEIK

SEQ ID NO: 25 - protein, 121 aa, Homo sapiens
EVQLLESGGGLVQPGGSLRLSCAASGFTFSNNYMSWVRQAPGKGLEWVSTIGGSGGRTYYADSVKGRFTISRDNSKNTLYLQMNSLRAEDTAVYYCARGGSWGFGWYFDLWGRGTLVTVSS

SEQ ID NO: 26 - protein, 109 aa, Homo sapiens
EIVLTQSPGTLSLSPGERATLSCRASQSVGGSYLAWYQQKPGQAPRLLIYDTSSRATGIPDRFSGSGSGTDFTLTISRLEPEDFAVYYCQQQYGTSPGTFGQGTKLEIK

SEQ ID NO: 27 - protein, 122 aa, Homo sapiens
EVQLVQSGAEVKKPGASVKVSCKASGYTFTSYAIHWVRQAPGQGLEWMGWISPHNGNTHYAQKFQGRVTMTTDTSTSTAYMELRSLRSDDTAVYYCARGSLWFGELLPFDYWGQGTLVTVSS

SEQ ID NO: 28 - protein, 108 aa, Homo sapiens
EIVLTQSPGTLSLSPGERATLSCRASQRVSSSFLAWYQQKPGQAPRLLIYAVSSRATGIPDRFSGSGSGTDFTLTISRLEPEDFAVYYCQHYGSSPYTFGQGTKLEIK

SEQ ID NO: 29 - protein, 120 aa, Homo sapiens
EVQLVQSGAEVKKPGESLRISCKGSGYSFTKYWIGWVRQMPGKGLEWMGMIDPTDSYTTYSPSLHGHVTISADKSISTAYLQWSSLKASDTAMYYCARRQLWLRDAFDIWGQGTMVTVSS

SEQ ID NO: 30 - protein, 107 aa, Homo sapiens
EIVLTQSPGTLSLSPGERATLSCRASQNVGSNLAWYQQKPGQAPRLLIYDASSRAAGIPDRFSGSGSGTDFTLTISRLEPEDFAVYYCQQYDYWPATFGGGTKVEIK

SEQ ID NO: 31 - protein, 121 aa, Homo sapiens
EVQLVQSGAEVKKPGESLRISCKGSGYSFTSYWIVWVRQMPGKGLEWMGMIDPSDSYTDYSPSFEGHVTISADKSISTAYLQWSSLKASDTAMYYCTRSQRRGYLDAFDIWGQGTMVTVSS

SEQ ID NO: 32 - protein, 108 aa, Homo sapiens
EIVLTQSPGTLSLSPGERATLSCRASQGIGTDLAWYQQNPGQAPRLLIYAASARATGIPDRFSGSGSGTDFTLTISRLEPEDFAVYYCQQYNNWPLYTFGQGTKLEIK

SEQ ID NO: 33 - protein, 118 aa, Homo sapiens
EVQLVQSGAEVKKPGESLRISCKGSGYSFTSYWIGWVRQMPGKGLEWMGMIDPSDSYTTYSPSFEGHVTISADKSISTAYLQWSSLKASDTAMYYCARPSFTDHFDYWGQGTLVTVSS

SEQ ID NO: 34 - protein, 106 aa, Homo sapiens
EIVLTQSPGTLSLSPGERATLSCRASQSVSSNLAWYQQKPGQAPRLLIYASARAAGIPDRFSGSGSGTDFTLTISRLEPEDFAVYYCQQYDNWPATFGGGTKVEIK

SEQ ID NO: 35 - protein, 121 aa, Homo sapiens
EVQLLESGGGLVQPGGSLRLSCAASGFTFSDYAISWVRQAPGKGLEWVSAISDSGGSTYYADSVKGRFTISRDNSKNTLYLQMNSLRAEDTATYYCAHSEDYYDSSGFDYWGQGTLVTVSS

SEQ ID NO: 36 - protein, 108 aa, Homo sapiens
EIVLTQSPGTLSLSPGERATLSCRASQSISSNLAWYQQKPGQAPRLLIYDASARAAGIPDRFSGSGSGTDFTLTISRLEPEDFAVYYCQQYDNWPPVTFGQGTKLEIK

SEQ ID NO: 37 - protein, 126 aa, Homo sapiens
EVQLVESGGGLVQPGGSLRLSCAASGFTFSTYALTWVRQAPGKGLDWVSTISGSGGNTYYADSVKGRFTISRDNSRNTLSLQMNSLRADETAVYYCAKGGFASSSGRYYYYAMDVWGQGTTVTVSS

SEQ ID NO: 38 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 39 - protein, 121 aa, Homo sapiens
QAQLVESGGGVVQPGRSLRLSCAASGFTFSSYGMHWVRQAPGKGLEWVAVTSYDGSNEYYADSVKGRFTISRDNSKNTLYLQMNSLRAEDTAVYYCAKEQWLVPDDAFDIWGQGTMVTVSS

SEQ ID NO: 40 - protein, 113 aa, Homo sapiens
DIVMTQSPDSLAVSLGERATINCKSSQSVLYSSSNKNFLAWYQQKPGQPPNLIIYWASNRESGVPDRFSGSGSGTDFTLTISSLQAEDVAVYYCHQYYNTPPTFGQGTKVEIK

SEQ ID NO: 41 - protein, 123 aa, Homo sapiens
QVQLVQSGAEVKKPGASVKVSCKASGYTLRSYGISWLRQAPGQGLEWMGWISAYNGDTNYAQKFQGRVTLTTEKSTSTAYMELRSLRSDDMAVYYCAKEDISSRWSPDTFDIWGQGTLVTVSS

SEQ ID NO: 42 - protein, 107 aa, Homo sapiens
DIQMTQSPSTLSASVGDRVTITCRASQSISSWLAWYQQKPGKAPKLLIYKASSLETGVPSRFSGSGYGTEFTLTISSLQSDDFATYYCQQYNHYSRTFGQGTKLEIK

SEQ ID NO: 43 - protein, 124 aa, Homo sapiens
EVQLVESGGGLVQPGRSLRLSCAASGFTFDDNAMHWVRQAPGKGLEWVSGISWNSGSIGYADSVKGRFTISRDNAKNSLYLQMNSLRAEDTALYYCAKDWAMVRGVITNAFDIWGQGTMVTVSS

SEQ ID NO: 44 - protein, 106 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQFNRYSTFGQGTKVEIK

SEQ ID NO: 45 - protein, 120 aa, Homo sapiens
QLQLQESGPGLVKPSETLSLTCTVSGGSISSNYYYWGWIRQTPGKRLEWIGSIYYSGSTYYNPSLKSRVTISIDTSKNQFSLTLSFVTAADTAVYYCAREAERHDSFDIWGQGTMVTVSS

SEQ ID NO: 46 - protein, 107 aa, Homo sapiens
EIVMTQSPVTLSVSPGERATLSCRASQNVYNNLAWYQQKPGQAPRLLIYGASTRATGFPGRFSGSGSGTEFTLTISSLQSEDFAVYYCQQYNVWPFTFGPGTKVDIK

SEQ ID NO: 47 - protein, 120 aa, Homo sapiens
QLQLQESGPGLVKPSETLSLTCTVSGGSISSSYYYWGWIRQTPGKGLEWIGSIYYSGSTYYNPSLKSRVTISIDTSKNQFSLTLSFVTAADTAVYYCAREAERHDSFDLWGQGTMVTVSS

SEQ ID NO: 48 - protein, 107 aa, Homo sapiens
EIVMTQSPVTLSVSPGERATLSCRASQNVYNNLAWYQQKPGQAPRLLIYGASTRATGIPGRFSGSGSGTEFTLTISSLQSEDFAVYYCQQYNVWPFTFGPGTKVDIK

SEQ ID NO: 49 - protein, 116 aa, Homo sapiens
QEQLVESGGDVVQPGKSLRLSCTTSGFTFSFYGIHWVRQAPGKGLEWVTSISYDGTNKYYADSVKGRFTISRDNSKGTLYLQMDSLRPEDTAVYYCVKDSRGMAVWGQGTTVTVSS

SEQ ID NO: 50 - protein, 112 aa, Homo sapiens
DVLMTQSPLFLPVTFGQPASISCRSSRSLVYSDGNTYVNWFHQRPGQSPRRLIYKVSNRDSGVPDRFSGSGSGTDFTLKISRVEAEDVGVYYCMQGTHWPPTFGQGTKVEIK

SEQ ID NO: 51 - protein, 120 aa, Homo sapiens
QLQLQESGPGLVKPSETLSLTCTVSGGSISSSYYYWGWIRQTPGKGLEWIGSIYYSGSTYYNPSLKSRVTISVDTSKNHFSLNLSFVTAADTAVYYCAREAERHDTFDIWGQGTMVTVSS

SEQ ID NO: 52 - protein, 107 aa, Homo sapiens
EIVMTQSPVTLSVSPGERATLSCRASQNISNNLAWYQQKPGQAPRLLIYGASTRATGIPGRFSGSGSGTEFTLTISSLQSEDFAVYYCQQYNNWPFTFGPGTKVDIK

SEQ ID NO: 53 - protein, 119 aa, Homo sapiens
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHRLGSYEFDYWGQGTLVTVSS

SEQ ID NO: 54 - protein, 112 aa, Homo sapiens
DIVMTQSPLSLPVTPGEPASISCRSSQSLLHSNGYSYLDWYLQRPGQSPQLLIYLGSNRASGVPDRFSGSGSGTDFTLKISRVEAEDVGVYYCMQALQTPLTFGGGTKVEIK

SEQ ID NO: 55 - protein, 117 aa, Homo sapiens
QVQLVQSGAEVKKPGASVKVSCKASGYTFTGYYIHWVRQAPGQGLEWMGWINPNSGSTNYAQKFQGWVTMTRDTSISTAYMELSRLRSDATAVYYCARGYYDIFDYWGQGTLVTVSS

SEQ ID NO: 56 - protein, 107 aa, Homo sapiens
EIVLTQSPDFQSVTPKEKVTITCRASQTIGNSLHWYQQKPDQSPKLLIEYASQSISGVPSRFSGSGSGTDFTLTIISLEAEDAAAYYCHQSSRLPWTFGQGTKVEIK

SEQ ID NO: 57 - protein, 124 aa, Homo sapiens
EVQLVQSGAEVKKPGESLKISCKGFGYRFTDYWIGWIRQMPGKGLEWMGIIYPDDSDTRYSPSFQGQVTISADRSVKTAYLQWSSLKASDTAMYYCARHGIIGTRDYYYGMDVWGQGTTVTVSS

SEQ ID NO: 58 - protein, 107 aa, Homo sapiens
DIQMTQSPSSLSASVGDRVTITCQASQDINNYLDWYQQKPGKAPKLLIYDASTLITGVPSRFSGSGSGTYFTFTISSLQPDDIATYYCQHYDNLPFTFGPGTKLEIK

SEQ ID NO: 59 - protein, 124 aa, Homo sapiens
EVQLVESGGGLVQPGRSLRLSCAASGLTFDDYAMHWVRQAPGKGLEWVSGISWNGGSKEYAASVKGRFTISRDNAKNFLYLQMNSLRVEDTALYYCAKDWAMVRGVITNVFDIWGQGTMVTVSS

SEQ ID NO: 60 - protein, 106 aa, Homo sapiens
AIQMTQSPSTLSASVGDRVTITCRASQTISSWLAWYQQKPGKAPKLLIYKASSLESGVPSRFSGSGSGTEFTLTISSLQPDDFATYYCQQYSSYSTFGQGTKVEIK

SEQ ID NO: 61 - protein, 119 aa, Homo sapiens
QEQLVESGGGVVQPGRSLRLSCAASGFNFNTYAMHWVRQAPGKGLEWVTVISFDSSNKYYADSVKGRFTISRDNSKNTLYLEMNSLKPEDTAIYYCVKGRSYYWYFDLWGRGTLVTVSS

SEQ ID NO: 62 - protein, 112 aa, Homo sapiens
NIIMTQTPLSLPVSLGQPASISCRSSQSLVHSDGNTYLTWLQQRPGQPPRLLIYKISKRFSGVPDRFSGSGAGTDFTLKISRVEAEDVGIYYCMQATHFPHTFGGGTKVEIK

SEQ ID NO: 63 - protein, 121 aa, Homo sapiens
EVQLVESGGGLVQPGGSLRLSCTASGFTFSSSAMSWVRQAPGKGLYWVSAISGNGDNTYYADSVKGRFTISRDNSKNTLFLQMNSLRAEDTALYYCAEEGIVVAHDAFEIWGQGTMVTVSS

SEQ ID NO: 64 - protein, 113 aa, Homo sapiens
DIVMTQSPDSLAVSLGERATINCKSSQSVLYSSNNKNYLAWYQQKPGQPPKLLIYWASIRESGVPDRISGSGSGTDFTLTISSLQAEDVAVYYCQQYYGTPITFGQGTRLEIK

SEQ ID NO: 65 - protein, 109 aa, Oryctolagus cuniculus
QERLKESGGRLVTPGTPLTLTCTAGGSDISVYGIHWVRQAPGKGLEWIGWIGAGGHAYYARWAVGRFTISRTSTTVDLKMTSPTTEDTATYFCASAGLWGPGTLVTVSS

SEQ ID NO: 66 - protein, 112 aa, Oryctolagus cuniculus
QVLTQTESPVSAAIGGTVTINCQSSQSVVNDNRLSWYQQKPGQPPKRLIYDASKLASGVPSRFKGSGSGTQFTLTISDVQCDDAATYYCLGGYDCSLNDCNGFGGGTEVVVK

SEQ ID NO: 67 - protein, 119 aa, Oryctolagus cuniculus
QSVEESGGRLVTPGTPLTLTCTVSGIDLSTYTMGWVRQAPGKGLEYIGIIASSGHAYYASWAKGRFTISKTSSTAVDLKMTSLTTEDTATYFCVRALYSGGGYWPGGFWGPGTLVTVSS

SEQ ID NO: 68 - protein, 111 aa, Oryctolagus cuniculus
IVMTQTPSSKSVPVGDTVTINCQASKSVYSNNRLAWYQQKPGQPPKLLIYSASTLASGVPSRFRGSGSGTQFTLTISDVVCDDAATYYCAGYEESIDDDTAFGGGTEVVVK

SEQ ID NO: 69 - protein, 122 aa, Homo sapiens
EVQLVESGGGLVQPGRSLKLTCAASGFTFDDYAMHWVRQVPGKGLEWVSGISWNGDNIGYVESVKGRFTISRDNDKNSLHLELNSLRPEDTALYFCAKSYGSGSYYNAFDSWGQGTLVTVSS

SEQ ID NO: 70 - protein, 107 aa, Homo sapiens; identical to SEQ ID NO: 16

SEQ ID NO: 71 - protein, 122 aa, Homo sapiens
EVQLVESGGGLVQPGRSLRLSCAASRFTFDDYAMHWVRQVPGKGLEWVSGISWNSDNIGYADSVKGRFTISRDNAKNSLYLQMNSLRAEDTALYYCAKAFGSGSYYNGFDYWGQGTLVTVSS

SEQ ID NO: 72 - protein, 107 aa, Homo sapiens
DIQMTQSPSTLSASAGDRVTISCRASQSISSWLAWYQQKPGKAPKLLIYKASSLESGVPSRFSGSGSGTEFTLTISSLQPDDFATYYCQEYNSYIRTFGQGTKVEIK

SEQ ID NO: 73 - protein, 122 aa, Homo sapiens
EVQLVESGGGLVQPGRSLRLSCAASGFIFDDYAMHWVRQVPGKGLEWVSGISWNSDNIGYVDSVKGRFTISRDNAKNSLYLQMNSLRIEDTALYYCAKAFGSGSYYNGFDYWGQGALVTVSS

SEQ ID NO: 74 - protein, 107 aa, Homo sapiens; identical to SEQ ID NO: 72

SEQ ID NO: 75 - protein, 121 aa, Mus musculus
QVQLQQSGAELVRPGSSVKISCKASAYEFSKHWMNWVKQRPGQGLEWVGQIYPGDGDTNYNRKFKGKATLTADKSSSTAYMQLSSLTSEDSAVYFCARYGYDEEYYGMDYWGQGTSVTVSS

SEQ ID NO: 76 - protein, 107 aa, Mus musculus
DIQMTQSPASLSASVGEAVTITCRVNENIDSYLAWYQQKQGKSPQLLVYAATLLADGVPPRFSGSGSGTQFSLKINSLQSEDVARYFCQHYYSTPWTFGGGTKLEIK

SEQ ID NO: 77 - protein, 121 aa, Mus musculus
QVQLQQSGAELVRPGSSVKISCKASGYAFSKHWMNWVKQRPGQGLEWIGQIYPGDGDINYNGKFKGKATLTADKSSSTAYMQLSSLTSEDSAVYFCARYGYNEEYYGLDYWGQGTSVTVSS

SEQ ID NO: 78 - protein, 107 aa, Mus musculus
DIQMTQSPASLSASVGETVTITCRISENIDSYLAWYQQKQGKSPQLLVYAATLLADGVPSRFSGSGSGTQFSLKINSLQSEDVARYFCQHYYSTPWTFGGGTKLEIK

SEQ ID NO: 79 - protein, 117 aa, Oryctolagus cuniculus
QSLAESGGRLVTPGTPLTLTCTAPGFTIGGYHMSWVRQAPGKGLEWIGLINNGGSTAYANWVKGRFTISKTSTTVDLKLTSLTSEGTATYFCARWTGYAGDGYGDIWGQGTLVTVSS

SEQ ID NO: 80 - protein, 111 aa, Oryctolagus cuniculus
ADIVMTQTPASVSAAVGGTVTFKCQASENIYNLLAWYQQKPGQPPKLLIYDAFDLASGVPSRFKGSGSGTQFTLTISDLESADAATYYCQSYYYASSSSYPFGGGTEVVVK

SEQ ID NO: 81 - protein, 122 aa, Homo sapiens; identical to SEQ ID NO: 73

SEQ ID NO: 82 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCGQYRSYSRTFGGGTKVEIK

SEQ ID NO: 83 - protein, 122 aa, Homo sapiens; identical to SEQ ID NO: 5

SEQ ID NO: 84 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCLQYRSYSRTFGGGTKVEIK

SEQ ID NO: 85 - protein, 120 aa, Oryctolagus cuniculus
QSVEESGGRLVTPGTPLTLTCTVSGFSLSAYAVSWVRQAPGKGLEWIGIVSSSDFTYYASWAKGRFTISKTSTTVELKITSPTTEDTATYFCARDLNYDDYENYYGMDPWGPGTLVTVSS

SEQ ID NO: 86 - protein, 113 aa, Oryctolagus cuniculus
DVVMTQTPSSVSAAVGGTVTIKCQSSQSVYNYNLLSWYQQKPGQPPKLLIYGASNLASGVPDRFSGSGSGTQFTLTISGVQCDDAATYYCLGSFDDDVDYENAFGGGTEVVVK

SEQ ID NO: 87 - protein, 119 aa, Oryctolagus cuniculus; identical to SEQ ID NO: 67

SEQ ID NO: 88 - protein, 110 aa, Oryctolagus cuniculus
VMTQTPSSKSVPVGDTVTINCQASKSVYSNNRLAWYQQKPGQPPKLLIYSASTLASGVPSRFRGSGSGTQFTLTISDVVCDDAATYYCAGYEESIDDDTAFGGGTEVVVK

SEQ ID NO: 89 - protein, 119 aa, Homo sapiens; identical to SEQ ID NO: 11

SEQ ID NO: 90 - protein, 112 aa, Homo sapiens; identical to SEQ ID NO: 12

SEQ ID NO: 91 - protein, 119 aa, Homo sapiens
EVQLVQSGAEVKKPGESLRISCKGSGYSFRPYWIGWVRQMPGKGLEWMGMIDPSDSYTDYGPSFRGHVTISADKSISTAYLQWSSLKASDTAMYYCARVRIRAGAFDIWGQGTMVTVSS

SEQ ID NO: 92 - protein, 112 aa, Homo sapiens
DIVMTQSPLSLPVTPGEPASISCRSSQSLLHRTYYNYLNWYLQKPGQSPQLLIYLGKNRASGVPDRFSGSGSGTDFTLKISRVEAEDVGVYYCMQVLHIPYTFGQGTKLEIK

SEQ ID NO: 93 - protein, 123 aa, Homo sapiens
EVQLVESGGGLVKPGGSLRLSCAASGFTFSNAWMNWVRQAPGKGLEWVGRIKRKTDGGTTDYAAPVKGRFTISRNDSKNTLYLQMNSLKTEDTAVYYCTTDGIAVAGPLRVYWGQGTLVTVSS

SEQ ID NO: 94 - protein, 113 aa, Homo sapiens
DIVMTQSPDSLAVSLGERATINCKSSQSVLYSSNNKNYLVWYQQKPGQPPKLLIYWASTRESGVPDRFSGSGSGTDFTLTISSLQAEDVAVYYCQQYYSIPRTFGQGTKVEIK

SEQ ID NO: 95 - protein, 119 aa, Homo sapiens
EVQLVQSGAEVKKPGESLRISCKGSGYSFRPYWIGWVRQMPGKGLEWMGMIDPSDSYTDYGPSFEGHVTISADKSISTAYLQWSSLKASDTAMYYCARVRIRAGAFDIWGQGTMVTVSS

SEQ ID NO: 96 - protein, 112 aa, Homo sapiens
DIVMTQSPLSLPVTPGEPASISCRSSQRLLHSTGYNYLNWYLQKPGQSPQLLIYLGKNRASGVPDRFSGSGSGTDFTLKISRVEAEDVGVYYCMQVLHIPYTFGQGTKLEIK

SEQ ID NO: 97 - protein, 119 aa, Homo sapiens
EVQLVQSGAEVKKPGESLRISCKGSGYSFKPYWIGWVRQMPGKGLEWMGMIDPSDSYTDYGPSFRGHVTISADKSISTAYLQWSSLKASDTAMYYCARVRIRAGAFDIWGQGTMVTVSS

SEQ ID NO: 98 - protein, 112 aa, Homo sapiens
DIVMTQSPLSLPVTPGEPASISCRSSQSLLHRTWYNYLNWYLQKPGQSPQLLIYLGRNRASGVPDRFSGSGSGTDFTLKISRVEAEDVGVYYCMQVLHIPYTFGQGTKLEIK

SEQ ID NO: 99 - protein, 119 aa, Homo sapiens; identical to SEQ ID NO: 91

SEQ ID NO: 100 - protein, 112 aa, Homo sapiens; identical to SEQ ID NO: 98

SEQ ID NO: 101 - protein, 119 aa, Homo sapiens
EVQLVQSGAEVKKPGESLRISCKGSGYSFRPYWIGWVRQMPGKGLEWMGMIDPSDSYTDYGPSFHGHVTISADKSISTAYLQWSSLKASDTAMYYCARVRIMSGAFDIWGQGTMVTVSS

SEQ ID NO: 102 - protein, 112 aa, Homo sapiens
DIVMTQSPLSLPVTPGEPASISCRSSQSLLHSTWYNYLNWYLQKPGQSPQLLIYLGKNRASGVPDRFSGSGSGTDFTLKISRVEAEDVGVYYCMQVLHIPYTFGQGTKLEIK

SEQ ID NO: 103 - protein, 119 aa, Homo sapiens
EVQLVQSGAEVKKPGESLRISCKGSGYRFTSYWIGWVRQMPGKGLEWMGMIDPTDSYTDYGPSFHGHVTISADKSISTAYLQWSSLKASDTAMYYCARMRIMSGAFDIWGQGTMVTVSS

SEQ ID NO: 104 - protein, 112 aa, Homo sapiens
DIVMTQSPLSLPVTPGEPASISCRSSQSLLHSTWYNYLNWYLQKPGQSPQLLIYLGSRRASGVPDRFSGSGSGTDFTLKISRVEAEDVGVYYCMQVLHIPYTFGQGTKLEIK

SEQ ID NO: 105 - protein, 119 aa, Homo sapiens; identical to SEQ ID NO: 95

SEQ ID NO: 106 - protein, 112 aa, Homo sapiens; identical to SEQ ID NO: 98

SEQ ID NO: 107 - protein, 119 aa, Homo sapiens; identical to SEQ ID NO: 95

SEQ ID NO: 108 - protein, 112 aa, Homo sapiens; identical to SEQ ID NO: 102

SEQ ID NO: 109 - protein, 119 aa, Homo sapiens; identical to SEQ ID NO: 91

SEQ ID NO: 110 - protein, 112 aa, Homo sapiens; identical to SEQ ID NO: 102

SEQ ID NO: 111 - protein, 119 aa, Homo sapiens; identical to SEQ ID NO: 103

SEQ ID NO: 112 - protein, 112 aa, Homo sapiens; identical to SEQ ID NO: 102

SEQ ID NO: 113 - protein, 119 aa, Homo sapiens; identical to SEQ ID NO: 95

SEQ ID NO: 114 - protein, 112 aa, Homo sapiens; identical to SEQ ID NO: 104

SEQ ID NO: 115 - protein, 119 aa, Homo sapiens; identical to SEQ ID NO: 91

SEQ ID NO: 116 - protein, 112 aa, Homo sapiens; identical to SEQ ID NO: 104

SEQ ID NO: 117 - protein, 119 aa, Homo sapiens; identical to SEQ ID NO: 95

SEQ ID NO: 118 - protein, 112 aa, Homo sapiens
DIVMTQSPLSLPVTPGEPASISCRSSQSLLHSTWYNYLNWYLQKPGQSPQLLIYLGSNRASGVPDRFSGSGSGTDFTLKISRVEAEDVGVYYCMQALHIPYTFGQGTKLEIK

SEQ ID NO: 119 - protein, 119 aa, Homo sapiens
EVQLVQSGAEVKKPGESLRISCKGSGYRFTPYWIGWVRQMPGKGLEWMGMIDPTDSYTDYGPSFRGHVTISADKSISTAYLQWSSLKASDTAMYYCARVRIRSGAFDIWGQGTMVTVSS

SEQ ID NO: 120 - protein, 112 aa, Homo sapiens; identical to SEQ ID NO: 118

SEQ ID NO: 121 - protein, 122 aa, Homo sapiens
EVQLVESGGGLVQPGRSLKLSCAASGFRFDDYAMHWVRQAPGKGLEWVSGISWRGAQIGYADSVKGRFTISRDNAKNSLYLQLNSLRAEDTALYYCAKSYGSGSFYNAFDSWGQGTLVTVSS

SEQ ID NO: 122 - protein, 107 aa, Homo sapiens
DIQMTQSPSTLSASVGDRVTITCRASQKISSWLAWYQQKPGKAPKFLIYKASRLDRGVPSRFSGSGSGTEFSLTISSLQPDDFATYYCLEYRSYIRTFGQGTKVEIK

SEQ ID NO: 123 - protein, 122 aa, Homo sapiens
EVQLVESGGGLVQPGRSLKLSCAASGFTFDDYAMHWVRQVPGKGLEWVSGISWRGDIIGYVKSVKGRFTISRDNAKNSLYLQLNSLRIEDTALYYCVKSYGSGSFYNAFDSWGQGTLVTVSS

SEQ ID NO: 124 - protein, 107 aa, Homo sapiens
DIQMTQSPSTLSASAGDRVTITCRASKSISSWLAWYQQKPGKAPKFLIYKAERLERGVPSRFSGSGSGTEFSLTISSLQPDDFATYYCLEYSSYIRTFGQGTKVEIK

SEQ ID NO: 125 - protein, 122 aa, Homo sapiens
EVQLVESGGGLVQPGRSLKLSCAASKFRFDDYAMHWVRQVPGKGLEWVSGISWRGDIIGYVDSVKGRFTISRDNEKNSLYLQLNSLRAEDTALYYCVKSYGSGSFYNAFDSWGQGTLVTVSS

SEQ ID NO: 126 - protein, 107 aa, Homo sapiens
DIQMTQSPSTLSASAGDRVTITCRASKSISSWLAWYQQKPGKAPKFLIYKASRLERGVPSRFSGSGSGTEFSLTISSLQPDDFATYYCLEYSSYIRTFGQGTKVEIK

SEQ ID NO: 127 - protein, 122 aa, Homo sapiens; identical to SEQ ID NO: 125

SEQ ID NO: 128 - protein, 107 aa, Homo sapiens
DIQMTQSPSTLSASAGDRVTITCRASQSISSWLAWYQQKPGKAPKFLIYKASRLERDVPSRFSGSGSGTEFSLTISSLQPDDFATYYCLEYRSYIRTFGQGTKVEIK

SEQ ID NO: 129 - protein, 122 aa, Homo sapiens
EVQLVESGGGLVQPGRSLKLSCAASGFTFDDYAMHWVRQVPGKGLEWVSGISWRGDIIGYVKSVKGRFTISRDNAKNSLYLQLNSLRAEDTALYYCVKSYGSGSFYNAFDSWGQGTLVTVSS

SEQ ID NO: 130 - protein, 107 aa, Homo sapiens
DIQMTQSPSTLSASVGDRVTITCRASKSIKSWLAWYQQKPGKAPKFLIYKASRLERGVPSRFSGSGSGTEFSLTISSLQPDDFATYYCLEYSSYIRTFGQGTKVEIK

SEQ ID NO: 131 - protein, 122 aa, Homo sapiens
EVQLVESGGGLVQPGRSLKLSCAASGFTFHDYAMHWVRQVPGKGLEWVSGISWRGDIKGYVDSVKGRFTISRDNAKNSLYLQLNSLRAEDTALYYCVKSYGSGSFYNAFDSWGQGTLVTVSS

SEQ ID NO: 132 - protein, 107 aa, Homo sapiens
DIQMTQSPSTLSASAGDRVTITCRASQSISSWLAWYQQKPGKAPKFLIYKASRLDRGVPSRFSGSGSGTEFSLTISSLQPDDFATYYCLEYSSYIRTFGQGTKVEIK

SEQ ID NO: 133 - protein, 122 aa, Homo sapiens
EVQLVESGGGLVQPGRSLKLSCAASGFTFHDYAMHWVRQVPGKGLEWVSGISWRGDITGYVDSVKGRFTISRDNAKNSLYLQLNSLRAEDTALYYCVKSYGSGSFYNAFDSWGQGTLVTVSS

SEQ ID NO: 134 - protein, 107 aa, Homo sapiens; identical to SEQ ID NO: 132

SEQ ID NO: 135 - protein, 122 aa, Homo sapiens
EVQLVESGGGLVQPGRSLKLSCAASGFTFHDYAMHWVRQVPGKGLEWVSGISWRGDIIGYVDSVKGRFTISRDNKKNSLYLQLNSLRAEDTALYYCVKSYGSGSFYNAFDSWGQGTLVTVSS

SEQ ID NO: 136 - protein, 107 aa, Homo sapiens; identical to SEQ ID NO: 132

SEQ ID NO: 137 - protein, 122 aa, Homo sapiens
EVQLVESGGGLVQPGRSLKLSCAASKFTFDDYAMHWVRQVPGKGLEWVSGISWRGDIIGYVDSVKGRFTISRDNAKNSLYLQLNSLRAEDTALYYCVKSYGSGSFYNAFDSWGQGTLVTVSS

SEQ ID NO: 138 - protein, 107 aa, Homo sapiens; identical to SEQ ID NO: 4

SEQ ID NO: 139 - protein, 122 aa, Homo sapiens
EVQLVESGGGLVQPGRSLKLSCAASGFTFDDYAMHWVRQVPGKGLEWVSGISWRGDIKGYVDSVKGRFTISRDNAKNSLYLQLNSLRAEDTALYYCVKSYGSGSFYNAFDSWGQGTLVTVSS

SEQ ID NO: 140 - protein, 107 aa, Homo sapiens; identical to SEQ ID NO: 4

SEQ ID NO: 141 - protein, 122 aa, Homo sapiens
EVQLVESGGGLVQPGRSLKLSCAASGFTFDDYAMHWVRQVPGKGLEWVSGISWRGDIGGYVDSVKGRFTISRDNAKNSLYLQLNSLRAEDTALYYCVKSYGSGSFYNAFDSWGQGTLVTVSS

SEQ ID NO: 142 - protein, 107 aa, Homo sapiens; identical to SEQ ID NO: 4

SEQ ID NO: 143 - protein, 122 aa, Homo sapiens
EVQLVESGGGLVQPGRSLKLSCAASGFTFHDYAMHWVRQVPGKGLEWVSGISWRGDIIGYVDSVKGRFTISRDNAKNSLYLQLNSLRAEDTALYYCVKSYGSGSFYNAFDSWGQGTLVTVSS

SEQ ID NO: 144 - protein, 107 aa, Homo sapiens
DIQMTQSPSTLSASAGDRVTISCRASQSISSWLAWYQQKPGKAPKFLIYKASRLDRGVPSRFSGSGSGTEFTLTISSLQPDDFATYYCLEYNSYIRTFGQGTKVEIK

SEQ ID NO: 145 - protein, 122 aa, Homo sapiens
EVQLVESGGGLVQPGRSLKLSCAASGFTFDDYAMHWVRQVPGKGLEWVSGISWKGDIIGYVDSVKGRFTISRDNAKNSLYLQLNSLRAEDTALYYCVKSYGSGSFYNAFDSWGQGTLVTVSS

SEQ ID NO: 146 - protein, 107 aa, Homo sapiens
DIQMTQSPSTLSASAGDRVTITCRASQSISEWLAWYQQKPGKAPKFLIYKASRLDRGVPSRFSGSGSGTEFSLTISSLQPDDFATYYCLEYSSYIRTFGQGTKVEIK

SEQ ID NO: 147 - protein, 122 aa, Homo sapiens; identical to SEQ ID NO: 123

SEQ ID NO: 148 - protein, 107 aa, Homo sapiens; identical to SEQ ID NO: 126

SEQ ID NO: 149 - protein, 122 aa, Homo sapiens; identical to SEQ ID NO: 3

SEQ ID NO: 150 - protein, 107 aa, Homo sapiens; identical to SEQ ID NO: 4

SEQ ID NO: 151 - protein, 120 aa, Homo sapiens; identical to SEQ ID NO: 21

SEQ ID NO: 152 - protein, 109 aa, Homo sapiens; identical to SEQ ID NO: 22

SEQ ID NO: 153 - protein, 122 aa, Homo sapiens; identical to SEQ ID NO: 3

SEQ ID NO: 154 - protein, 107 aa, Homo sapiens; identical to SEQ ID NO: 146

SEQ ID NO: 155 - protein, 120 aa, Homo sapiens; identical to SEQ ID NO: 21

SEQ ID NO: 156 - protein, 109 aa, Homo sapiens; identical to SEQ ID NO: 22

SEQ ID NO: 157 - protein, 120 aa, Homo sapiens; identical to SEQ ID NO: 21

SEQ ID NO: 158 - protein, 109 aa, Homo sapiens; identical to SEQ ID NO: 22

SEQ ID NO: 159 - protein, 119 aa, Homo sapiens; identical to SEQ ID NO: 91

SEQ ID NO: 160 - protein, 112 aa, Homo sapiens; identical to SEQ ID NO: 102

SEQ ID NO: 161 - protein, 123 aa, Homo sapiens
EVQLVESGGGLVKPGGSLRLSCAASGFTFSNAHMNWVRQAPGKGLEWVGRIKRKTDGGTTDYAAPVKGRFTISRDDSKNTLYLQMNSLKTEDTAVYYCTTDGIAVAGPLRVYWGQGTLVTVSS

SEQ ID NO: 162 - protein, 113 aa, Homo sapiens; identical to SEQ ID NO: 94

SEQ ID NO: 163 - protein, 119 aa, Homo sapiens; identical to SEQ ID NO: 91

SEQ ID NO: 164 - protein, 112 aa, Homo sapiens; identical to SEQ ID NO: 102

SEQ ID NO: 165 - protein, 123 aa, Homo sapiens
EVQLVESGGGLVKPGGSLRLSCAASGFTFSDAWMNWVRQAPGKGLEWVGRIKRKTDGGTTDYAAPVKGRFTISRDDSKNTLYLQMDSLKTEDTAVYYCTTDGIAVAGPLRVYWGQGTLVTVSS

SEQ ID NO: 166 - protein, 113 aa, Homo sapiens; identical to SEQ ID NO: 94

SEQ ID NO: 167 - protein, 119 aa, Homo sapiens; identical to SEQ ID NO: 91

SEQ ID NO: 168 - protein, 112 aa, Homo sapiens; identical to SEQ ID NO: 102

SEQ ID NO: 169 - protein, 123 aa, Homo sapiens
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDGIAVAGPLRVYWGQGTLVTVSS

SEQ ID NO: 170 - protein, 113 aa, Homo sapiens
DIVMTQSPDSLAVSLGERATINCKSSQSVLYSSNNGNYLVWYQQKPGQPPKLLIYWASTRESGVPDRFSGSGSGTDFTLTISSLQAEDVAVYYCQQYYSIPRTFGQGTKVEIK

SEQ ID NO: 171 - protein, 122 aa, Homo sapiens; identical to SEQ ID NO: 143

SEQ ID NO: 172 - protein, 107 aa, Homo sapiens; identical to SEQ ID NO: 132

SEQ ID NO: 173 - protein, 123 aa, Homo sapiens; identical to SEQ ID NO: 169

SEQ ID NO: 174 - protein, 113 aa, Homo sapiens; identical to SEQ ID NO: 170

SEQ ID NO: 175 - protein, 119 aa, Homo sapiens; identical to SEQ ID NO: 91

SEQ ID NO: 176 - protein, 112 aa, Homo sapiens; identical to SEQ ID NO: 102

SEQ ID NO: 177 - protein, 122 aa, Homo sapiens; identical to SEQ ID NO: 143

SEQ ID NO: 178 - protein, 107 aa, Homo sapiens
DIQMTQSPSTLSASAGDRVTITCRASQSISSWLAWYQQKPGKAPKFLIYKASRLDRGTPSRFSGSGSGTEFSLTISSLQPDDFATYYCLEYSSYIRTFGQGTKVEIK

SEQ ID NO: 179 - protein, 120 aa, Homo sapiens; identical to SEQ ID NO: 21

SEQ ID NO: 180 - protein, 109 aa, Homo sapiens; identical to SEQ ID NO: 22

SEQ ID NO: 181 - protein, 122 aa, Homo sapiens; identical to SEQ ID NO: 143

SEQ ID NO: 182 - protein, 107 aa, Homo sapiens; identical to SEQ ID NO: 178

SEQ ID NO: 183 - protein, 120 aa, Homo sapiens; identical to SEQ ID NO: 21

SEQ ID NO: 184 - protein, 109 aa, Homo sapiens
EIVLTQSPGTLSLSPGERATLSCRASQSVSSSYLAWYQQKPGQAPRLLIYGASSRTRGIPDRFSGSGSGTDFTLTISRLEPEDFAVYYCQQFGSSRLFTFGEGTKLEIK

SEQ ID NO: 185 - protein, 123 aa, Homo sapiens; identical to SEQ ID NO: 165

SEQ ID NO: 186 - protein, 113 aa, Homo sapiens; identical to SEQ ID NO: 94

SEQ ID NO: 187 - protein, 119 aa, Homo sapiens
EVQLVQSGAEVKKPGESLRISCKGSGYSFRPYWIGWVRQMPGKGLEWMGMIDPSDSYTDYGPSFEGHVTISADKSISTAYLQWSSLKASDTAMYYCARVRIRSGAFDIWGQGTMVTVSS

SEQ ID NO: 188 - protein, 112 aa, Homo sapiens
DIVMTQSPLSLPVTPGEPASISCRSSQRLLHRTGYNYLNWYLQKPGQSPQLLIYLGSRRASGVPDRFSGSGSGTDFTLKISRVEAEDVGVYYCMQVLHIPYTFGQGTKLEIK

SEQ ID NO: 189 - protein, 5 aa, synthetic construct
  REGION 1..5: note = Synthetic
DYAMH

SEQ ID NO: 190 - protein, 17 aa, synthetic construct
  REGION 1..17: note = Synthetic
GISWRGDIIGYVDSVKG

SEQ ID NO: 191 - protein, 13 aa, synthetic construct
  REGION 1..13: note = Synthetic
SYGSGSFYNAFDS

SEQ ID NO: 192 - protein, 11 aa, synthetic construct
  REGION 1..11: note = Synthetic
RASQSISSWLA

SEQ ID NO: 193 - protein, 7 aa, synthetic construct
  REGION 1..7: note = Synthetic
KASRLDR

SEQ ID NO: 194 - protein, 9 aa, synthetic construct
  REGION 1..9: note = Synthetic
LEYSSYIRT

SEQ ID NO: 195 - protein, 5 aa, synthetic construct
  REGION 1..5: note = Synthetic
TSWIV

SEQ ID NO: 196 - protein, 17 aa, synthetic construct
  REGION 1..17: note = Synthetic
MIDPSDSFTSYSPSFQG

SEQ ID NO: 197 - protein, 11 aa, synthetic construct
  REGION 1..11: note = Synthetic
LHYYHSEEFDV

SEQ ID NO: 198 - protein, 12 aa, synthetic construct
  REGION 1..12: note = Synthetic
RASQSVSSSYLA

SEQ ID NO: 199 - protein, 7 aa, synthetic construct
  REGION 1..7: note = Synthetic
GASSRAR

SEQ ID NO: 200 - protein, 10 aa, synthetic construct
  REGION 1..10: note = Synthetic
QQFGSSRLFT

SEQ ID NO: 201 - protein, 11 aa, synthetic construct
  REGION 1..11: note = Synthetic
RASQSISSWLA

SEQ ID NO: 202 - protein, 7 aa, synthetic construct
  REGION 1..7: note = Synthetic
GASSRTR